SEQUENCE LISTING (51 sequences)

SEQ ID NO: 1 - DNA, 3467 bp, Artificial Sequence
  PLASMID SEQUENCE BASED ON A VARIETY OF SOURCES
atgaccatgattacgccaagcgcgcaattaaccctcactaaagggaacaaaagctgggtaccgggccccccctcgaggtcgacggtatcgataagcttgatatccactgtggaattcgcccttaagggcgaattccacattgggctgcagcccgggggatccactagttctagagcggccgcaccgcgggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgattaaattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccttaatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttacagttaattaaagggaacaaaagctggcatgtaccgttcgtatagcatacattatacgaacggtacgctccaattcgccctttaattaactgttccaactttcaccataatgaaataagatcactaccgggcgtattttttgagttgtcgagattttcaggagctaaggaagctaaaatggagaaaaaaatcactggatataccaccgagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagct

SEQ ID NO: 2 - DNA, 7433 bp, Artificial Sequence
  PLASMID SEQUENCE BASED ON A VARIETY OF SOURCES
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttaaggtgcacggcccacgtggccactagtacttctcgagctctgtacatgtccgcggtcgcgacgtacgcgtatcgatggcgccagctgcagagcgttccagtggatatttgctgggggttaatgaaacattgtggcggaacccagggacaatgtgaccaaaaaattcagggatatcaataagtattaggtatatggatcataattgtatgcccgactattgcttaaactgactgaccactgaccttaagagtaatggcgtgcaaggcccagtgatcaatttcattatttttcattatttcatctccattgtccctgaaaatcagttgtgtcgcccctctacacagcccagaactatggtaaaggcgcacgaaaaaccgccaggtaaactcttctcaacccccaaaacgccctctgtttacccatggaaaaaacgacaattacaagaaagtaaaacttatgtcatctataagcttcgtgtatattaacttcctgttacaaagctttacaaaactctcattaatcctttagactaagtttagtcagttccaatctgaacatcgacaaatacataaggaattataaccatatgcatcctaggcctattaatattccggagtatacgtagccggctaacgttatcggcattttcttttgcgtttttatttgttaactgttaattgtccttgttcaaggatgctgtctttgacaacagatgttttcttgcctttgatgttcagcaggaagcttggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacctccaggccttttgcagaagagatatttttaattgtggacgaatcgaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcgatcctctagctagagtcgacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcgacggatcctaattccttggtgtaatgccaactgaataatctgcaaattgcactctccttcaatggggggtgctttttgcttgactgagtaatcttctgattgctgatcttgattgccatcgatcgccggggagtccggggcagttaccattagagagtctagagaattaatccatcttcgatagaggaattatgggggaagaacctgtgccggcggataaagcattaggcaagaaattcaagaaaaaaaatgcctcctggagcattgaagaaagcgaagctctgtaccgggttgaggcctggggggcaccttattttgccattaatgccgctggtaacataaccgtctctcccaacggcgatcggggcggttcgttagatttgttggaactggtggaagccctgcggcaaagaaagctcggcttacccctattaattcgtttttccgatattttggccgatcgcctagagcgattgaatagttgttttgccaaggcgatcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 3 - DNA, 7489 bp, Artificial Sequence
  PLASMID SEQUENCE BASED ON A VARIETY OF SOURCES
atgaccatgattacgccaagcgcgcaattaaccctcactaaagggaacaaaagctgggtaccgggccccccctcgaggtcgacggtatcgataagcttgatatccactgtggaattcgcccttgccaattgcagacgactacgggcaaagaggcgacgggtattcatggcgatagggtgaaccgatagccttgaccgggaactgttttaattgggcaaggacaattttgttgagctagcttgcgtcgtatcaaacgcatttgggccgccaccacattactcatgggctcctcatcaagatcccacagttgttgccggatcttgctaccggaaatgatccgctctgggttttgcatcagatattgaaaaatttgaaattctcttacggttaaagcaatttcctgtctttctaggtttagtggctccgagatagttaccgataacagattattactgggatcaaggctgaagttgcccaaagttaaaatttgcggttggaattgtggcgatcgccgttgtagtgcccgcagtcttgctaatagctctgccatcacaaacggttttgttagatagtcatctgccccggcatctagtccttcgacacggttttccggttctcctaacgctgttaacatcaacaccggcaaggaattaccctgggttctcagtttttgacagagttccaaacccgataatcccggcagtaaccaatccacaatggcaagggtgtattccgtccattgattttccaaataatcccaagcttgggagccatccgtcacccaatccaccacatacttttcactaactagcactttcttaatagccattcccaaatccgtctcatcttccaccagcaaaattcgcatcgcctctgccttttttataacggtctgatcttagcgggggaaggagattttcacctgaatttcataccccctttggcagactgggaaaatcttggacaaattcccaatttgaggtggtgtgatgccagaaaaacatgatctgttaaccgccatgatggcggcaaaggaacagggcatcggggcaatccttgcgtttgcaatggcgtaccttcgcggtcggtataatggcggtgcgtttaagaaaacactaatagacgcaacgatgtgcgccattatcgcctggttcattcgtgaccttttagtcttcgccggactgagtagcaatcttgcttacatagcgagtgtgtttatcggctacatcggcacagactcgattggttcgctaatcaaacgcttcgctgctaaaaaagccggagtcgatgatgcaaatcagcagtaacggaatcaccagattaaaacgtgaagaaggtgagagactaaaagcctattcagatagcagggggataccaaccattggggttgggcataccggaaaagtggatggtaattctgtcgcatcagggatgacaatcaccgccgaaaaatcttctgaactgcttaaagaggatttgcagtgggttgaagatgcgataagtagtcttgttcgcgtcccgctaaatcagaaccagtatgatgcgctatgtagcctgatattcaacataggtaaatcagcatttgccggctctaccgttcttcgccagttgaatttaaagaattaccaggcagcagcagatgctttcctgttatggaaaaaagctggtaaagaccctgatattctccttccacggaggcggcgagaaagagcgctgttcttatcgtgagtcgtattaaggcaattattgcgtctgtcattatctgcatcatcgtctgtctttcgtgggctgttaatcattatcgtgataacgccatcacctacaaagagcagcgcgataaagccacatcaatcatcgctgatatgcagaagcgtcaacgagatgtagcagaactcgatgccagatacacaaaggagcttgctgatgctaacgcgactatcgaaactctccgcgctgatgtttctgctgggcgtaagcgcctgcaagtctccgccacctgtccaaagtcaacgaccggagccagcggcatgggcgatggagaaagcccaagacttacagcagatgctgaactcaattattaccgtctccgaagtggaatcgacaggataaccgcgcaggttaactacctgcaggagtacatcaggagtcagtgcttaaaataatctagaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcgacggatccggggaattcgtaatcatggtcatagctgtttcctgtgtgaaattgttatccgctcacaattccacacaacatacgagccggaagcataggatcctgccccagggggtttcttgattggcggtggccaagagctattagtacggggtctgggtcaaatgcagtccattgaagacctacggcgatcggtggtgaaagtggtggacggcaaaccaattctgttggaggacgttgctgaagttaaaaccggcagtgccctgaagcggggggatgggagctttaacggtcaaccggcgatcgtcatgatggtcaataaacagcccgatgtggatacgcccacagtgactaaagcagtagaagcagtggttgaatctctaaaacccacgtttcctgccgatgtacaaattgcccaaacctttcgtcaagctaactttattgattccgccattcgcaacgtcagcacttccctcttagaagggattgtcatcgtttcggtgattatgctgatttttttaatgaactggcgcacggcggcgattaccctaacagcgattcccctctccctgctaattggtttgatgttcatgaaagcctggggattgggcattaataccatgaccctaggggggctagtggtggcgatcggctccgtggtagatgactccattgtggatatggaaaattgctatcggggactacgcactaaccaggccgagggcaatcccaaacatcctttgcgggtagtttatgaaacctcggtggaagtccgattagcagtgattttttccacggtgatcatcgtggtggttttcgcgcccattttcagcttaacgggggtagaagggcgtatttttgcccccatgggtttagcctatctactctgtatcggtgcttccaccctagtggccatgaccgtttccccggctttgtgtgggattctcttggctaaccaacgactgccaagggcgaattccacattgggctgcagcccgggggatccactagttctagagcggccgcaccgcgggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgattaaattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccttaatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttacagttaattaaagggaacaaaagctggcatgtaccgttcgtatagcatacattatacgaacggtacgctccaattcgccctttaattaactgttccaactttcaccataatgaaataagatcactaccgggcgtattttttgagttgtcgagattttcaggagctaaggaagctaaaatggagaaaaaaatcactggatataccaccgagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagct

SEQ ID NO: 4 - DNA, 8924 bp, Artificial Sequence
  PLASMID SEQUENCE BASED ON A VARIETY OF SOURCES
atgaccatgattacgccaagcgcgcaattaaccctcactaaagggaacaaaagctgggtaccgggccccccctcgaggtcgacggtatcgataagcttgatatccactgtggaattcgcccttggtaccgccaattgcagacgactacgggcaaagaggcgacgggtattcatggcgatagggtgaaccgatagccttgaccgggaactgttttaattgggcaaggacaattttgttgagctagcttgcgtcgtatcaaacgcatttgggccgccaccacattactcatgggctcctcatcaagatcccacagttgttgccggatcttgctaccggaaatgatccgctctgggttttgcatcagatattgaaaaatttgaaattctcttacggttaaagcaatttcctgtctttctaggtttagtggctccgagatagttaccgataacagattattactgggatcaaggctgaagttgcccaaagttaaaatttgcggttggaattgtggcgatcgccgttgtagtgcccgcagtcttgctaatagctctgccatcacaaacggttttgttagatagtcatctgccccggcatctagtccttcgacacggttttccggttctcctaacgctgttaacatcaacaccggcaaggaattaccctgggttctcagtttttgacagagttccaaacccgataatcccggcagtaaccaatccacaatggcaagggtgtattccgtccattgattttccaaataatcccaagcttgggagccatccgtcacccaatccaccacatacttttcactaactagcactttcttaatagccattcccaaatccgtctcatcttccaccagcaaaattcgcatcgcctctgccttttttataacggtctgatcttagcgggggaaggagattttcacctgaatttcataccccctttggcagactgggaaaatcttggacaaattcccaatttgaggtggtgtgatgccagaaaaacatgatctgttaaccgccatgatggcggcaaaggaacagggcatcggggcaatccttgcgtttgcaatggcgtaccttcgcggtcggtataatggcggtgcgtttaagaaaacactaatagacgcaacgatgtgcgccattatcgcctggttcattcgtgaccttttagtcttcgccggactgagtagcaatcttgcttacatagcgagtgtgtttatcggctacatcggcacagactcgattggttcgctaatcaaacgcttcgctgctaaaaaagccggagtcgatgatgcaaatcagcagtaacatatgatgcatcctaggcctattaatattccggagtatacgtagccggctaacgttatcggcattttcttttgcgtttttatttgttaactgttaattgtccttgttcaaggatgctgtctttgacaacagatgttttcttgcctttgatgttcagcaggaagcttggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacctccaggccttttgcagaagagatatttttaattgtggacgaatcgaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcgatcctctagctagagtcgacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcgacggatctctagacacattgctccttttgtgcgtaacgatagggtcagcactcaaaaatcgcatttttaaacgtgaattattatctcttctggctgtattaacaacggtgggagagatttttttaccacattttttctggttaatttccatgaccattacccaaaacacaactcgccactatcatcatcgtcgaaggtctcaacaatcatcttggtcacgccatttttttctggccactttattatttactctttgcttggcagcttttattagaaagtctcctgaaactgaaaacatcaattccttttttggccatttaccatccctagccatggaaggaggagatccttacattagagctttaatgcggacaatttcagccagtgaatctaatgctaaaaatccctacgttttactctatggcggtcaacatacccatgatttaagtcgccatcccaatgcttgtattgccatcaaaacagatgttaaccaagggcattgctccacagcggcaggacgttatcaatttttgactaagacttggcaggaaaaagcggctttgtatcaccctcaacgtcatttgggaaaatcacactataactttgagcctgaatttcaggatttagtaacctatcgatggttgaccgataaacaccactggggcatggacttttccacccaattacaacagggaaatatcgaacaagtgttgaaaaaactttctggcacttggacaagtttgggttacggcattgaagacaatagaatgaccgcttctttacccaaaatttatcaaaaactattagcagaagaacttgaccaagctaattaatattcgattcagtaccaagtactattgcggggacaggacgtttctcaaggccctcatcaatatcccccctgggggcatagaatagagatcaattttctaccccaaacccccacaatgggcaaactaccgcctatctttcactgcttgcggaaccgtctatttgctcagctatacctagcccaagccattagttccgcggaagggcgaattccacattgggctgcagcccgggggatccactagttctagagcggccgcaccgcgggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgattaaattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccttaatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttacagttaattaaagggaacaaaagctggcatgtaccgttcgtatagcatacattatacgaacggtacgctccaattcgccctttaattaactgttccaactttcaccataatgaaataagatcactaccgggcgtattttttgagttgtcgagattttcaggagctaaggaagctaaaatggagaaaaaaatcactggatataccaccgagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagct

SEQ ID NO: 5 - DNA, 5688 bp, Artificial Sequence
  PLASMID SEQUENCE BASED ON A VARIETY OF SOURCES
atgaccatgattacgccaagcgcgcaattaaccctcactaaagggaacaaaagctgggtaccgggccccccctcgaggtcgacggtatcgataagcttgatatccactgtggaattcgcccttggtaccgccaattgcagacgactacgggcaaagaggcgacgggtattcatggcgatagggtgaaccgatagccttgaccgggaactgttttaattgggcaaggacaattttgttgagctagcttgcgtcgtatcaaacgcatttgggccgccaccacattactcatgggctcctcatcaagatcccacagttgttgccggatcttgctaccggaaatgatccgctctgggttttgcatcagatattgaaaaatttgaaattctcttacggttaaagcaatttcctgtctttctaggtttagtggctccgagatagttaccgataacagattattactgggatcaaggctgaagttgcccaaagttaaaatttgcggttggaattgtggcgatcgccgttgtagtgcccgcagtcttgctaatagctctgccatcacaaacggttttgttagatagtcatctgccccggcatctagtccttcgacacggttttccggttctcctaacgctgttaacatcaacaccggcaaggaattaccctgggttctcagtttttgacagagttccaaacccgataatcccggcagtaaccaatccacaatggcaagggtgtattccgtccattgattttccaaataatcccaagcttgggagccatccgtcacccaatccaccacatacttttcactaactagcactttcttaatagccattcccaaatccgtctcatcttccaccagcaaaattcgcatcgcctctgccttttttataacggtctgatcttagcgggggaaggagattttcacctgaatttcataccccctttggcagactgggaaaatcttggacaaattcccaatttgaggtggtgtgatgccagaaaaacatgatctgttaaccgccatgatggcggcaaaggaacagggcatcggggcaatccttgcgtttgcaatggcgtaccttcgcggtcggtataatggcggtgcgtttaagaaaacactaatagacgcaacgatgtgcgccattatcgcctggttcattcgtgaccttttagtcttcgccggactgagtagcaatcttgcttacatagcgagtgtgtttatcggctacatcggcacagactcgattggttcgctaatcaaacgcttcgctgctaaaaaagccggagtcgatgatgcaaatcagcagtaacatatgtctagacacattgctccttttgtgcgtaacgatagggtcagcactcaaaaatcgcatttttaaacgtgaattattatctcttctggctgtattaacaacggtgggagagatttttttaccacattttttctggttaatttccatgaccattacccaaaacacaactcgccactatcatcatcgtcgaaggtctcaacaatcatcttggtcacgccatttttttctggccactttattatttactctttgcttggcagcttttattagaaagtctcctgaaactgaaaacatcaattccttttttggccatttaccatccctagccatggaaggaggagatccttacattagagctttaatgcggacaatttcagccagtgaatctaatgctaaaaatccctacgttttactctatggcggtcaacatacccatgatttaagtcgccatcccaatgcttgtattgccatcaaaacagatgttaaccaagggcattgctccacagcggcaggacgttatcaatttttgactaagacttggcaggaaaaagcggctttgtatcaccctcaacgtcatttgggaaaatcacactataactttgagcctgaatttcaggatttagtaacctatcgatggttgaccgataaacaccactggggcatggacttttccacccaattacaacagggaaatatcgaacaagtgttgaaaaaactttctggcacttggacaagtttgggttacggcattgaagacaatagaatgaccgcttctttacccaaaatttatcaaaaactattagcagaagaacttgaccaagctaattaatattcgattcagtaccaagtactattgcggggacaggacgtttctcaaggccctcatcaatatcccccctgggggcatagaatagagatcaattttctaccccaaacccccacaatgggcaaactaccgcctatctttcactgcttgcggaaccgtctatttgctcagctatacctagcccaagccattagttccgcggaagggcgaattccacattgggctgcagcccgggggatccactagttctagagcggccgcaccgcgggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgattaaattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccttaatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttacagttaattaaagggaacaaaagctggcatgtaccgttcgtatagcatacattatacgaacggtacgctccaattcgccctttaattaactgttccaactttcaccataatgaaataagatcactaccgggcgtattttttgagttgtcgagattttcaggagctaaggaagctaaaatggagaaaaaaatcactggatataccaccgagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagct

SEQ ID NO: 6 - DNA, 6543 bp, Artificial Sequence
  PLASMID SEQUENCE BASED ON A VARIETY OF SOURCES
atgaccatgattacgccaagcgcgcaattaaccctcactaaagggaacaaaagctgggtaccgggccccccctcgaggtcgacggtatcgataagcttgatatccactgtggaattcgcccttggtaccgccaattgcagacgactacgggcaaagaggcgacgggtattcatggcgatagggtgaaccgatagccttgaccgggaactgttttaattgggcaaggacaattttgttgagctagcttgcgtcgtatcaaacgcatttgggccgccaccacattactcatgggctcctcatcaagatcccacagttgttgccggatcttgctaccggaaatgatccgctctgggttttgcatcagatattgaaaaatttgaaattctcttacggttaaagcaatttcctgtctttctaggtttagtggctccgagatagttaccgataacagattattactgggatcaaggctgaagttgcccaaagttaaaatttgcggttggaattgtggcgatcgccgttgtagtgcccgcagtcttgctaatagctctgccatcacaaacggttttgttagatagtcatctgccccggcatctagtccttcgacacggttttccggttctcctaacgctgttaacatcaacaccggcaaggaattaccctgggttctcagtttttgacagagttccaaacccgataatcccggcagtaaccaatccacaatggcaagggtgtattccgtccattgattttccaaataatcccaagcttgggagccatccgtcacccaatccaccacatacttttcactaactagcactttcttaatagccattcccaaatccgtctcatcttccaccagcaaaattcgcatcgcctctgccttttttataacggtctgatcttagcgggggaaggagattttcacctgaatttcataccccctttggcagactgggaaaatcttggacaaattcccaatttgaggtggtgtgatgccagaaaaacatgatctgttaaccgccatgatggcggcaaaggaacagggcatcggggcaatccttgcgtttgcaatggcgtaccttcgcggtcggtataatggcggtgcgtttaagaaaacactaatagacgcaacgatgtgcgccattatcgcctggttcattcgtgaccttttagtcttcgccggactgagtagcaatcttgcttacatagcgagtgtgtttatcggctacatcggcacagactcgattggttcgctaatcaaacgcttcgctgctaaaaaagccggagtcgatgatgcaaatcagcagtaacggaatcaccagattaaaacgtgaagaaggtgagagactaaaagcctattcagatagcagggggataccaaccattggggttgggcataccggaaaagtggatggtaattctgtcgcatcagggatgacaatcaccgccgaaaaatcttctgaactgcttaaagaggatttgcagtgggttgaagatgcgataagtagtcttgttcgcgtcccgctaaatcagaaccagtatgatgcgctatgtagcctgatattcaacataggtaaatcagcatttgccggctctaccgttcttcgccagttgaatttaaagaattaccaggcagcagcagatgctttcctgttatggaaaaaagctggtaaagaccctgatattctccttccacggaggcggcgagaaagagcgctgttcttatcgtgagtcgtattaaggcaattattgcgtctgtcattatctgcatcatcgtctgtctttcgtgggctgttaatcattatcgtgataacgccatcacctacaaagagcagcgcgataaagccacatcaatcatcgctgatatgcagaagcgtcaacgagatgtagcagaactcgatgccagatacacaaaggagcttgctgatgctaacgcgactatcgaaactctccgcgctgatgtttctgctgggcgtaagcgcctgcaagtctccgccacctgtccaaagtcaacgaccggagccagcggcatgggcgatggagaaagcccaagacttacagcagatgctgaactcaattattaccgtctccgaagtggaatcgacaggataaccgcgcaggttaactacctgcaggagtacatcaggagtcagtgcttaaaataacatatgtctagacacattgctccttttgtgcgtaacgatagggtcagcactcaaaaatcgcatttttaaacgtgaattattatctcttctggctgtattaacaacggtgggagagatttttttaccacattttttctggttaatttccatgaccattacccaaaacacaactcgccactatcatcatcgtcgaaggtctcaacaatcatcttggtcacgccatttttttctggccactttattatttactctttgcttggcagcttttattagaaagtctcctgaaactgaaaacatcaattccttttttggccatttaccatccctagccatggaaggaggagatccttacattagagctttaatgcggacaatttcagccagtgaatctaatgctaaaaatccctacgttttactctatggcggtcaacatacccatgatttaagtcgccatcccaatgcttgtattgccatcaaaacagatgttaaccaagggcattgctccacagcggcaggacgttatcaatttttgactaagacttggcaggaaaaagcggctttgtatcaccctcaacgtcatttgggaaaatcacactataactttgagcctgaatttcaggatttagtaacctatcgatggttgaccgataaacaccactggggcatggacttttccacccaattacaacagggaaatatcgaacaagtgttgaaaaaactttctggcacttggacaagtttgggttacggcattgaagacaatagaatgaccgcttctttacccaaaatttatcaaaaactattagcagaagaacttgaccaagctaattaatattcgattcagtaccaagtactattgcggggacaggacgtttctcaaggccctcatcaatatcccccctgggggcatagaatagagatcaattttctaccccaaacccccacaatgggcaaactaccgcctatctttcactgcttgcggaaccgtctatttgctcagctatacctagcccaagccattagttccgcggaagggcgaattccacattgggctgcagcccgggggatccactagttctagagcggccgcaccgcgggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgattaaattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccttaatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttacagttaattaaagggaacaaaagctggcatgtaccgttcgtatagcatacattatacgaacggtacgctccaattcgccctttaattaactgttccaactttcaccataatgaaataagatcactaccgggcgtattttttgagttgtcgagattttcaggagctaaggaagctaaaatggagaaaaaaatcactggatataccaccgagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagct

SEQ ID NO: 7 - DNA, 6606 bp, Artificial Sequence
  PLASMID SEQUENCE BASED ON A VARIETY OF SOURCES
atgaccatgattacgccaagcgcgcaattaaccctcactaaagggaacaaaagctgggtaccgggccccccctcgaggtcgacggtatcgataagcttgatatccactgtggaattcgcccttggtaccgccaattgcagacgactacgggcaaagaggcgacgggtattcatggcgatagggtgaaccgatagccttgaccgggaactgttttaattgggcaaggacaattttgttgagctagcttgcgtcgtatcaaacgcatttgggccgccaccacattactcatgggctcctcatcaagatcccacagttgttgccggatcttgctaccggaaatgatccgctctgggttttgcatcagatattgaaaaatttgaaattctcttacggttaaagcaatttcctgtctttctaggtttagtggctccgagatagttaccgataacagattattactgggatcaaggctgaagttgcccaaagttaaaatttgcggttggaattgtggcgatcgccgttgtagtgcccgcagtcttgctaatagctctgccatcacaaacggttttgttagatagtcatctgccccggcatctagtccttcgacacggttttccggttctcctaacgctgttaacatcaacaccggcaaggaattaccctgggttctcagtttttgacagagttccaaacccgataatcccggcagtaaccaatccacaatggcaagggtgtattccgtccattgattttccaaataatcccaagcttgggagccatccgtcacccaatccaccacatacttttcactaactagcactttcttaatagccattcccaaatccgtctcatcttccaccagcaaaattcgcatcgcctctgccttttttataacggtctgatcttagcgggggaaggagattttcacctgaatttcataccccctttggcagactgggaaaatcttggacaaattcccaatttgaggtggtgtgatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagcatatgtctagacacattgctccttttgtgcgtaacgatagggtcagcactcaaaaatcgcatttttaaacgtgaattattatctcttctggctgtattaacaacggtgggagagatttttttaccacattttttctggttaatttccatgaccattacccaaaacacaactcgccactatcatcatcgtcgaaggtctcaacaatcatcttggtcacgccatttttttctggccactttattatttactctttgcttggcagcttttattagaaagtctcctgaaactgaaaacatcaattccttttttggccatttaccatccctagccatggaaggaggagatccttacattagagctttaatgcggacaatttcagccagtgaatctaatgctaaaaatccctacgttttactctatggcggtcaacatacccatgatttaagtcgccatcccaatgcttgtattgccatcaaaacagatgttaaccaagggcattgctccacagcggcaggacgttatcaatttttgactaagacttggcaggaaaaagcggctttgtatcaccctcaacgtcatttgggaaaatcacactataactttgagcctgaatttcaggatttagtaacctatcgatggttgaccgataaacaccactggggcatggacttttccacccaattacaacagggaaatatcgaacaagtgttgaaaaaactttctggcacttggacaagtttgggttacggcattgaagacaatagaatgaccgcttctttacccaaaatttatcaaaaactattagcagaagaacttgaccaagctaattaatattcgattcagtaccaagtactattgcggggacaggacgtttctcaaggccctcatcaatatcccccctgggggcatagaatagagatcaattttctaccccaaacccccacaatgggcaaactaccgcctatctttcactgcttgcggaaccgtctatttgctcagctatacctagcccaagccattagttccgcggaagggcgaattccacattgggctgcagcccgggggatccactagttctagagcggccgcaccgcgggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgattaaattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccttaatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttacagttaattaaagggaacaaaagctggcatgtaccgttcgtatagcatacattatacgaacggtacgctccaattcgccctttaattaactgttccaactttcaccataatgaaataagatcactaccgggcgtattttttgagttgtcgagattttcaggagctaaggaagctaaaatggagaaaaaaatcactggatataccaccgagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagct

SEQ ID NO: 8 - DNA, 6962 bp, Artificial Sequence
  PLASMID SEQUENCE BASED ON A VARIETY OF SOURCES
atgaccatgattacgccaagcgcgcaattaaccctcactaaagggaacaaaagctgggtaccgggccccccctcgaggtcgacggtatcgataagcttgatatccactgtggaattcgcccttggtaccgccaattgcagacgactacgggcaaagaggcgacgggtattcatggcgatagggtgaaccgatagccttgaccgggaactgttttaattgggcaaggacaattttgttgagctagcttgcgtcgtatcaaacgcatttgggccgccaccacattactcatgggctcctcatcaagatcccacagttgttgccggatcttgctaccggaaatgatccgctctgggttttgcatcagatattgaaaaatttgaaattctcttacggttaaagcaatttcctgtctttctaggtttagtggctccgagatagttaccgataacagattattactgggatcaaggctgaagttgcccaaagttaaaatttgcggttggaattgtggcgatcgccgttgtagtgcccgcagtcttgctaatagctctgccatcacaaacggttttgttagatagtcatctgccccggcatctagtccttcgacacggttttccggttctcctaacgctgttaacatcaacaccggcaaggaattaccctgggttctcagtttttgacagagttccaaacccgataatcccggcagtaaccaatccacaatggcaagggtgtattccgtccattgattttccaaataatcccaagcttgggagccatccgtcacccaatccaccacatacttttcactaactagcactttcttaatagccattcccaaatccgtctcatcttccaccagcaaaattcgcatcgcctctgccttttttataacggtctgatcttagcgggggaaggagattttcacctgaatttcataccccctttggcagactgggaaaatcttggacaaattcccaatttgaggtggtgtgatgccagaaaaacatgatctgttaaccgccatgatggcggcaaaggaacagggcatcggggcaatccttgcgtttgcaatggcgtaccttcgcggtcggtataatggcggtgcgtttaagaaaacactaatagacgcaacgatgtgcgccattatcgcctggttcattcgtgaccttttagtcttcgccggactgagtagcaatcttgcttacatagcgagtgtgtttatcggctacatcggcacagactcgattggttcgctaatcaaacgcttcgctgctaaaaaagccggagtcgatgatgcaaatcagcagtaacatatgggatcctaattgtatgcccgactattgcttaaactgactgaccactgaccttaagagtaatggcgtgcaaggcccagtgatcaatttcattatttttcattatttcatctccattgtccctgaaaatcagttgtgtcgcccctctacacagcccagaactatggtaaaggcgcacgaaaaaccgccaggtaaactcttctcaacccccaaaacgccctctgtttacccatggaaaaaacgacaattacaagaaagtaaaacttatgtcatctataagcttcgtgtatattaacttcctgttacaaagctttacaaaactctcattaatcctttagactaagtttagtcagttccaatctgaacatcgacaaatacataaggaattataaccaaatgatgcaaatcagcagtaacggaatcaccagattaaaacgtgaagaaggtgagagactaaaagcctattcagatagcagggggataccaaccattggggttgggcataccggaaaagtggatggtaattctgtcgcatcagggatgacaatcaccgccgaaaaatcttctgaactgcttaaagaggatttgcagtgggttgaagatgcgataagtagtcttgttcgcgtcccgctaaatcagaaccagtatgatgcgctatgtagcctgatattcaacataggtaaatcagcatttgccggctctaccgttcttcgccagttgaatttaaagaattaccaggcagcagcagatgctttcctgttatggaaaaaagctggtaaagaccctgatattctccttccacggaggcggcgagaaagagcgctgttcttatcgtgagtcgtattaaggcaattattgcgtctgtcattatctgcatcatcgtctgtctttcgtgggctgttaatcattatcgtgataacgccatcacctacaaagagcagcgcgataaagccacatcaatcatcgctgatatgcagaagcgtcaacgagatgtagcagaactcgatgccagatacacaaaggagcttgctgatgctaacgcgactatcgaaactctccgcgctgatgtttctgctgggcgtaagcgcctgcaagtctccgccacctgtccaaagtcaacgaccggagccagcggcatgggcgatggagaaagcccaagacttacagcagatgctgaactcaattattaccgtctccgaagtggaatcgacaggataaccgcgcaggttaactacctgcaggagtacatcaggagtcagtgcttaaaataaggatcctctagacacattgctccttttgtgcgtaacgatagggtcagcactcaaaaatcgcatttttaaacgtgaattattatctcttctggctgtattaacaacggtgggagagatttttttaccacattttttctggttaatttccatgaccattacccaaaacacaactcgccactatcatcatcgtcgaaggtctcaacaatcatcttggtcacgccatttttttctggccactttattatttactctttgcttggcagcttttattagaaagtctcctgaaactgaaaacatcaattccttttttggccatttaccatccctagccatggaaggaggagatccttacattagagctttaatgcggacaatttcagccagtgaatctaatgctaaaaatccctacgttttactctatggcggtcaacatacccatgatttaagtcgccatcccaatgcttgtattgccatcaaaacagatgttaaccaagggcattgctccacagcggcaggacgttatcaatttttgactaagacttggcaggaaaaagcggctttgtatcaccctcaacgtcatttgggaaaatcacactataactttgagcctgaatttcaggatttagtaacctatcgatggttgaccgataaacaccactggggcatggacttttccacccaattacaacagggaaatatcgaacaagtgttgaaaaaactttctggcacttggacaagtttgggttacggcattgaagacaatagaatgaccgcttctttacccaaaatttatcaaaaactattagcagaagaacttgaccaagctaattaatattcgattcagtaccaagtactattgcggggacaggacgtttctcaaggccctcatcaatatcccccctgggggcatagaatagagatcaattttctaccccaaacccccacaatgggcaaactaccgcctatctttcactgcttgcggaaccgtctatttgctcagctatacctagcccaagccattagttccgcggaagggcgaattccacattgggctgcagcccgggggatccactagttctagagcggccgcaccgcgggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgattaaattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccttaatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttacagttaattaaagggaacaaaagctggcatgtaccgttcgtatagcatacattatacgaacggtacgctccaattcgccctttaattaactgttccaactttcaccataatgaaataagatcactaccgggcgtattttttgagttgtcgagattttcaggagctaaggaagctaaaatggagaaaaaaatcactggatataccaccgagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagct

SEQ ID NO: 9 - DNA, 6927 bp, Artificial Sequence
  PLASMID SEQUENCE BASED ON A VARIETY OF SOURCES
atgaccatgattacgccaagcgcgcaattaaccctcactaaagggaacaaaagctgggtaccgggccccccctcgaggtcgacggtatcgataagcttgatatccactgtggaattcgcccttggtaccgccaattgcagacgactacgggcaaagaggcgacgggtattcatggcgatagggtgaaccgatagccttgaccgggaactgttttaattgggcaaggacaattttgttgagctagcttgcgtcgtatcaaacgcatttgggccgccaccacattactcatgggctcctcatcaagatcccacagttgttgccggatcttgctaccggaaatgatccgctctgggttttgcatcagatattgaaaaatttgaaattctcttacggttaaagcaatttcctgtctttctaggtttagtggctccgagatagttaccgataacagattattactgggatcaaggctgaagttgcccaaagttaaaatttgcggttggaattgtggcgatcgccgttgtagtgcccgcagtcttgctaatagctctgccatcacaaacggttttgttagatagtcatctgccccggcatctagtccttcgacacggttttccggttctcctaacgctgttaacatcaacaccggcaaggaattaccctgggttctcagtttttgacagagttccaaacccgataatcccggcagtaaccaatccacaatggcaagggtgtattccgtccattgattttccaaataatcccaagcttgggagccatccgtcacccaatccaccacatacttttcactaactagcactttcttaatagccattcccaaatccgtctcatcttccaccagcaaaattcgcatcgcctctgccttttttataacggtctgatcttagcgggggaaggagattttcacctgaatttcataccccctttggcagactgggaaaatcttggacaaattcccaatttgaggtggtgtgatgccagaaaaacatgatctgttaaccgccatgatggcggcaaaggaacagggcatcggggcaatccttgcgtttgcaatggcgtaccttcgcggtcggtataatggcggtgcgtttaagaaaacactaatagacgcaacgatgtgcgccattatcgcctggttcattcgtgaccttttagtcttcgccggactgagtagcaatcttgcttacatagcgagtgtgtttatcggctacatcggcacagactcgattggttcgctaatcaaacgcttcgctgctaaaaaagccggagtcgatgatgcaaatcagcagtaacatatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagcatatgtctagacacattgctccttttgtgcgtaacgatagggtcagcactcaaaaatcgcatttttaaacgtgaattattatctcttctggctgtattaacaacggtgggagagatttttttaccacattttttctggttaatttccatgaccattacccaaaacacaactcgccactatcatcatcgtcgaaggtctcaacaatcatcttggtcacgccatttttttctggccactttattatttactctttgcttggcagcttttattagaaagtctcctgaaactgaaaacatcaattccttttttggccatttaccatccctagccatggaaggaggagatccttacattagagctttaatgcggacaatttcagccagtgaatctaatgctaaaaatccctacgttttactctatggcggtcaacatacccatgatttaagtcgccatcccaatgcttgtattgccatcaaaacagatgttaaccaagggcattgctccacagcggcaggacgttatcaatttttgactaagacttggcaggaaaaagcggctttgtatcaccctcaacgtcatttgggaaaatcacactataactttgagcctgaatttcaggatttagtaacctatcgatggttgaccgataaacaccactggggcatggacttttccacccaattacaacagggaaatatcgaacaagtgttgaaaaaactttctggcacttggacaagtttgggttacggcattgaagacaatagaatgaccgcttctttacccaaaatttatcaaaaactattagcagaagaacttgaccaagctaattaatattcgattcagtaccaagtactattgcggggacaggacgtttctcaaggccctcatcaatatcccccctgggggcatagaatagagatcaattttctaccccaaacccccacaatgggcaaactaccgcctatctttcactgcttgcggaaccgtctatttgctcagctatacctagcccaagccattagttccgcggaagggcgaattccacattgggctgcagcccgggggatccactagttctagagcggccgcaccgcgggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgattaaattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccttaatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttacagttaattaaagggaacaaaagctggcatgtaccgttcgtatagcatacattatacgaacggtacgctccaattcgccctttaattaactgttccaactttcaccataatgaaataagatcactaccgggcgtattttttgagttgtcgagattttcaggagctaaggaagctaaaatggagaaaaaaatcactggatataccaccgagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagct

SEQ ID NO: 10 - DNA, 6939 bp, Artificial Sequence
  PLASMID SEQUENCE BASED ON A VARIETY OF SOURCES
atgaccatgattacgccaagcgcgcaattaaccctcactaaagggaacaaaagctgggtaccgggccccccctcgaggtcgacggtatcgataagcttgatatccactgtggaattcgcccttggtaccgccaattgcagacgactacgggcaaagaggcgacgggtattcatggcgatagggtgaaccgatagccttgaccgggaactgttttaattgggcaaggacaattttgttgagctagcttgcgtcgtatcaaacgcatttgggccgccaccacattactcatgggctcctcatcaagatcccacagttgttgccggatcttgctaccggaaatgatccgctctgggttttgcatcagatattgaaaaatttgaaattctcttacggttaaagcaatttcctgtctttctaggtttagtggctccgagatagttaccgataacagattattactgggatcaaggctgaagttgcccaaagttaaaatttgcggttggaattgtggcgatcgccgttgtagtgcccgcagtcttgctaatagctctgccatcacaaacggttttgttagatagtcatctgccccggcatctagtccttcgacacggttttccggttctcctaacgctgttaacatcaacaccggcaaggaattaccctgggttctcagtttttgacagagttccaaacccgataatcccggcagtaaccaatccacaatggcaagggtgtattccgtccattgattttccaaataatcccaagcttgggagccatccgtcacccaatccaccacatacttttcactaactagcactttcttaatagccattcccaaatccgtctcatcttccaccagcaaaattcgcatcgcctctgccttttttataacggtctgatcttagcgggggaaggagattttcacctgaatttcataccccctttggcagactgggaaaatcttggacaaattcccaatttgaggtggtgtgatgccagaaaaacatgatctgttaaccgccatgatggcggcaaaggaacagggcatcggggcaatccttgcgtttgcaatggcgtaccttcgcggtcggtataatggcggtgcgtttaagaaaacactaatagacgcaacgatgtgcgccattatcgcctggttcattcgtgaccttttagtcttcgccggactgagtagcaatcttgcttacatagcgagtgtgtttatcggctacatcggcacagactcgattggttcgctaatcaaacgcttcgctgctaaaaaagccggagtcgatgatgcaaatcagcagtaacatatgaggaggtgtgatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatacatatgtctagacacattgctccttttgtgcgtaacgatagggtcagcactcaaaaatcgcatttttaaacgtgaattattatctcttctggctgtattaacaacggtgggagagatttttttaccacattttttctggttaatttccatgaccattacccaaaacacaactcgccactatcatcatcgtcgaaggtctcaacaatcatcttggtcacgccatttttttctggccactttattatttactctttgcttggcagcttttattagaaagtctcctgaaactgaaaacatcaattccttttttggccatttaccatccctagccatggaaggaggagatccttacattagagctttaatgcggacaatttcagccagtgaatctaatgctaaaaatccctacgttttactctatggcggtcaacatacccatgatttaagtcgccatcccaatgcttgtattgccatcaaaacagatgttaaccaagggcattgctccacagcggcaggacgttatcaatttttgactaagacttggcaggaaaaagcggctttgtatcaccctcaacgtcatttgggaaaatcacactataactttgagcctgaatttcaggatttagtaacctatcgatggttgaccgataaacaccactggggcatggacttttccacccaattacaacagggaaatatcgaacaagtgttgaaaaaactttctggcacttggacaagtttgggttacggcattgaagacaatagaatgaccgcttctttacccaaaatttatcaaaaactattagcagaagaacttgaccaagctaattaatattcgattcagtaccaagtactattgcggggacaggacgtttctcaaggccctcatcaatatcccccctgggggcatagaatagagatcaattttctaccccaaacccccacaatgggcaaactaccgcctatctttcactgcttgcggaaccgtctatttgctcagctatacctagcccaagccattagttccgcggaagggcgaattccacattgggctgcagcccgggggatccactagttctagagcggccgcaccgcgggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgattaaattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccttaatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttacagttaattaaagggaacaaaagctggcatgtaccgttcgtatagcatacattatacgaacggtacgctccaattcgccctttaattaactgttccaactttcaccataatgaaataagatcactaccgggcgtattttttgagttgtcgagattttcaggagctaaggaagctaaaatggagaaaaaaatcactggatataccaccgagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagct

SEQ ID NO: 11 - DNA, 8213 bp, Artificial Sequence
  PLASMID SEQUENCE BASED ON A VARIETY OF SOURCES
atgaccatgattacgccaagcgcgcaattaaccctcactaaagggaacaaaagctgggtaccgggccccccctcgaggtcgacggtatcgataagcttgatatccactgtggaattcgcccttggtaccgccaattgcagacgactacgggcaaagaggcgacgggtattcatggcgatagggtgaaccgatagccttgaccgggaactgttttaattgggcaaggacaattttgttgagctagcttgcgtcgtatcaaacgcatttgggccgccaccacattactcatgggctcctcatcaagatcccacagttgttgccggatcttgctaccggaaatgatccgctctgggttttgcatcagatattgaaaaatttgaaattctcttacggttaaagcaatttcctgtctttctaggtttagtggctccgagatagttaccgataacagattattactgggatcaaggctgaagttgcccaaagttaaaatttgcggttggaattgtggcgatcgccgttgtagtgcccgcagtcttgctaatagctctgccatcacaaacggttttgttagatagtcatctgccccggcatctagtccttcgacacggttttccggttctcctaacgctgttaacatcaacaccggcaaggaattaccctgggttctcagtttttgacagagttccaaacccgataatcccggcagtaaccaatccacaatggcaagggtgtattccgtccattgattttccaaataatcccaagcttgggagccatccgtcacccaatccaccacatacttttcactaactagcactttcttaatagccattcccaaatccgtctcatcttccaccagcaaaattcgcatcgcctctgccttttttataacggtctgatcttagcgggggaaggagattttcacctgaatttcataccccctttggcagactgggaaaatcttggacaaattcccaatttgaggtggtgtgatgccagaaaaacatgatctgttaaccgccatgatggcggcaaaggaacagggcatcggggcaatccttgcgtttgcaatggcgtaccttcgcggtcggtataatggcggtgcgtttaagaaaacactaatagacgcaacgatgtgcgccattatcgcctggttcattcgtgaccttttagtcttcgccggactgagtagcaatcttgcttacatagcgagtgtgtttatcggctacatcggcacagactcgattggttcgctaatcaaacgcttcgctgctaaaaaagccggagtcgatgatgcaaatcagcagtaacatatgaggaggtgtgatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatacatatgggatccttattttaagcactgactcctgatgtactcctgcaggtagttaacctgcgcggttatcctgtcgattccacttcggagacggtaataattgagttcagcatctgctgtaagtcttgggctttctccatcgcccatgccgctggctccggtcgttgactttggacaggtggcggagacttgcaggcgcttacgcccagcagaaacatcagcgcggagagtttcgatagtcgcgttagcatcagcaagctcctttgtgtatctggcatcgagttctgctacatctcgttgacgcttctgcatatcagcgatgattgatgtggctttatcgcgctgctctttgtaggtgatggcgttatcacgataatgattaacagcccacgaaagacagacgatgatgcagataatgacagacgcaataattgccttaatacgactcacgataagaacagcgctctttctcgccgcctccgtggaaggagaatatcagggtctttaccagcttttttccataacaggaaagcatctgctgctgcctggtaattctttaaattcaactggcgaagaacggtagagccggcaaatgctgatttacctatgttgaatatcaggctacatagcgcatcatactggttctgatttagcgggacgcgaacaagactacttatcgcatcttcaacccactgcaaatcctctttaagcagttcagaagatttttcggcggtgattgtcatccctgatgcgacagaattaccatccacttttccggtatgcccaaccccaatggttggtatccccctgctatctgaataggcttttagtctctcaccttcttcacgttttaatctggtgattccgttactgctgatttgcatcatttggttataattccttatgtatttgtcgatgttcagattggaactgactaaacttagtctaaaggattaatgagagttttgtaaagctttgtaacaggaagttaatatacacgaagcttatagatgacataagttttactttcttgtaattgtcgttttttccatgggtaaacagagggcgttttgggggttgagaagagtttacctggcggtttttcgtgcgcctttaccatagttctgggctgtgtagaggggcgacacaactgattttcagggacaatggagatgaaataatgaaaaataatgaaattgatcactgggccttgcacgccattactcttaaggtcagtggtcagtcagtttaagcaatagtcgggcatacaattaggatcctctagacacattgctccttttgtgcgtaacgatagggtcagcactcaaaaatcgcatttttaaacgtgaattattatctcttctggctgtattaacaacggtgggagagatttttttaccacattttttctggttaatttccatgaccattacccaaaacacaactcgccactatcatcatcgtcgaaggtctcaacaatcatcttggtcacgccatttttttctggccactttattatttactctttgcttggcagcttttattagaaagtctcctgaaactgaaaacatcaattccttttttggccatttaccatccctagccatggaaggaggagatccttacattagagctttaatgcggacaatttcagccagtgaatctaatgctaaaaatccctacgttttactctatggcggtcaacatacccatgatttaagtcgccatcccaatgcttgtattgccatcaaaacagatgttaaccaagggcattgctccacagcggcaggacgttatcaatttttgactaagacttggcaggaaaaagcggctttgtatcaccctcaacgtcatttgggaaaatcacactataactttgagcctgaatttcaggatttagtaacctatcgatggttgaccgataaacaccactggggcatggacttttccacccaattacaacagggaaatatcgaacaagtgttgaaaaaactttctggcacttggacaagtttgggttacggcattgaagacaatagaatgaccgcttctttacccaaaatttatcaaaaactattagcagaagaacttgaccaagctaattaatattcgattcagtaccaagtactattgcggggacaggacgtttctcaaggccctcatcaatatcccccctgggggcatagaatagagatcaattttctaccccaaacccccacaatgggcaaactaccgcctatctttcactgcttgcggaaccgtctatttgctcagctatacctagcccaagccattagttccgcggaagggcgaattccacattgggctgcagcccgggggatccactagttctagagcggccgcaccgcgggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgattaaattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccaccttaatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttacagttaattaaagggaacaaaagctggcatgtaccgttcgtatagcatacattatacgaacggtacgctccaattcgccctttaattaactgttccaactttcaccataatgaaataagatcactaccgggcgtattttttgagttgtcgagattttcaggagctaaggaagctaaaatggagaaaaaaatcactggatataccaccgagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcctttttttctgatcaaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagct

SEQ ID NO: 12 - DNA, 6939 bp, Artificial Sequence; identical to SEQ ID NO: 10
  PLASMID SEQUENCE BASED ON A VARIETY OF SOURCES

SEQ ID NO: 13 - DNA, 27 bp, Artificial Sequence
  PRIMER SEQUENCE BASED ON A VARIETY OF SOURCES
gcgagctccagacgactacgggcaaag

SEQ ID NO: 14 - DNA, 34 bp, Artificial Sequence
  PRIMER SEQUENCE BASED ON A VARIETY OF SOURCES
atgtttttctggcatcacaccacctcaaattggg

SEQ ID NO: 15 - DNA, 33 bp, Artificial Sequence
  PRIMER SEQUENCE BASED ON A VARIETY OF SOURCES
ttgaggtggtgtgatgccagaaaaacatgatct

SEQ ID NO: 16 - DNA, 28 bp, Artificial Sequence
  PRIMER SEQUENCE BASED ON A VARIETY OF SOURCES
gaccgcggttattttaagcactgactcc

SEQ ID NO: 17 - DNA, 27 bp, Artificial Sequence
  PRIMER SEQUENCE BASED ON A VARIETY OF SOURCES
ggccgcggaaagccacgttgtgtctca

SEQ ID NO: 18 - DNA, 33 bp, Artificial Sequence
  PRIMER SEQUENCE BASED ON A VARIETY OF SOURCES
accccctggggcagaaagccacgttgtgtctca

SEQ ID NO: 19 - DNA, 34 bp, Artificial Sequence
  PRIMER SEQUENCE BASED ON A VARIETY OF SOURCES
acaacgtggctttctgccccagggggtttcttga

SEQ ID NO: 20 - DNA, 27 bp, Artificial Sequence
  PRIMER SEQUENCE BASED ON A VARIETY OF SOURCES
gggatccgttggttagccaagagaatc

SEQ ID NO: 21 - DNA, 31 bp, Artificial Sequence
  PRIMER SEQUENCE BASED ON A VARIETY OF SOURCES
gacatatgttactgctgatttgcatcatcga

SEQ ID NO: 22 - DNA, 31 bp, Artificial Sequence
  PRIMER SEQUENCE BASED ON A VARIETY OF SOURCES
gatctagacacattgctccttttgtgcgtaa

SEQ ID NO: 23 - DNA, 32 bp, Artificial Sequence
  PRIMER SEQUENCE BASED ON A VARIETY OF SOURCES
gaccgcggaactaatggcttgggctaggtata

SEQ ID NO: 24 - DNA, 28 bp, Artificial Sequence
  PRIMER SEQUENCE BASED ON A VARIETY OF SOURCES
gaggtaccgccaattgcagacgactacg

SEQ ID NO: 25 - DNA, 31 bp, Artificial Sequence; identical to SEQ ID NO: 22
  PRIMER SEQUENCE BASED ON A VARIETY OF SOURCES

SEQ ID NO: 26 - DNA, 32 bp, Artificial Sequence; identical to SEQ ID NO: 23
  PRIMER SEQUENCE BASED ON A VARIETY OF SOURCES

SEQ ID NO: 27 - DNA, 25 bp, Artificial Sequence
  PRIMER SEQUENCE BASED ON A VARIETY OF SOURCES
aggcatgcgttggttagccaagaga

SEQ ID NO: 28 - DNA, 39 bp, Artificial Sequence
  PRIMER SEQUENCE BASED ON A VARIETY OF SOURCES
gcacaaaaggagcaatgtgttattttaagcactgactcc

SEQ ID NO: 29 - DNA, 36 bp, Artificial Sequence
  PRIMER SEQUENCE BASED ON A VARIETY OF SOURCES
tcagtgcttaaaataacacattgctccttttgtgcg

SEQ ID NO: 30 - DNA, 29 bp, Artificial Sequence
  PRIMER SEQUENCE BASED ON A VARIETY OF SOURCES
caaactaatggcttgggctaggtatagct

SEQ ID NO: 31 - DNA, 35 bp, Artificial Sequence
  PRIMER SEQUENCE BASED ON A VARIETY OF SOURCES
catgtttttctggcatcacaccacctcaaattggg

SEQ ID NO: 32 - DNA, 30 bp, Artificial Sequence
  PRIMER SEQUENCE BASED ON A VARIETY OF SOURCES
aggtggtgtgatgccagaaaaacatgacct

SEQ ID NO: 33 - DNA, 39 bp, Artificial Sequence
  PRIMER SEQUENCE BASED ON A VARIETY OF SOURCES
acaaaaggagcaatgtgctatctgcactgctcattaata

SEQ ID NO: 34 - DNA, 32 bp, Artificial Sequence
  PRIMER SEQUENCE BASED ON A VARIETY OF SOURCES
agtgcagatagcacattgctccttttgtgcgt

SEQ ID NO: 35 - DNA, 32 bp, Artificial Sequence; identical to SEQ ID NO: 23
  PRIMER SEQUENCE BASED ON A VARIETY OF SOURCES

SEQ ID NO: 36 - DNA, 68 bp, Artificial Sequence
  PRIMER SEQUENCE BASED ON A VAREITY OF SOURCES
atcatatgaagacaaacgaaagcccccacctagcgtcatgccgggtgggggctttttcatctgcagta

SEQ ID NO: 37 - DNA, 68 bp, Artificial Sequence
  PRIMER SEQUENCE BASED ON A VAREITY OF SOURCES
tactgcagatgaaaaagcccccacccggcatgacgctaggtgggggctttcgtttgtcttcatatgat

SEQ ID NO: 38 - DNA, 23 bp, Artificial Sequence
  PRIMER SEQUENCE BASED ON A VARIETY OF SOURCES
ctgcagatgaaaaagcccccacc

SEQ ID NO: 39 - DNA, 28 bp, Artificial Sequence
  PRIMER SEQUENCE BASED ON A VARIETY OF SOURCES
gaggatcctaattgtatgcccgactatt

SEQ ID NO: 40 - DNA, 37 bp, Artificial Sequence
  PRIMER SEQUENCE BASED ON A VARIETY OF SOURCES
actgctgatttgcatcatttggttataattccttatg

SEQ ID NO: 41 - DNA, 36 bp, Artificial Sequence
  PRIMER SEQUENCE BASED ON A VARIETY OF SOURCES
gaattataaccaaatgatgcaaatcagcagtaacgg

SEQ ID NO: 42 - DNA, 29 bp, Artificial Sequence
  PRIMER SEQUENCE BASED ON A VARIETY OF SOURCES
gaggatccttattttaagcactgactcct

SEQ ID NO: 43 - DNA, 27 bp, Artificial Sequence
  PRIMER SEQUENCE BASED ON A VARIETY OF SOURCES
gacatatgccagaaaaacatgacctgt

SEQ ID NO: 44 - DNA, 26 bp, Artificial Sequence
  PRIMER SEQUENCE BASED ON A VARIETY OF SOURCES
agaagctttgtggcccaacaattggt

SEQ ID NO: 45 - DNA, 29 bp, Artificial Sequence
  PRIMER SEQUENCE BASED ON A VARIETY OF SOURCES
gtgaattctgtaagcagttagagtggccc

SEQ ID NO: 46 - DNA, 28 bp, Artificial Sequence
  PRIMER SEQUENCE BASED ON A VARIETY OF SOURCES
cggtctactccggttaaatcccctaacg

SEQ ID NO: 47 - DNA, 25 bp, Artificial Sequence
  PRIMER SEQUENCE BASED ON A VARIETY OF SOURCES
ccacagccccaacaataagcaagat

SEQ ID NO: 48 - DNA, 27 bp, Artificial Sequence
  PRIMER SEQUENCE BASED ON A VARIETY OF SOURCES
gacatatgccagaaaaacatgacctgt

SEQ ID NO: 49 - DNA, 37 bp, Artificial Sequence
  PRIMER SEQUENCE BASED ON A VARIETY OF SOURCES
gacatatgaggaggtgtgatgccagaaaaacatgacc

SEQ ID NO: 50 - DNA, 37 bp, Artificial Sequence; identical to SEQ ID NO: 40
  PRIMER SEQUENCE BASED ON A VARIETY OF SOURCES

SEQ ID NO: 51 - DNA, 36 bp, Artificial Sequence; identical to SEQ ID NO: 41
  PRIMER SEQUENCE BASED ON A VARIETY OF SOURCES

(19) United States
(12) Patent Application Publication
(10) Pub. No.: US 2011/0159594 A1
(43) Pub. Date: Jun. 30, 2011

(54) NUCLEIC ACIDS, BACTERIA, AND METHODS FOR DEGRADING THE PEPTIDOGLYCAN LAYER OF A CELL WALL

(75) Inventors: Roy Curtiss, III, Paradise Valley, AZ (US); Xinyao Liu, Tempe, AZ (US)

(73) Assignee: The Arizona Board of Regents for and on behalf of Arizona State University, Tempe, AZ (US)

(21) Appl. No.: 12/999,840

(22) PCT Filed: Jun. 17, 2009
(86) PCT No.: PCT/US09/47681

§ 371 Date: Mar. 15, 2011

Related U.S. Application Data
(60) Provisional application No. 61/073,299, filed on Jun. 17, 2008.

(51) Int. Cl. C12N 15/63; C12N 15/74; C12N 1/21

(52) U.S. Cl. 435/471; 435/252.3

(57) ABSTRACT

The invention encompasses compositions and methods for degrading the peptidoglycan layer of a cell wall. In particular, the invention encompasses compositions and methods for degrading the peptidoglycan layer of the cell wall of a gram-negative bacterium.

### CROSS REFERENCE TO RELATED APPLICATIONS

This application claims the priority of U.S. provisional application No. 61/073,299, filed Jun. 17, 2008, which is hereby incorporated by reference in its entirety.

### FIELD OF THE INVENTION

The invention encompasses compositions and methods for degrading the peptidoglycan layer of a cell wall.

### BACKGROUND OF THE INVENTION

With the development of bacterial genetics, many bacteria have been genetically designed as bioreactors to produce numerous products of value, such as proteins, chemicals, drugs, and fuels. Generally, most of the valuable products are produced and accumulated inside the bacterial cells. After fermentation, the bacterial cell wall needs to be disrupted in order to facilitate product recovery from the bacterial biomass. The traditional cell processing techniques include physical or chemical cell breakage methods such as sonication, homogenization, pressure decompression, addition of hydrolytic enzymes and by solvent disruption and extraction. However, most of these methods require high energy inputs or raise environmental issues that reduce the overall utility of the process.

A bacterial cell wall is comprised, in part, of peptidoglycan (also called murein) made from polysaccharide chains cross-linked by unusual peptides containing D-amino acids. The efficient release of the cytoplasmic contents of a bacterial cell depends in part on the degradation of the peptidoglycan layer of the cell wall. Such degradation is preferably regulated, so that the timing can be controlled. Consequently, there is a need in the art for efficient and regulable methods to degrade the peptidoglycan layer of bacterial cell walls to release products accumulated within the cell.

### SUMMARY OF THE INVENTION

One aspect of the present invention encompasses a method for degrading the peptidoglycan layer of the cell wall of a gram-negative bacterium. The method typically comprises introducing into the bacterium a nucleic acid comprising an inducible promoter operably-linked to a nucleic acid. The nucleic acid encodes a first protein capable of forming a lesion in the cytoplasmic membrane of the bacterium and at least one endolysin protein. The method further comprises inducing the promoter to express both the first protein and the endolysin, wherein the first protein allows the endolysin to degrade the peptidoglycan layer of the cell wall.

Another aspect of the present invention encompasses a method for degrading the peptidoglycan layer of the cell wall of a gram-negative bacterium. The method generally comprises introducing into the bacterium a first nucleic acid comprising a first inducible promoter operably-linked to a nucleic acid. The nucleic acid encodes a first protein capable of forming a lesion in the cytoplasmic membrane of the bacterium. The method further comprises introducing into the bacterium a second nucleic acid comprising a second promoter operably-linked to at least one endolysin protein. The inducible promoter is induced so as to express the first protein wherein the first protein allows the endolysin to degrade the peptidoglycan layer of the cell wall.

Yet another aspect of the present invention encompasses a gram-negative bacterium. The bacterium comprises a first nucleic acid, wherein the first nucleic acid comprises a first inducible promoter operably-linked to a nucleic acid encoding a first protein capable of forming a lesion in the cytoplasmic membrane of the bacterium. The bacterium also comprises a second nucleic acid, wherein the second nucleic acid comprises a second promoter operably-linked to a nucleic acid encoding at least one endolysin protein.

Still another aspect of the present invention encompasses a nucleic acid comprising a first inducible promoter operably-linked to a nucleic acid encoding a first protein capable of forming a lesion in the cytoplasmic membrane of the bacterium and a second promoter operably-linked to a nucleic acid encoding at least one endolysin protein.

Other aspects and iterations of the invention are described more thoroughly below.

### BRIEF DESCRIPTION OF THE FIGURES

FIG. 1 depicts an illustration of the construction of suicide vector pψ101. f1 and f2 are right and left flanking DNA respectively for double crossover recombination that were amplified from Synechocystis genome DNA. The f1 sequence contains the Synechocystis nsrRS genes and the Ni²⁺ inducible promoter. 13, 19, and 15 in the rightward arrow boxes refer to the lysis genes 13, 19 and 15 from the Salmonella phage P22 genome, which were amplified from a P22 lysate using PCR. The Km^{r} in the leftward arrow box refers to the kanamycin resistance cassette, which was amplified from plasmid pUC4K. Using overlapping PCR and ligation. These DNA fragments were inserted into a cloning vector pSC-A giving the resultant suicide vector pψ101.

FIG. 2 depicts a picture (A) and a graph (B) of the Ni²⁺ induced lysis of Synechocystis recombinant SD101 after Ni²⁺ addition. The picture (A) shows that after Ni²⁺ addition, the Synechocystis cells in the liquid cultures were lysed. The graph (B) shows that at the absorbance (730 nm) the strain SD101 declined significantly in the presence of different concentrations of Ni²⁺ (3.5, 7 and 17 μM).

FIG. 3 depicts the methods for introducing lysis genes into Synechocystis constructions. Step 1: Transforming wild-type Synechocystis cells with a suicide vector pψ102 containing Km^{R}-sacB; Step 2: Selecting for kanamycin resistance for the intermediate strain SD102; Step 3: Transforming SD102 with a markerless suicide vector, pψLYS containing lysis genes; Step 4: Selecting the right insertions SD1XX on sucrose plates. Abbreviations: f1 and f2, flanking regions, which are partial sequences of Synechocystis nrsSR and nrsD, respectively; Km^{R}, kanamycin resistance cassette; sacB, sacB gene, which is lethal for cyanobacteria in the presence of sucrose; LYS represents the lysis gene cassette.

FIG. 4. depicts the strains and strategies used in this study. nrsRS, nickel sensing and responding genes; P_{nrsB}, the nickel inducible promoter; nrsBACD, nickel resistance genes; 13, 19 and 15, Salmonella phage P22 genes 13 (holin), 19 (endolysin) and 15; S, R and Rz, coliphage λ genes S (holin), R (endolysin) and Rz; Km^{R}, kanamycin resistance cassette; sacB, sacB gene, which is lethal for cyanobacteria in the presence of sucrose; P_{psbAll}, promoter of Synechocystis gene psbAll; TP4, transcriptional terminator from cyanophage Pf-WMP4.

FIG. 5 depicts PCR identification of the absence of replaced regions in SD strains. The primers specific for the original Synechocystis nrsBA region were used; unmarked lanes were used for another project.

FIG. 6 depicts PCR identification of the replacement of sacB in SD strains. The primers specific for the sacB gene were used; unmarked lanes were used for another project.

FIG. 7 depicts PCR identification of holin gene 13 and P_{psbAll} 15 19 cassette in SD strains. Left side, the primers specific for P22 holin gene 13 were used; right side, the primer specific for the whole insertion region was used. Plasmid pψ123 was used as a positive control.

FIG. 8 depicts PCR identification of P_{psbAll} 15 19 cassette in SD123, 124 and 127 strains over a 60-generation continuous culture. Plasmid pψ123 was used as a positive control. The cultures of SD123, 124 and 127 were grown from single colonies. 15G, 30G, 45G, and 60G indicate the cultures were sampled at around 15, 30, 45, and 60 generations of growth.

FIG. 9 depicts the frequencies of Ni²⁺ mutants for the Ni²⁺ inducible lysis strains as a function of number of generations of growth.

FIG. 10 depicts the semi-log growth curves for recombinant and wild type strains. The growth rates of SD strains were calculated from the slope during the exponential growth stage.

FIG. 11 depicts the lysis rates of SD123 at different Ni²⁺ concentrations. Lysis rates were calculated as the decrease in percentage of viable cell titers per hour after Ni²⁺ was added to SD123 cultures at final concentrations of 1, 3, 7, 20, 5 and100 μM.

FIG. 12 depicts the induced lysis of SD strains after addition of 7.0 μM NiSO₄. The vital cell titers of different time points after Ni²⁺ addition were measured by colony formation units on BG-11 plates.

FIG. 13 depicts the induced lysis of SD strains after addition of 20 mM (A) and 50mM NiSO₄ (B).

FIG. 14 depicts fluorescence images of SD123 cells stained with SYTOX Green dye after addition of 7 μM Ni²⁺. The samples were stained with SYTOX Green and inspected under a fluorescence microscope before and 3, 6, and 9 hours after the addition of 7 μM Ni²⁺ to a SD123 culture. Green fluorescence indicated the penetrable lysing cells, and red auto fluorescence indicated the intact viable cells.

FIG. 15 depicts penetration rates of SD strains by SYTOX Green after 7 μM Ni²⁺ addition. The penetrable cell ratio of lysing cultures after 7 μM Ni²⁺ addition were counted as the percentage of green cells in a total of at least 400 cells (green plus red).

FIG. 16 depicts TEM images of the SD121 cells before and after the addition of 7 μM of Ni²⁺. (A), SD121 cells before Ni²⁺ addition; (B), 6 hr after Ni²⁺ addition; (C), 12 hr after Ni²⁺ addition; (D), 24 hr after Ni²⁺ addition.

FIG. 17 depicts the sequence of pSC-A. (SEQ ID NO:1)

FIG. 18 depicts the sequence of pPsbA2KS. (SEQ ID NO:2)

FIG. 19 depicts the sequence of pψ101. (SEQ ID NO:3)

FIG. 20 depicts the sequence of pψ102. (SEQ ID NO:4)

FIG. 21 depicts the sequence of pψ103. (SEQ ID NO:5)

FIG. 22 depicts the sequence of pψ121. (SEQ ID NO:6)

FIG. 23 depicts the sequence of pψ122. (SEQ ID NO:7)

FIG. 24 depicts the sequence of pψ123. (SEQ ID NO:8)

FIG. 25 depicts the sequence of pψ124. (SEQ ID NO:9)

FIG. 26 depicts the sequence of pψ125. (SEQ ID NO:10)

FIG. 27 depicts the sequence of pψ126. (SEQ ID NO:11)

FIG. 28 depicts the sequence of pψ127. (SEQ ID NO:12)

### DETAILED DESCRIPTION OF THE INVENTION

The present invention provides a method for inducing the degradation of the peptidoglycan layer of a gram-negative bacterial cell wall. In particular, it has been discovered that the regulated expression of a protein capable of forming a lesion in the cytoplasmic membrane may be used to allow at least one endolysin to degrade the peptidoglycan layer of a bacterial cell wall. The invention also provides nucleic acid constructs comprising a nucleic acid encoding a protein capable of forming a lesion in the cytoplasmic membrane and at least one endolysin. Additionally, the invention encompasses a bacterium comprising a nucleic acid construct of the invention.

### I. Nucleic Acid Constructs

One aspect of the present invention encompasses a nucleic acid construct that, when introduced into a bacterium, may be used in a method for inducing the degradation of the peptidoglycan layer of a bacterial cell wall. In one embodiment, the nucleic acid comprises an inducible promoter operably-linked to a nucleic acid sequence encoding a first protein capable of forming a lesion in a bacterial cytoplasmic membrane. In another embodiment, the nucleic acid comprises an inducible promoter operably-linked to both a nucleic acid sequence encoding a first protein and a nucleic acid sequence encoding at least one endolysin. In yet another embodiment, the nucleic acid comprises a promoter operably-linked to at least one endolysin encoding sequence. In still another embodiment, the nucleic acid comprises an inducible promoter operably-linked to a nucleic acid sequence encoding a first protein and a second promoter operably-linked to a nucleic acid sequence encoding at least one endolysin. In certain embodiments, the invention encompasses nucleic acid constructs illustrated in FIG. 4 and delineated in Table A. Each component of the above nucleic acid constructs is discussed in more detail below.

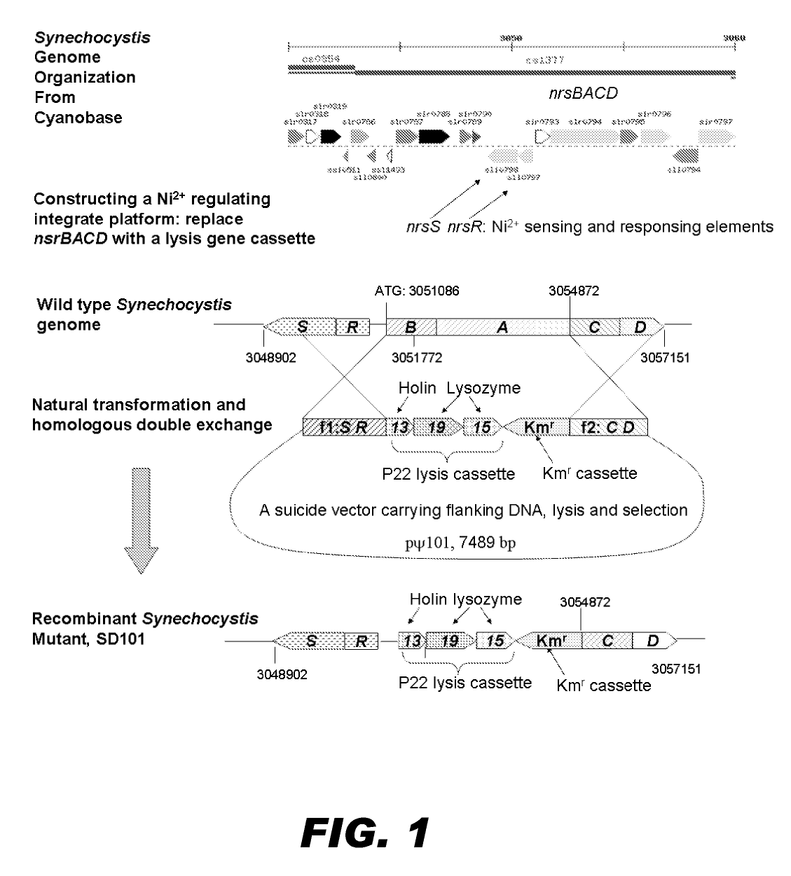

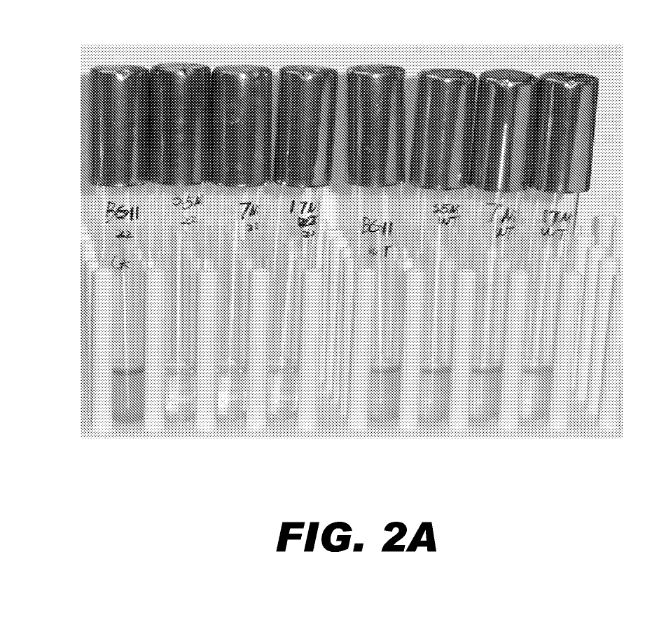

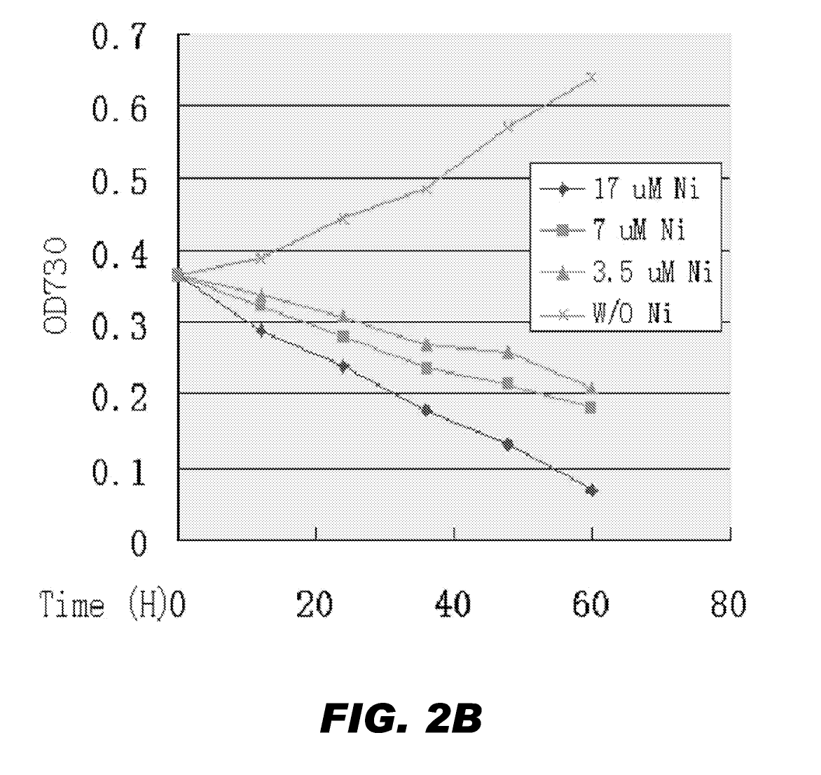

Methods of making a nucleic acid construct of the invention are known in the art. For more details, see the figure legends for FIGS. 1, 3, and 4, or the Examples. Additional information may be found in Sambrook et al., Molecular Cloning: A Laboratory Manual (New York: Cold Spring Harbor Laboratory Press, 1989)

### (a) Promoters

A nucleic acid construct of the present invention comprises a promoter. In particular, a nucleic acid construct comprises a first inducible promoter. In some embodiments, a nucleic acid also comprises a second promoter. When a nucleic acid comprises a first and a second promoter, the promoters may read in opposite directions, or may read in the same direction. For instance, see FIG. 4, SD123 & SD124.

i. First Inducible Promoter

In certain embodiments, a nucleic acid of the invention encompasses a first inducible promoter. Non-limiting examples of inducible promoters may include, but are not limited to, those induced by expression of an exogenous protein (e.g., T7 RNA polymerase, SP6 RNA polymerase), by the presence of a small molecule (e.g., IPTG, galactose, tetracycline, steroid hormone, abscisic acid), by metals or metal ions (e.g., copper, zinc, cadmium, nickel), and by environmental factors (e.g., heat, cold, stress). In each of the above embodiments, the inducible promoter is preferably tightly regulated such that in the absence of induction, substantially no transcription is initiated through the promoter. Additionally, induction of the promoter of interest should not typically alter transcription through other promoters. Also, generally speaking, the compound or condition that induces an inducible promoter should not be naturally present in the organism or environment where expression is sought.

In one embodiment, the inducible promoter is induced by a metal or metal ion. By way of non-limiting example, the inducible promoter may be induced by copper, zinc, cadmium, mercury, nickel, gold, silver, cobalt, and bismuth or ions thereof. In one embodiment, the inducible promoter is induced by nickel or a nickel ion. In an exemplary embodiment, the inducible promoter is induced by a nickel ion, such as Ni²⁺. In another exemplary embodiment, the inducible promoter is the nickel inducible promoter from Synechocystis PCC6803. In another embodiment, the inducible promoter may be induced by copper or a copper ion. In yet another embodiment, the inducible promoter may be induced by zinc or a zinc ion. In still another embodiment, the inducible promoter may be induced by cadmium or a cadmium ion. In yet still another embodiment, the inducible promoter may be induced by mercury or a mercury ion. In an alternative embodiment, the inducible promoter may be induced by gold or a gold ion. In another alternative embodiment, the inducible promoter may be induced by silver or a silver ion. In yet another alternative embodiment, the inducible promoter may be induced by cobalt or a cobalt ion. In still another alternative embodiment, the inducible promoter may be induced by bismuth or a bismuth ion.

In some embodiments, the promoter is induced by exposing a cell comprising the inducible promoter to a metal or metal ion. The cell may be exposed to the metal or metal ion by adding the metal to the bacterial growth media. In certain embodiments, the metal or metal ion added to the bacterial growth media may be efficiently recovered from the media. In other embodiments, the metal or metal ion remaining in the media after recovery does not substantially impede downstream processing of the media or of the bacterial gene products.

In one embodiment, the nucleic acid comprises a metal or metal ion inducible promoter operably-linked to a nucleic acid sequence encoding a first protein capable of forming a lesion in a bacterial cytoplasmic membrane. In another embodiment, the nucleic acid comprises a metal or metal ion inducible promoter operably-linked to both a nucleic acid sequence encoding a first protein and a nucleic acid sequence encoding at least one endolysin. In yet another embodiment, the nucleic acid comprises a metal or metal ion inducible promoter operably-linked to at least one endolysin. In still another embodiment, the nucleic acid comprises a metal or metal ion inducible promoter operably-linked to a nucleic acid sequence encoding a first protein and a second promoter operably-linked to a nucleic acid sequence encoding at least one endolysin.

ii. Second Promoter

Certain nucleic acid constructs of the invention may comprise a second promoter. The second promoter may be an inducible promoter, or may be a constitutive promoter. If the second promoter is an inducible promoter, it may or may not be induced by the same compound or condition that induces the first inducible promoter. In one embodiment, the same compound or condition induces both the first and the second inducible promoters. In another embodiment, the first inducible promoter is induced by a different compound or condition than the second inducible promoter. Non-limiting examples of inducible promoters that may be used are detailed in section I(a)(i) above.

Constitutive promoters that may comprise the second promoter are known in the art. Non-limiting examples of constitutive promoters may include constitutive promoters from Gram negative bacteria or a Gram negative bacteriophage. For instance, promoters from highly expressed Gram negative gene products may be used, such as the promoter for Lpp, OmpA, rRNA, and ribosomal proteins. Alternatively, regulatable promoters may be used in a strain that lacks the regulatory protein for that promoter. For instance P_{lac}, P_{tac}, and P_{trc} may be used as constitutive promoters in strains that lack Lacl. Similarly, P22 P_{R} and P_{L} may be used in strains that lack the P22 C2 repressor protein, and λ P_{R} and P_{L} may be used in strains that lack the λ C1 repressor protein. In one embodiment, the constitutive promoter is from a bacteriophage. In another embodiment, the constitutive promoter is from a Salmonella bacteriophage. In yet another embodiment, the constitutive promoter is from a cyanophage. In some embodiments, the constitute promoter is a Synechocystis promoter. For instance, the constitutive promoter may be the P_{psbAll} promoter.

In one embodiment, a nucleic acid of the invention comprises a metal or metal ion inducible promoter operably-linked to a nucleic acid sequence encoding a first protein and a second constitutive promoter operably-linked to a nucleic acid sequence encoding at least one endolysin. In another embodiment, a nucleic acid of the invention comprises a metal or metal ion inducible promoter operably-linked to a nucleic acid sequence encoding a first protein and a second inducible promoter operably-linked to a nucleic acid sequence encoding at least one endolysin.

### (b) First Protein

A nucleic acid construct of the invention also comprises a sequence encoding at least one first protein. Generally speaking, a first protein is a protein capable of forming a lesion in the cytoplasmic membrane that provides the endolysin access to the peptidoglycan layer of the cell wall. In some embodiments, the first protein is a bacteriophage protein. For instance, the first protein may be a bacteriophage holin protein. In one embodiment, the first protein is a holin from a bacteriophage that infects gram-negative bacteria. In another embodiment, the first protein is a holin from a bacteriophage that infects gram-positive bacteria. In certain embodiments, the first protein is a holin from a cyanophage. In one embodiment, the first protein is a holin from a bacteriophage that infects Synechocystis. In another embodiment, the first protein may be from a bacteriophage that infects Salmonella. In still another embodiment, the first protein may be from a P22 phage. For example, the first protein may be gene 13 of the P22 phage. In yet another embodiment, the first protein may be from a λ phage. For example, the first protein may be encoded by gene S of the λ phage. In still another embodiment, the first protein may be from an E. coli phage. For instance, the first protein may be encoded by gene E of E. coli phage PhiX174. In certain embodiments, a nucleic acid of the invention may comprise at least two holins. In one embodiment, a nucleic acid may comprise a holin from P22 and a holin from λ phage. For instance, the nucleic acid may comprise gene 13 and gene S.

Non-limiting examples of bacteriophages that may encode suitable holin proteins include phages of Actinomycetes, such as A1-Dat, Bir, M1, MSPS, P-a-1, R1, R2, SV2, VPS, PhiC, ⊥31C, ⊥UW21, ⊥115-A, ⊥150A, 119, SK1, and 108/016; phages of Aeromonas, such as 29, 37, 43, 51, and 59.1; phages of Altermonas, such as PM2; phages of Bacillus, such as APS, ⊥NS11, BLE, Ipy-1, MP15, mor1, PBP1, SPP1, Spbb, type F, alpha, ⊥105, 1A, II, Spy-2, SST, G, MP13, PBS1, SP3, SP8, SP10, SP15, and SP50; phages of Bdellovibrio, such as MAC-1, MAC-1′, MAC-2, MAC-4, MAC-4′, MAC-5, and MAC-7; phages of Caulobacter, such as ⊥Cb2, ⊥Cb4, ⊥Cb5, ⊥Cb8r, ⊥Cb9, ⊥CB12r, ⊥Cb23r, ⊥CP2, ⊥CP18, ⊥Cr14, ⊥Cr28, PP7, ⊥Cb2, ⊥Cb4, ⊥Cb5, ⊥Cb8r, ⊥Cb9, ⊥CB12r, ⊥Cb23r, ⊥CP2, ⊥CP18, ⊥Cr14, ⊥Cr28, and PP7; phages of Chlamydia such as Chp-1; phages of Clostridium, such as F1, HM7, HM3, CEB; phages of Coryneforms, such as Arp, BL3, CONX, MT, Beta, A8010, and A19; phages of Enterobacter, such as C-2, If1, If2, Ike, I 2-2, PR64FS, SF, tf-1, PRD1, H-19J, B6, B7, C-1, C2, Jersey, ZG/3A, T5, ViII, b4, chi, Beccles, tu, PRR1, 7s, C-1, c2, fcan, folac, lalpha, M, pilhalpha, R23, R34, ZG/1, ZIK/1, ZJ/1, ZL/3, ZS/3, alpha15, f2, fr, FC3-9, K19, Mu, 01, P2, ViI, 192, 121, 16-19, 9266, C16, DdVI, PST, SMB, SMP2, a1, 3, 3T+, 9/0, 11 F, 50, 66F, 5845, 8893, M11, QB, ST, TW18, VK, FI, ID2, fr, and f2; phages of Listeria, such as H387, 2389, 2671, 2685, and 4211; phages of Micrococcus such as N1 and N5; phages of Mycobacterium, such as Lacticola, Leo, R1-Myb, and 13; phages of Pasteurella, such as C-2, 32, and AU; phages of Pseudomonas such as Phi6, Pf1, Pf2, Pf3, D3, Kf1, M6, PS4, SD1, PB-1, PP8, PS17, nKZ, nW-14, n1, and 12S; phages of Staphyloccous, such as 3A, B11-M15, 77, 107, 187, 2848A, and Twort; phages of Streptococcus, such as A25, A25 PE1, A25 VD13, A25 omega8, A25, and 24; phages of Steptococcus A, such as OXN-52P, VP-3, VP5, VP11, alpha3alpha, IV, and kappa; phages of Vibrio, such as 06N-22-P, VP1, x29, II, and nt-1; and phages of Xanthomonas, such as Cf, Cf1t, Xf, Xf2, and XP5. Non-limiting examples of phages of Cyanobacteria that may encode suitable holins include S-2L, S-4L, N1, AS-1, S-6(L), AN-10, AN-15, A-1(L), A-2, NN-Anabaena, AS-1M, NN-Anacystis, NN-Plectonema, S-BM1, S-BS1, S-PM1, S-PS1, S-PWM, S-PWM1, S-PWM2, S-PMW4, S-WHM1, S-3(L), S-7(L), NN-Synechococcus, AC-1, AN-20, AN-22, AN-24, A-4(L), AT, GM, GIII, LPP-1, SPI, WA S-BBP1, S-PWP1, SM-1, S-5(L), NN-Phormidium, S-BBS1, S-BBS1, SM-2, and S-1.

Additionally, a first protein may be a holin described above with at least one, or a combination of one or more, nucleic acid deletions, substitutions, additions, or insertions which result in an alteration in the corresponding amino acid sequence of the encoded holin protein, such as a homolog, ortholog, mimic or degenerative variant. For instance, a first protein may be a holin described above encoded by a nucleic acid with codons optimized for use in a particular bacterial strain, such as Synechocystis. Such a holin may be generated using recombinant techniques such as site-directed mutagenesis (Smith Annu. Rev. Genet. 19. 423 (1985)), e.g., using nucleic acid amplification techniques such as PCR (Zhao et al. Methods Enzymol. 217, 218 (1993)) to introduce deletions, insertions and point mutations. Other methods for deletion mutagenesis involve, for example, the use of either BAL 31 nuclease, which progressively shortens a double-stranded DNA fragment from both the 5′ and 3′ ends, or exonuclease III, which digests the target DNA from the 3′end (see, e. g., Henikoff Gene 28, 351 (1984)). The extent of digestion in both cases is controlled by incubation time or the temperature of the reaction or both. Point mutations can be introduced by treatment with mutagens, such as sodium bisulfite (Botstein et al. Science 229, 1193 (1985)). Other exemplary methods for introducing point mutations involve enzymatic incorporation of nucleotide analogs or misincorporation of normal nucleotides or alpha-thionucleotide by DNA polymerases (Shortle et al. Proc. Natl. Acad. Sci. USA79,1588 (1982)). PCR-based mutagenesis methods (or other mutagenesis methods based on nucleic acid amplification techniques), are generally preferred as they are simple and more rapid than classical techniques (Higuchi et al. Nucleic Acids Res. 16, 7351 (1988); Vallette et al. Nucleic Acids Res. 17,723 (1989)).

In addition to having a substantially similar biological function, a homolog, ortholog, mimic or degenerative variant of a holin suitable for use in the invention will also typically share substantial sequence similarity to a holin protein. In addition, suitable homologs, orthologs, mimics or degenerative variants preferably share at least 30% sequence homology with a holin protein, more preferably, 50%, and even more preferably, are greater than about 75% homologous in sequence to a holin protein. Alternatively, peptide mimics of a holin could be used that retain critical molecular recognition elements, although peptide bonds, side chain structures, chiral centers and other features of the parental active protein sequence may be replaced by chemical entities that are not native to the holin protein yet, nevertheless, confer activity.

In determining whether a polypeptide is substantially homologous to a holin polypeptide, sequence similarity may be determined by conventional algorithms, which typically allow introduction of a small number of gaps in order to achieve the best fit. In particular, “percent homology” of two polypeptides or two nucleic acid sequences is determined using the algorithm of Karlin and Altschul [(Proc. Natl. Acad. Sci. USA 87, 2264 (1993)]. Such an algorithm is incorporated into the NBLAST and XBLAST programs of Altschul, et al. (J. Mol. Biol. 215, 403 (1990)). BLAST nucleotide searches may be performed with the NBLAST program to obtain nucleotide sequences homologous to a nucleic acid molecule of the invention. Equally, BLAST protein searches may be performed with the XBLAST program to obtain amino acid sequences that are homologous to a polypeptide of the invention. To obtain gapped alignments for comparison purposes, Gapped BLAST is utilized as described in Altschul, et al. (Nucleic Acids Res. 25, 3389 (1997)). When utilizing BLAST and Gapped BLAST programs, the default parameters of the respective programs (e.g., XBLAST and NBLAST) are employed. See http://www.ncbi.nlm.nih.gov for more details.

In one embodiment, a nucleic acid of the invention comprises a metal or metal ion inducible promoter operably-linked to a nucleic acid sequence encoding a P22 phage holin. In another embodiment, the nucleic acid comprises a metal or metal ion inducible promoter operably-linked to both a nucleic acid sequence encoding a P22 phage holin and a nucleic acid sequence encoding at least one endolysin. In yet another embodiment, the nucleic acid comprises a metal or metal ion inducible promoter operably-linked to a nucleic acid sequence encoding a P22 phage holin and a second promoter operably-linked to a nucleic acid sequence encoding at least one endolysin.

### (c) Endolysin

In some embodiments, a nucleic acid of the invention comprises at least one endolysin. In other embodiments, a nucleic acid of the invention comprises at least two endolysins. In yet another embodiment, a nucleic acid of the invention comprises at least three endolysins. In still another embodiment, a nucleic acid of the invention may comprise at least four endolysins. As used herein, “endolysin” refers to a protein capable of degrading the peptidoglycan layer of a bacterial cell wall. Generally speaking, the term endolysin encompasses proteins selected from the group consisting of lysozyme or muramidase, glucosaminidase, transglycosylase, amidase, and endopeptidase. Exemplary endolysins do not affect the cell until after the first protein creates lesions in the cytoplasmic membrane. Stated another way, the accumulation of endolysins in the cytosol of a bacterium will typically not substantially impair the growth rate of the bacterium. In another exemplary embodiment, the endolysin has a high enzymatic turnover rate. In yet another exemplary embodiment, the endolysin is from a gram positive bacteria. Because the cell walls of gram positive bacteria typically have a thicker peptidoglycan layer, an endolysin from a gram positive bacteria might be expected to have a higher enzymatic turnover rate.

Non-limiting examples of endolysins that may be suitable include the canonical lysozyme T4 gpe (GI126605), the P22 endolysin gp19 (GI963553), Lys of phage Mu (GI9633512), Lys of Haemophilus influenzae phage HP1 (GI1708889), Lyz of Erwinia amylovora phage phiEA1 H (GI11342495), gp45 of Pseudomonas aeruginosa phage KMV, R21 of lambdoid phage 21 (GI126600), gp19 of Salmonella typhimurium phage PS34 (GI3676081), muramidase and endopeptidase of Streptococcus agalactiae bacteriophage B30, endopeptidase and amidase of Staphylococcus aureus phage 11, endopeptidase and muramidase of S. agalactiae phage NCTC 11261, endopeptidase and amidase of Staphylococcus warneri M phage WMY, Lys44 from Oenococcus oeni phage fOg44, Lyz from coliphage P1, Lys from Lactobacillus plantarum phage g1e, PlyV12 from Enterococcus faecalis phage 1, Mur-LH of Lactobacillus helveticus phage-0303, endolysin derived from the Bacillus amyloliquefaciens phage, auxiliary endolysin lys1521 from Bacillus amyloliquefaciens phage, C-truncated Mur from Lactobacillus delbrueckii phage LL-H, Ply511 lysin from L. monocytogenes phage A511, PIyL from Bacillus anthracis prophage Ba02, Ply21 from B. cereus phage TP21, Plyl18 from L. monocytogenes phages A118, Ply500 from L. monocytogenes phages A500, Ply3626 from C. perfringens phage 3626, endolysin from Group C streptococci C1 phage, Pal amidase from phage Dp-1, Cpl-1 lysozyme from Cp-1 phage, PIyGBS from S. agalactiae phage NCTC 11261, amidase from B. anthracis phage PIyG, LysA an endolysin of Lactobacillus delbrueckii subsp. bulgaricus bacteriophage mv1, VG14_BPB03 from bacteriophage B103, VG14_BPPZA from bacteriophage PZA, G14_BPPH2 from bacteriophage Ø-29, ESSD_ECOLI from prophage DLP12, VLYS_BPP21 from bacteriophage 21, VLYS_BPAPS from bacteriophage APSE-1, VLY1_BPP22 from bacteriophage P22, T4, T7, and lamda R. Also included are the chromosomal endolysin NucD, encoded by a prophage remnant in Serratia marcescens, and the endolysin R from Qin, a cryptic prophage segment from E. coli K-12 (GI26249022), both of which have been demonstrated to have lytic function. Accession nos. refer to the GenBank database.

In one embodiment, at least one endolysin is from a bacteriophage. In certain embodiments, suitable endolysins may be from phages detailed in section 1(b) above in reference to the first protein. In another embodiment, at least one endolysin is from a Salmonella bacteriophage. In yet another embodiment, at least one endolysin is from a P22 phage. In still yet another embodiment, at least one endolysin is from a λ phage. In an alternative embodiment, at least one endolysin is gp19 from a P22 phage. In another alternative, a nucleic acid of the invention comprises gp19 and gp15 from a P22 phage. In some embodiments, at least one endolysin is R from a λ phage. In other embodiments, a nucleic acid of the invention comprises R and Rz from a λ phage. In certain embodiments, a nucleic acid of the invention comprises gp19, gp15, R, and Rz.

Additionally, an edolysin may be a protein described above with at least one, or a combination of one or more, nucleic acid deletions, substitutions, additions, or insertions which result in an alteration in the corresponding amino acid sequence of the encoded holin protein, such as a homolog, ortholog, mimic or degenerative variant. Such an endolysin may be generated using recombinant techniques such as those described in section I(b) above in reference to a first protein. In addition to having a substantially similar biological function, a homolog, ortholog, mimic or degenerative variant of an endolysin suitable for use in the invention will also typically share substantial sequence similarity to an endolysin protein. In addition, suitable homologs, orthologs, mimics or degenerative variants preferably share at least 30% sequence homology with an endolysin protein, more preferably, 50%, and even more preferably, are greater than about 75% homologous in sequence to an endolysin protein. Alternatively, peptide mimics of an endolysin could be used that retain critical molecular recognition elements, although peptide bonds, side chain structures, chiral centers and other features of the parental active protein sequence may be replaced by chemical entities that are not native to the endolysin protein yet, nevertheless, confer activity. Percent homology may be calculated as described in section 1(b) above.

### (d) Additional Components

In certain embodiments, nucleic acids of the invention may further comprise additional components, such as a marker, a spacer domain, and a flanking sequence.

i. Markers

In one embodiment, a nucleic acid of the invention comprises at least one marker. Generally speaking, a marker encodes a product that the host cell cannot make, such that the cell acquires resistance to a specific compound, is able to survive under specific conditions, or is otherwise differentiable from cells that do not carry the marker. Markers may be positive or negative markers. In some embodiments, a nucleic acid of the invention may comprise both a positive marker and a negative marker. In certain embodiments, the marker may code for an antibiotic resistance factor. Suitable examples of antibiotic resistance markers may include, but are not limited to, those coding for proteins that impart resistance to kanamycin, spectromycin, neomycin, geneticin (G418), ampicillin, tetracycline, and chloramphenicol. Additionally, the sacB gene may be used as a negative marker. The sacB gene is lethal in many bacteria when they are grown on sucrose media. Additionally, fluorescent proteins may be used as visually identifiable markers. Generally speaking, markers may be present during construction of the strains, but are typically removed from the final constructs.

ii. Spacer Domain

Additionally, a nucleic acid of the invention may comprise a Shine-Dalgarno sequence, or a ribsome binding site (RBS). Generally speaking, a RBS is the nucleic acid sequence in the mRNA that binds to a 16s rRNA in the ribosome to initiate translation. For gram negative bacteria, the RBS is generally AGGA. The RBS may be located about 8 to about 11 bp 3′ of the start codon of the first structural gene. One skilled in the art will realize that the RBS sequence or its distance to the start codon may be altered to increase or decrease translation efficiency.

iii. Flanking Sequence

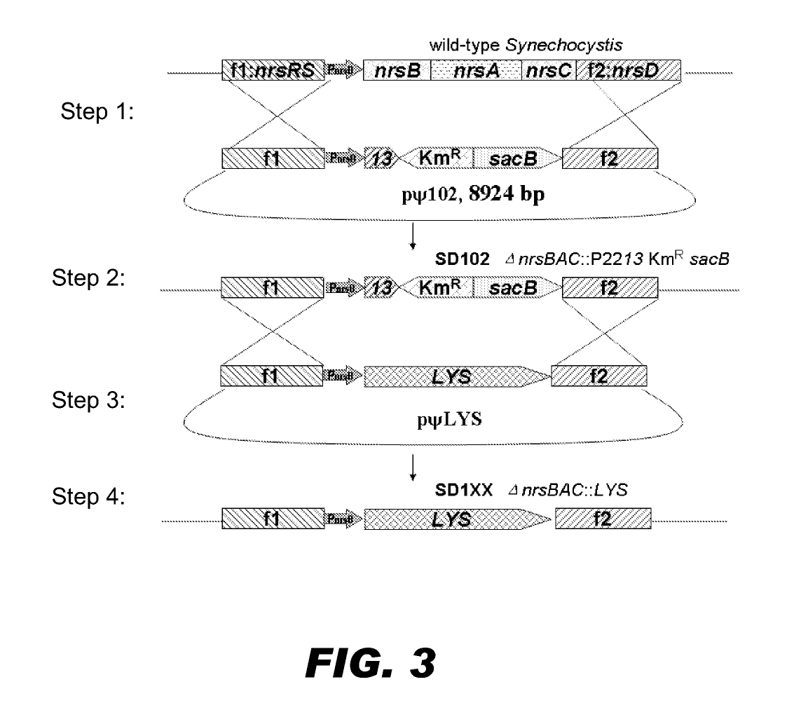

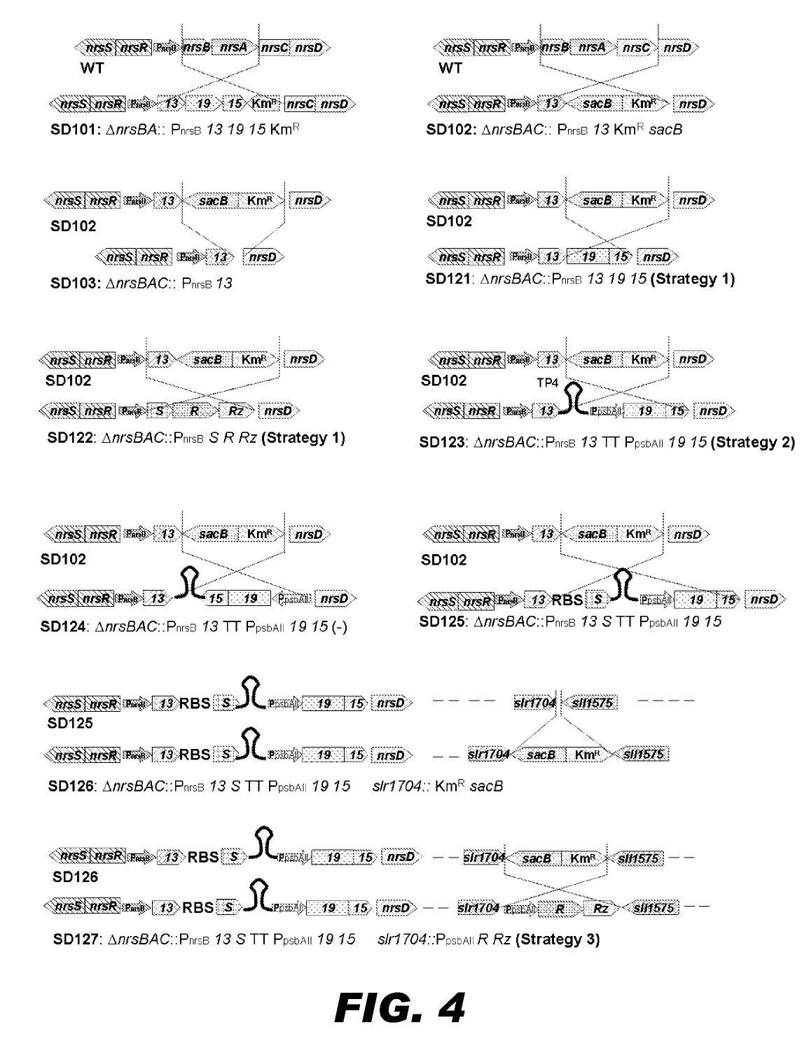

Nucleic acid constructs of the invention may also comprise flanking sequences. The phrase “flanking sequence” as used herein, refers to a nucleic acid sequence homologous to a chromosomal sequence. A construct comprising a flanking sequence on either side of a construct (i.e. a left flanking sequence and a right flanking sequence) may homologously recombine with the homologous chromosome, thereby integrating the construct between the flanking sequences into the chromosome. Generally speaking, flanking sequences may be of variable length. In an exemplary embodiment, the flanking sequences may be between about 300 and about 500 bp. In another exemplary embodiment, the left flanking sequence and the right flanking sequence are substantially the same length. For more details, see FIGS. 3 and 4, and the Examples.

### (e) Plasmids

A nucleic acid construct of the invention may comprise a plasmid suitable for use in a bacterium. Such a plasmid may contain multiple cloning sites for ease in manipulating nucleic acid sequences. Numerous suitable plasmids are known in the art.

Non-limiting examples of first inducible promoters, first proteins, second promoters, and endolysin combinations are listed in Table A below.

TABLE A

| First promoter | | Second | |
|---|---|---|---|
| induced by | First protein | promoter | Endolysin |
| Metal or metal ion | Cyanophage holin | — | At least one Cyanophage endolysin |
| Metal or metal ion | Cyanophage holin | — | At least one λ phage endolysin |
| Metal or metal ion | Cyanophage holin | — | P22 gene 19 |
| Metal or metal ion | Cyanophage holin | — | P22 gene 15 |
| Metal or metal ion | Cyanophage holin | — | P22 gene 19 and P22 gene 15 |
| Metal or metal ion | Cyanophage holin | — | At least one λ phage endolysin and at least one P22 phage |
| | | | endolysin |
| Metal or metal ion | P22 gene13 | — | At least one Cyanophage endolysin |
| Metal or metal ion | P22 gene13 | — | At least one λ phage endolysin |
| Metal or metal ion | P22 gene13 | — | P22 gene 19 |
| Metal or metal ion | P22 gene13 | — | P22 gene 15 |
| Metal or metal ion | P22 gene13 | — | P22 gene 19 and P22 gene 15 |
| Metal or metal ion | P22 gene13 | — | At least one λ phage endolysin and at least one P22 phage |
| | | | endolysin |
| Metal or metal ion | λ phage holin | — | At least one Cyanophage endolysin |
| Metal or metal ion | λ phage holin | — | At least one λ phage endolysin |
| Metal or metal ion | λ phage holin | — | P22 gene 19 |
| Metal or metal ion | λ phage holin | — | P22 gene 15 |
| Metal or metal ion | λ phage holin | — | P22 gene 19 and P22 gene 15 |
| Metal or metal ion | λ phage holin | — | At least one λ phage endolysin and at least one P22 phage |
| | | | endolysin |
| Metal or metal ion | A λ phage holin and a P22 | — | At least one Cyanophage endolysin |
| | phage holin | | |
| Metal or metal ion | λ phage holin and a P22 | — | At least one λ phage endolysin |
| | phage holin | | |
| Metal or metal ion | λ phage holin and a P22 | — | P22 gene 19 |
| | phage holin | | |
| Metal or metal ion | A λ phage holin and a P22 | — | P22 gene 15 |
| | phage holin | | |
| Metal or metal ion | A λ phage holin and a P22 | — | P22 gene 19 and P22 gene 15 |
| | phage holin | | |
| Metal or metal ion | A λ phage holin and a P22 | — | At least one λ phage endolysin and at least one P22 phage |
| | phage holin | | endolysin |
| Nickel or nickel ion | Cyanophage holin | — | At least one Cyanophage endolysin |
| Nickel or nickel ion | Cyanophage holin | — | At least one λ phage endolysin |
| Nickel or nickel ion | Cyanophage holin | — | P22 gene 19 |
| Nickel or nickel ion | Cyanophage holin | — | P22 gene 15 |
| Nickel or nickel ion | Cyanophage holin | — | P22 gene 19 and P22 gene 15 |
| Nickel or nickel ion | Cyanophage holin | — | At least one λ phage endolysin and at least one P22 phage |
| | | | endolysin |
| Nickel or nickel ion | P22 gene13 | — | At least one Cyanophage endolysin |
| Nickel or nickel ion | P22 gene13 | — | At least one λ phage endolysin |
| Nickel or nickel ion | P22 gene13 | — | P22 gene 19 |
| Nickel or nickel ion | P22 gene13 | — | P22 gene 15 |
| Nickel or nickel ion | P22 gene13 | — | P22 gene 19 and P22 gene 15 |
| Nickel or nickel ion | P22 gene13 | — | At least one λ phage endolysin and at least one P22 phage |
| | | | endolysin |
| Nickel or nickel ion | λ phage holin | — | At least one Cyanophage endolysin |
| Nickel or nickel ion | λ phage holin | — | At least one λ phage endolysin |
| Nickel or nickel ion | λ phage holin | — | P22 gene 19 |
| Nickel or nickel ion | λ phage holin | — | P22 gene 15 |
| Nickel or nickel ion | λ phage holin | — | P22 gene 19 and P22 gene 15 |
| Nickel or nickel ion | λ phage holin | — | At least one λ phage endolysin and at least one P22 phage |
| | | | endolysin |
| Nickel or nickel ion | A λ phage holin and a P22 | — | At least one Cyanophage endolysin |
| | phage holin | | |
| Nickel or nickel ion | λ phage holin and a P22 | — | At least one λ phage endolysin |
| | phage holin | | |
| Nickel or nickel ion | λ phage holin and a P22 | — | P22 gene 19 |
| | phage holin | | |
| Nickel or nickel ion | A λ phage holin and a P22 | — | P22 gene 15 |
| | phage holin | | |
| Nickel or nickel ion | A λ phage holin and a P22 | — | P22 gene 19 and P22 gene 15 |
| | phage holin | | |
| Nickel or nickel ion | A λ phage holin and a P22 | — | At least one λ phage endolysin and at least one P22 phage |
| | phage holin | | endolysin |
| Zinc or zinc ion | λ phage holin | — | At least one Cyanophage endolysin |
| Zinc or zinc ion | λ phage holin | — | At least one λ phage endolysin |
| Zinc or zinc ion | λ phage holin | — | P22 gene 19 |
| Zinc or zinc ion | λ phage holin | — | P22 gene 15 |
| Zinc or zinc ion | λ phage holin | — | P22 gene 19 and P22 gene 15 |
| Zinc or zinc ion | λ phage holin | — | At least one λ phage endolysin and at least one P22 phage |
| | | | endolysin |
| Zinc or zinc ion | Cyanophage holin | — | At least one Cyanophage endolysin |
| Zinc or zinc ion | Cyanophage holin | — | At least one λ phage endolysin |
| Zinc or zinc ion | Cyanophage holin | — | P22 gene 19 |
| Zinc or zinc ion | Cyanophage holin | — | P22 gene 15 |
| Zinc or zinc ion | Cyanophage holin | — | P22 gene 19 and P22 gene 15 |
| Zinc or zinc ion | Cyanophage holin | — | At least one λ phage endolysin and at least one P22 phage |
| | | | endolysin |
| Zinc or zinc ion | P22 gene13 | — | At least one Cyanophage endolysin |
| Zinc or zinc ion | P22 gene13 | — | At least one λ phage endolysin |
| Zinc or zinc ion | P22 gene13 | — | P22 gene 19 |
| Zinc or zinc ion | P22 gene13 | — | P22 gene 15 |
| Zinc or zinc ion | P22 gene13 | — | P22 gene 19 and P22 gene 15 |
| Zinc or zinc ion | P22 gene13 | — | At least one λ phage endolysin and at least one P22 phage |
| | | | endolysin |
| Zinc or zinc ion | A λ phage holin and a P22 | — | At least one Cyanophage endolysin |
| | phage holin | | |
| Zinc or zinc ion | λ phage holin and a P22 | — | At least one λ phage endolysin |
| | phage holin | | |
| Zinc or zinc ion | λ phage holin and a P22 | — | P22 gene 19 |
| | phage holin | | |
| Zinc or zinc ion | A λ phage holin and a P22 | — | P22 gene 15 |
| | phage holin | | |
| Zinc or zinc ion | A λ phage holin and a P22 | — | P22 gene 19 and P22 gene 15 |
| | phage holin | | |
| Zinc or zinc ion | A λ phage holin and a P22 | — | At least one λ phage endolysin and at least one P22 phage |
| | phage holin | | endolysin |
| Copper or copper ion | λ phage holin | — | At least one Cyanophage endolysin |
| Copper or copper ion | λ phage holin | — | At least one λ phage endolysin |
| Copper or copper ion | λ phage holin | — | P22 gene 19 |
| Copper or copper ion | λ phage holin | — | P22 gene 15 |
| Copper or copper ion | λ phage holin | — | P22 gene 19 and P22 gene 15 |
| Copper or copper ion | λ phage holin | — | At least one λ phage endolysin and at least one P22 phage |
| | | | endolysin |
| Copper or copper ion | Cyanophage holin | — | At least one Cyanophage endolysin |
| Copper or copper ion | Cyanophage holin | — | At least one λ phage endolysin |
| Copper or copper ion | Cyanophage holin | — | P22 gene 19 |
| Copper or copper ion | Cyanophage holin | — | P22 gene 15 |
| Copper or copper ion | Cyanophage holin | — | P22 gene 19 and P22 gene 15 |
| Copper or copper ion | Cyanophage holin | — | At least one λ phage endolysin and at least one P22 phage |
| | | | endolysin |
| Copper or copper ion | P22 gene13 | — | At least one Cyanophage endolysin |
| Copper or copper ion | P22 gene13 | — | At least one λ phage endolysin |
| Copper or copper ion | P22 gene13 | — | P22 gene 19 |
| Copper or copper ion | P22 gene13 | — | P22 gene 15 |
| Copper or copper ion | P22 gene13 | — | P22 gene 19 and P22 gene 15 |
| Copper or copper ion | P22 gene13 | — | At least one λ phage endolysin and at least one P22 phage |
| | | | endolysin |
| Copper or copper ion | A λ phage holin and a P22 | — | At least one Cyanophage endolysin |
| | phage holin | | |
| Copper or copper ion | λ phage holin and a P22 | — | At least one λ phage endolysin |
| | phage holin | | |
| Copper or copper ion | λ phage holin and a P22 | — | P22 gene 19 |
| | phage holin | | |
| Copper or copper ion | A λ phage holin and a P22 | — | P22 gene 15 |
| | phage holin | | |
| Copper or copper ion | A λ phage holin and a P22 | — | P22 gene 19 and P22 gene 15 |
| | phage holin | | |
| Copper or copper ion | A λ phage holin and a P22 | — | At least one λ phage endolysin and at least one P22 phage |
| | phage holin | | endolysin |
| Gold or gold ion | λ phage holin | — | At least one Cyanophage endolysin |
| Gold or gold ion | λ phage holin | — | At least one λ phage endolysin |
| Gold or gold ion | λ phage holin | — | P22 gene 19 |
| Gold or gold ion | λ phage holin | — | P22 gene 15 |
| Gold or gold ion | λ phage holin | — | P22 gene 19 and P22 gene 15 |
| Gold or gold ion | λ phage holin | — | At least one λ phage endolysin and at least one P22 phage |
| | | | endolysin |
| Gold or gold ion | Cyanophage holin | — | At least one Cyanophage endolysin |
| Gold or gold ion | Cyanophage holin | — | At least one λ phage endolysin |
| Gold or gold ion | Cyanophage holin | — | P22 gene 19 |
| Gold or gold ion | Cyanophage holin | — | P22 gene 15 |
| Gold or gold ion | Cyanophage holin | — | P22 gene 19 and P22 gene 15 |
| Gold or gold ion | Cyanophage holin | — | At least one λ phage endolysin and at least one P22 phage |
| | | | endolysin |
| Gold or gold ion | P22 gene13 | — | At least one Cyanophage endolysin |
| Gold or gold ion | P22 gene13 | — | At least one λ phage endolysin |
| Gold or gold ion | P22 gene13 | — | P22 gene 19 |
| Gold or gold ion | P22 gene13 | — | P22 gene 15 |
| Gold or gold ion | P22 gene13 | — | P22 gene 19 and P22 gene 15 |
| Gold or gold ion | P22 gene13 | — | At least one λ phage endolysin and at least one P22 phage |
| | | | endolysin |
| Gold or gold ion | A λ phage holin and a P22 | — | At least one Cyanophage endolysin |
| | phage holin | | |
| Gold or gold ion | λ phage holin and a P22 | — | At least one λ phage endolysin |
| | phage holin | | |
| Gold or gold ion | λ phage holin and a P22 | — | P22 gene 19 |
| | phage holin | | |
| Gold or gold ion | A λ phage holin and a P22 | — | P22 gene 15 |
| | phage holin | | |
| Gold or gold ion | A λ phage holin and a P22 | — | P22 gene 19 and P22 gene 15 |
| | phage holin | | |
| Gold or gold ion | A λ phage holin and a P22 | — | At least one λ phage endolysin and at least one P22 phage |
| | phage holin | | endolysin |
| Silver or silver ion | λ phage holin | — | At least one Cyanophage endolysin |
| Silver or silver ion | λ phage holin | — | At least one λ phage endolysin |
| Silver or silver ion | λ phage holin | — | P22 gene 19 |
| Silver or silver ion | λ phage holin | — | P22 gene 15 |
| Silver or silver ion | λ phage holin | — | P22 gene 19 and P22 gene 15 |
| Silver or silver ion | λ phage holin | — | At least one λ phage endolysin and at least one P22 phage |
| | | | endolysin |
| Silver or silver ion | Cyanophage holin | — | At least one Cyanophage endolysin |
| Silver or silver ion | Cyanophage holin | — | At least one λ phage endolysin |
| Silver or silver ion | Cyanophage holin | — | P22 gene 19 |
| Silver or silver ion | Cyanophage holin | — | P22 gene 15 |
| Silver or silver ion | Cyanophage holin | — | P22 gene 19 and P22 gene 15 |
| Silver or silver ion | Cyanophage holin | — | At least one λ phage endolysin and at least one P22 phage |
| | | | endolysin |
| Silver or silver ion | P22 gene13 | — | At least one Cyanophage endolysin |
| Silver or silver ion | P22 gene13 | — | At least one λ phage endolysin |
| Silver or silver ion | P22 gene13 | — | P22 gene 19 |
| Silver or silver ion | P22 gene13 | — | P22 gene 15 |
| Silver or silver ion | P22 gene13 | — | P22 gene 19 and P22 gene 15 |
| Silver or silver ion | P22 gene13 | — | At least one λ phage endolysin and at least one P22 phage |
| | | | endolysin |
| Silver or silver ion | A λ phage holin and a P22 | — | At least one Cyanophage endolysin |
| | phage holin | | |
| Silver or silver ion | λ phage holin and a P22 | — | At least one λ phage endolysin |
| | phage holin | | |
| Silver or silver ion | λ phage holin and a P22 | — | P22 gene 19 |
| | phage holin | | |
| Silver or silver ion | A λ phage holin and a P22 | — | P22 gene 15 |
| | phage holin | | |
| Silver or silver ion | A λ phage holin and a P22 | — | P22 gene 19 and P22 gene 15 |
| | phage holin | | |
| Silver or silver ion | A λ phage holin and a P22 | — | At least one λ phage endolysin and at least one P22 phage |
| | phage holin | | endolysin |
| Metal or metal ion | Cyanophage holin | constitutive | At least one Cyanophage endolysin |
| Metal or metal ion | Cyanophage holin | constitutive | At least one λ phage endolysin |
| Metal or metal ion | Cyanophage holin | constitutive | P22 gene 19 |
| Metal or metal ion | Cyanophage holin | constitutive | P22 gene 15 |
| Metal or metal ion | Cyanophage holin | constitutive | P22 gene 19 and P22 gene 15 |
| Metal or metal ion | Cyanophage holin | constitutive | At least one λ phage endolysin and at least one P22 phage |
| | | | endolysin |
| Metal or metal ion | P22 gene13 | constitutive | At least one Cyanophage endolysin |
| Metal or metal ion | P22 gene13 | constitutive | At least one λ phage endolysin |
| Metal or metal ion | P22 gene13 | constitutive | P22 gene 19 |
| Metal or metal ion | P22 gene13 | constitutive | P22 gene 15 |
| Metal or metal ion | P22 gene13 | constitutive | P22 gene 19 and P22 gene 15 |
| Metal or metal ion | P22 gene13 | constitutive | At least one λ phage endolysin and at least one P22 phage |
| | | | endolysin |
| Metal or metal ion | λ phage holin | constitutive | At least one Cyanophage endolysin |
| Metal or metal ion | λ phage holin | constitutive | At least one λ phage endolysin |
| Metal or metal ion | λ phage holin | constitutive | P22 gene 19 |
| Metal or metal ion | λ phage holin | constitutive | P22 gene 15 |
| Metal or metal ion | λ phage holin | constitutive | P22 gene 19 and P22 gene 15 |
| Metal or metal ion | λ phage holin | constitutive | At least one λ phage endolysin and at least one P22 phage |
| | | | endolysin |
| Metal or metal ion | A λ phage holin and a P22 | constitutive | At least one Cyanophage endolysin |
| | phage holin | | |
| Metal or metal ion | λ phage holin and a P22 | constitutive | At least one λ phage endolysin |
| | phage holin | | |
| Metal or metal ion | λ phage holin and a P22 | constitutive | P22 gene 19 |
| | phage holin | | |
| Metal or metal ion | A λ phage holin and a P22 | constitutive | P22 gene 15 |
| | phage holin | | |
| Metal or metal ion | A λ phage holin and a P22 | constitutive | P22 gene 19 and P22 gene 15 |
| | phage holin | | |
| Metal or metal ion | A λ phage holin and a P22 | constitutive | At least one λ phage endolysin and at least one P22 phage |
| | phage holin | | endolysin |
| Nickel or nickel ion | Cyanophage holin | constitutive | At least one Cyanophage endolysin |
| Nickel or nickel ion | Cyanophage holin | constitutive | At least one λ phage endolysin |
| Nickel or nickel ion | Cyanophage holin | constitutive | P22 gene 19 |
| Nickel or nickel ion | Cyanophage holin | constitutive | P22 gene 15 |
| Nickel or nickel ion | Cyanophage holin | constitutive | P22 gene 19 and P22 gene 15 |
| Nickel or nickel ion | Cyanophage holin | constitutive | At least one λ phage endolysin and at least one P22 phage |
| | | | endolysin |
| Nickel or nickel ion | P22 gene13 | constitutive | At least one Cyanophage endolysin |
| Nickel or nickel ion | P22 gene13 | constitutive | At least one λ phage endolysin |
| Nickel or nickel ion | P22 gene13 | constitutive | P22 gene 19 |
| Nickel or nickel ion | P22 gene13 | constitutive | P22 gene 15 |
| Nickel or nickel ion | P22 gene13 | constitutive | P22 gene 19 and P22 gene 15 |
| Nickel or nickel ion | P22 gene13 | constitutive | At least one λ phage endolysin and at least one P22 phage |
| | | | endolysin |
| Nickel or nickel ion | λ phage holin | constitutive | At least one Cyanophage endolysin |
| Nickel or nickel ion | λ phage holin | constitutive | At least one λ phage endolysin |
| Nickel or nickel ion | λ phage holin | constitutive | P22 gene 19 |
| Nickel or nickel ion | λ phage holin | constitutive | P22 gene 15 |
| Nickel or nickel ion | λ phage holin | constitutive | P22 gene 19 and P22 gene 15 |
| Nickel or nickel ion | λ phage holin | constitutive | At least one λ phage endolysin and at least one P22 phage |
| | | | endolysin |
| Nickel or nickel ion | A λ phage holin and a P22 | constitutive | At least one Cyanophage endolysin |
| | phage holin | | |
| Nickel or nickel ion | λ phage holin and a P22 | constitutive | At least one λ phage endolysin |
| | phage holin | | |
| Nickel or nickel ion | λ phage holin and a P22 | constitutive | P22 gene 19 |
| | phage holin | | |
| Nickel or nickel ion | A λ phage holin and a P22 | constitutive | P22 gene 15 |
| | phage holin | | |
| Nickel or nickel ion | A λ phage holin and a P22 | constitutive | P22 gene 19 and P22 gene 15 |
| | phage holin | | |
| Nickel or nickel ion | A λ phage holin and a P22 | constitutive | At least one λ phage endolysin and at least one P22 phage |
| | phage holin | | endolysin |
| Zinc or zinc ion | λ phage holin | constitutive | At least one Cyanophage endolysin |
| Zinc or zinc ion | λ phage holin | constitutive | At least one λ phage endolysin |
| Zinc or zinc ion | λ phage holin | constitutive | P22 gene 19 |
| Zinc or zinc ion | λ phage holin | constitutive | P22 gene 15 |
| Zinc or zinc ion | λ phage holin | constitutive | P22 gene 19 and P22 gene 15 |
| Zinc or zinc ion | λ phage holin | constitutive | At least one λ phage endolysin and at least one P22 phage |
| | | | endolysin |
| Zinc or zinc ion | Cyanophage holin | constitutive | At least one Cyanophage endolysin |
| Zinc or zinc ion | Cyanophage holin | constitutive | At least one λ phage endolysin |
| Zinc or zinc ion | Cyanophage holin | constitutive | P22 gene 19 |
| Zinc or zinc ion | Cyanophage holin | constitutive | P22 gene 15 |
| Zinc or zinc ion | Cyanophage holin | constitutive | P22 gene 19 and P22 gene 15 |
| Zinc or zinc ion | Cyanophage holin | constitutive | At least one λ phage endolysin and at least one P22 phage |
| | | | endolysin |
| Zinc or zinc ion | P22 gene13 | constitutive | At least one Cyanophage endolysin |
| Zinc or zinc ion | P22 gene13 | constitutive | At least one λ phage endolysin |
| Zinc or zinc ion | P22 gene13 | constitutive | P22 gene 19 |
| Zinc or zinc ion | P22 gene13 | constitutive | P22 gene 15 |
| Zinc or zinc ion | P22 gene13 | constitutive | P22 gene 19 and P22 gene 15 |
| Zinc or zinc ion | P22 gene13 | constitutive | At least one λ phage endolysin and at least one P22 phage |
| | | | endolysin |
| Zinc or zinc ion | A λ phage holin and a P22 | constitutive | At least one Cyanophage endolysin |
| | phage holin | | |
| Zinc or zinc ion | λ phage holin and a P22 | constitutive | At least one λ phage endolysin |
| | phage holin | | |
| Zinc or zinc ion | λ phage holin and a P22 | constitutive | P22 gene 19 |
| | phage holin | | |
| Zinc or zinc ion | A λ phage holin and a P22 | constitutive | P22 gene 15 |
| | phage holin | | |
| Zinc or zinc ion | A λ phage holin and a P22 | constitutive | P22 gene 19 and P22 gene 15 |
| | phage holin | | |
| Zinc or zinc ion | A λ phage holin and a P22 | constitutive | At least one λ phage endolysin and at least one P22 phage |
| | phage holin | | endolysin |
| Copper or copper ion | λ phage holin | constitutive | At least one Cyanophage endolysin |
| Copper or copper ion | λ phage holin | constitutive | At least one λ phage endolysin |
| Copper or copper ion | λ phage holin | constitutive | P22 gene 19 |
| Copper or copper ion | λ phage holin | constitutive | P22 gene 15 |
| Copper or copper ion | λ phage holin | constitutive | P22 gene 19 and P22 gene 15 |
| Copper or copper ion | λ phage holin | constitutive | At least one λ phage endolysin and at least one P22 phage |
| | | | endolysin |
| Copper or copper ion | Cyanophage holin | constitutive | At least one Cyanophage endolysin |
| Copper or copper ion | Cyanophage holin | constitutive | At least one λ phage endolysin |
| Copper or copper ion | Cyanophage holin | constitutive | P22 gene 19 |
| Copper or copper ion | Cyanophage holin | constitutive | P22 gene 15 |
| Copper or copper ion | Cyanophage holin | constitutive | P22 gene 19 and P22 gene 15 |
| Copper or copper ion | Cyanophage holin | constitutive | At least one λ phage endolysin and at least one P22 phage |
| | | | endolysin |
| Copper or copper ion | P22 gene13 | constitutive | At least one Cyanophage endolysin |
| Copper or copper ion | P22 gene13 | constitutive | At least one λ phage endolysin |
| Copper or copper ion | P22 gene13 | constitutive | P22 gene 19 |
| Copper or copper ion | P22 gene13 | constitutive | P22 gene 15 |
| Copper or copper ion | P22 gene13 | constitutive | P22 gene 19 and P22 gene 15 |
| Copper or copper ion | P22 gene13 | constitutive | At least one λ phage endolysin and at least one P22 phage |
| | | | endolysin |
| Copper or copper ion | A λ phage holin and a P22 | constitutive | At least one Cyanophage endolysin |
| | phage holin | | |
| Copper or copper ion | λ phage holin and a P22 | constitutive | At least one λ phage endolysin |
| | phage holin | | |
| Copper or copper ion | λ phage holin and a P22 | constitutive | P22 gene 19 |
| | phage holin | | |
| Copper or copper ion | A λ phage holin and a P22 | constitutive | P22 gene 15 |
| | phage holin | | |
| Copper or copper ion | A λ phage holin and a P22 | constitutive | P22 gene 19 and P22 gene 15 |
| | phage holin | | |
| Copper or copper ion | A λ phage holin and a P22 | constitutive | At least one λ phage endolysin and at least one P22 phage |
| | phage holin | | endolysin |
| Gold or gold ion | λ phage holin | constitutive | At least one Cyanophage endolysin |
| Gold or gold ion | λ phage holin | constitutive | At least one λ phage endolysin |
| Gold or gold ion | λ phage holin | constitutive | P22 gene 19 |
| Gold or gold ion | λ phage holin | constitutive | P22 gene 15 |
| Gold or gold ion | λ phage holin | constitutive | P22 gene 19 and P22 gene 15 |
| Gold or gold ion | λ phage holin | constitutive | At least one λ phage endolysin and at least one P22 phage |
| | | | endolysin |
| Gold or gold ion | Cyanophage holin | constitutive | At least one Cyanophage endolysin |
| Gold or gold ion | Cyanophage holin | constitutive | At least one λ phage endolysin |
| Gold or gold ion | Cyanophage holin | constitutive | P22 gene 19 |
| Gold or gold ion | Cyanophage holin | constitutive | P22 gene 15 |
| Gold or gold ion | Cyanophage holin | constitutive | P22 gene 19 and P22 gene 15 |
| Gold or gold ion | Cyanophage holin | constitutive | At least one λ phage endolysin and at least one P22 phage |
| | | | endolysin |
| Gold or gold ion | P22 gene13 | constitutive | At least one Cyanophage endolysin |
| Gold or gold ion | P22 gene13 | constitutive | At least one λ phage endolysin |
| Gold or gold ion | P22 gene13 | constitutive | P22 gene 19 |
| Gold or gold ion | P22 gene13 | constitutive | P22 gene 15 |
| Gold or gold ion | P22 gene13 | constitutive | P22 gene 19 and P22 gene 15 |
| Gold or gold ion | P22 gene13 | constitutive | At least one λ phage endolysin and at least one P22 phage |
| | | | endolysin |
| Gold or gold ion | A λ phage holin and a P22 | constitutive | At least one Cyanophage endolysin |
| | phage holin | | |
| Gold or gold ion | λ phage holin and a P22 | constitutive | At least one λ phage endolysin |
| | phage holin | | |
| Gold or gold ion | λ phage holin and a P22 | constitutive | P22 gene 19 |
| | phage holin | | |
| Gold or gold ion | A λ phage holin and a P22 | constitutive | P22 gene 15 |
| | phage holin | | |
| Gold or gold ion | A λ phage holin and a P22 | constitutive | P22 gene 19 and P22 gene 15 |
| | phage holin | | |
| Gold or gold ion | A λ phage holin and a P22 | constitutive | At least one λ phage endolysin and at least one P22 phage |
| | phage holin | | endolysin |
| Silver or silver ion | λ phage holin | constitutive | At least one Cyanophage endolysin |
| Silver or silver ion | λ phage holin | constitutive | At least one λ phage endolysin |
| Silver or silver ion | λ phage holin | constitutive | P22 gene 19 |
| Silver or silver ion | λ phage holin | constitutive | P22 gene 15 |
| Silver or silver ion | λ phage holin | constitutive | P22 gene 19 and P22 gene 15 |
| Silver or silver ion | λ phage holin | constitutive | At least one λ phage endolysin and at least one P22 phage |
| | | | endolysin |
| Silver or silver ion | Cyanophage holin | constitutive | At least one Cyanophage endolysin |
| Silver or silver ion | Cyanophage holin | constitutive | At least one λ phage endolysin |
| Silver or silver ion | Cyanophage holin | constitutive | P22 gene 19 |
| Silver or silver ion | Cyanophage holin | constitutive | P22 gene 15 |
| Silver or silver ion | Cyanophage holin | constitutive | P22 gene 19 and P22 gene 15 |
| Silver or silver ion | Cyanophage holin | constitutive | At least one λ phage endolysin and at least one P22 phage |
| | | | endolysin |
| Silver or silver ion | P22 gene13 | constitutive | At least one Cyanophage endolysin |
| Silver or silver ion | P22 gene13 | constitutive | At least one λ phage endolysin |
| Silver or silver ion | P22 gene13 | constitutive | P22 gene 19 |
| Silver or silver ion | P22 gene13 | constitutive | P22 gene 15 |
| Silver or silver ion | P22 gene13 | constitutive | P22 gene 19 and P22 gene 15 |
| Silver or silver ion | P22 gene13 | constitutive | At least one λ phage endolysin and at least one P22 phage |
| | | | endolysin |
| Silver or silver ion | A λ phage holin and a P22 | constitutive | At least one Cyanophage endolysin |
| | phage holin | | |
| Silver or silver ion | λ phage holin and a P22 | constitutive | At least one λ phage endolysin |
| | phage holin | | |
| Silver or silver ion | λ phage holin and a P22 | constitutive | P22 gene 19 |
| | phage holin | | |
| Silver or silver ion | A λ phage holin and a P22 | constitutive | P22 gene 15 |
| | phage holin | | |
| Silver or silver ion | A λ phage holin and a P22 | constitutive | P22 gene 19 and P22 gene 15 |
| | phage holin | | |
| Silver or silver ion | A λ phage holin and a P22 | constitutive | At least one λ phage endolysin and at least one P22 phage |
| | phage holin | | endolysin |
| Metal or metal ion | Cyanophage holin | inducible | At least one Cyanophage endolysin |
| Metal or metal ion | Cyanophage holin | inducible | At least one λ phage endolysin |
| Metal or metal ion | Cyanophage holin | inducible | P22 gene 19 |
| Metal or metal ion | Cyanophage holin | inducible | P22 gene 15 |
| Metal or metal ion | Cyanophage holin | inducible | P22 gene 19 and P22 gene 15 |
| Metal or metal ion | Cyanophage holin | inducible | At least one λ phage endolysin and at least one P22 phage |
| | | | endolysin |
| Metal or metal ion | P22 gene13 | inducible | At least one Cyanophage endolysin |
| Metal or metal ion | P22 gene13 | inducible | At least one λ phage endolysin |
| Metal or metal ion | P22 gene13 | inducible | P22 gene 19 |
| Metal or metal ion | P22 gene13 | inducible | P22 gene 15 |
| Metal or metal ion | P22 gene13 | inducible | P22 gene 19 and P22 gene 15 |
| Metal or metal ion | P22 gene13 | inducible | At least one λ phage endolysin and at least one P22 phage |
| | | | endolysin |
| Metal or metal ion | λ phage holin | inducible | At least one Cyanophage endolysin |
| Metal or metal ion | λ phage holin | inducible | At least one λ phage endolysin |
| Metal or metal ion | λ phage holin | inducible | P22 gene 19 |
| Metal or metal ion | λ phage holin | inducible | P22 gene 15 |
| Metal or metal ion | λ phage holin | inducible | P22 gene 19 and P22 gene 15 |
| Metal or metal ion | λ phage holin | inducible | At least one λ phage endolysin and at least one P22 phage |
| | | | endolysin |
| Metal or metal ion | A λ phage holin and a P22 | inducible | At least one Cyanophage endolysin |
| | phage holin | | |
| Metal or metal ion | λ phage holin and a P22 | inducible | At least one λ phage endolysin |
| | phage holin | | |
| Metal or metal ion | λ phage holin and a P22 | inducible | P22 gene 19 |
| | phage holin | | |
| Metal or metal ion | A λ phage holin and a P22 | inducible | P22 gene 15 |
| | phage holin | | |
| Metal or metal ion | A λ phage holin and a P22 | inducible | P22 gene 19 and P22 gene 15 |
| | phage holin | | |
| Metal or metal ion | A λ phage holin and a P22 | inducible | At least one λ phage endolysin and at least one P22 phage |
| | phage holin | | endolysin |
| Nickel or nickel ion | Cyanophage holin | inducible | At least one Cyanophage endolysin |
| Nickel or nickel ion | Cyanophage holin | inducible | At least one λ phage endolysin |
| Nickel or nickel ion | Cyanophage holin | inducible | P22 gene 19 |
| Nickel or nickel ion | Cyanophage holin | inducible | P22 gene 15 |
| Nickel or nickel ion | Cyanophage holin | inducible | P22 gene 19 and P22 gene 15 |
| Nickel or nickel ion | Cyanophage holin | inducible | At least one λ phage endolysin and at least one P22 phage |
| | | | endolysin |
| Nickel or nickel ion | P22 gene13 | inducible | At least one Cyanophage endolysin |
| Nickel or nickel ion | P22 gene13 | inducible | At least one λ phage endolysin |
| Nickel or nickel ion | P22 gene13 | inducible | P22 gene 19 |
| Nickel or nickel ion | P22 gene13 | inducible | P22 gene 15 |
| Nickel or nickel ion | P22 gene13 | inducible | P22 gene 19 and P22 gene 15 |
| Nickel or nickel ion | P22 gene13 | inducible | At least one λ phage endolysin and at least one P22 phage |
| | | | endolysin |
| Nickel or nickel ion | λ phage holin | inducible | At least one Cyanophage endolysin |
| Nickel or nickel ion | λ phage holin | inducible | At least one λ phage endolysin |
| Nickel or nickel ion | λ phage holin | inducible | P22 gene 19 |
| Nickel or nickel ion | λ phage holin | inducible | P22 gene 15 |
| Nickel or nickel ion | λ phage holin | inducible | P22 gene 19 and P22 gene 15 |
| Nickel or nickel ion | λ phage holin | inducible | At least one λ phage endolysin and at least one P22 phage |
| | | | endolysin |
| Nickel or nickel ion | A λ phage holin and a P22 | inducible | At least one Cyanophage endolysin |
| | phage holin | | |
| Nickel or nickel ion | λ phage holin and a P22 | inducible | At least one λ phage endolysin |
| | phage holin | | |
| Nickel or nickel ion | λ phage holin and a P22 | inducible | P22 gene 19 |
| | phage holin | | |
| Nickel or nickel ion | A λ phage holin and a P22 | inducible | P22 gene 15 |
| | phage holin | | |
| Nickel or nickel ion | A λ phage holin and a P22 | inducible | P22 gene 19 and P22 gene 15 |
| | phage holin | | |
| Nickel or nickel ion | A λ phage holin and a P22 | inducible | At least one λ phage endolysin and at least one P22 phage |
| | phage holin | | endolysin |
| Zinc or zinc ion | λ phage holin | inducible | At least one Cyanophage endolysin |
| Zinc or zinc ion | λ phage holin | inducible | At least one λ phage endolysin |
| Zinc or zinc ion | λ phage holin | inducible | P22 gene 19 |
| Zinc or zinc ion | λ phage holin | inducible | P22 gene 15 |
| Zinc or zinc ion | λ phage holin | inducible | P22 gene 19 and P22 gene 15 |
| Zinc or zinc ion | λ phage holin | inducible | At least one λ phage endolysin and at least one P22 phage |
| | | | endolysin |
| Zinc or zinc ion | Cyanophage holin | inducible | At least one Cyanophage endolysin |
| Zinc or zinc ion | Cyanophage holin | inducible | At least one λ phage endolysin |
| Zinc or zinc ion | Cyanophage holin | inducible | P22 gene 19 |
| Zinc or zinc ion | Cyanophage holin | inducible | P22 gene 15 |
| Zinc or zinc ion | Cyanophage holin | inducible | P22 gene 19 and P22 gene 15 |
| Zinc or zinc ion | Cyanophage holin | inducible | At least one λ phage endolysin and at least one P22 phage |
| | | | endolysin |
| Zinc or zinc ion | P22 gene13 | inducible | At least one Cyanophage endolysin |
| Zinc or zinc ion | P22 gene13 | inducible | At least one λ phage endolysin |
| Zinc or zinc ion | P22 gene13 | inducible | P22 gene 19 |
| Zinc or zinc ion | P22 gene13 | inducible | P22 gene 15 |
| Zinc or zinc ion | P22 gene13 | inducible | P22 gene 19 and P22 gene 15 |
| Zinc or zinc ion | P22 gene13 | inducible | At least one λ phage endolysin and at least one P22 phage |
| | | | endolysin |
| Zinc or zinc ion | A λ phage holin and a P22 | inducible | At least one Cyanophage endolysin |
| | phage holin | | |
| Zinc or zinc ion | λ phage holin and a P22 | inducible | At least one λ phage endolysin |
| | phage holin | | |
| Zinc or zinc ion | λ phage holin and a P22 | inducible | P22 gene 19 |
| | phage holin | | |
| Zinc or zinc ion | A λ phage holin and a P22 | inducible | P22 gene 15 |
| | phage holin | | |
| Zinc or zinc ion | A λ phage holin and a P22 | inducible | P22 gene 19 and P22 gene 15 |
| | phage holin | | |
| Zinc or zinc ion | A λ phage holin and a P22 | inducible | At least one λ phage endolysin and at least one P22 phage |
| | phage holin | | endolysin |
| Copper or copper ion | λ phage holin | inducible | At least one Cyanophage endolysin |
| Copper or copper ion | λ phage holin | inducible | At least one λ phage endolysin |
| Copper or copper ion | λ phage holin | inducible | P22 gene 19 |
| Copper or copper ion | λ phage holin | inducible | P22 gene 15 |
| Copper or copper ion | λ phage holin | inducible | P22 gene 19 and P22 gene 15 |
| Copper or copper ion | λ phage holin | inducible | At least one λ phage endolysin and at least one P22 phage |
| | | | endolysin |
| Copper or copper ion | Cyanophage holin | inducible | At least one Cyanophage endolysin |
| Copper or copper ion | Cyanophage holin | inducible | At least one λ phage endolysin |
| Copper or copper ion | Cyanophage holin | inducible | P22 gene 19 |
| Copper or copper ion | Cyanophage holin | inducible | P22 gene 15 |
| Copper or copper ion | Cyanophage holin | inducible | P22 gene 19 and P22 gene 15 |
| Copper or copper ion | Cyanophage holin | inducible | At least one λ phage endolysin and at least one P22 phage |
| | | | endolysin |
| Copper or copper ion | P22 gene13 | inducible | At least one Cyanophage endolysin |
| Copper or copper ion | P22 gene13 | inducible | At least one λ phage endolysin |
| Copper or copper ion | P22 gene13 | inducible | P22 gene 19 |
| Copper or copper ion | P22 gene13 | inducible | P22 gene 15 |
| Copper or copper ion | P22 gene13 | inducible | P22 gene 19 and P22 gene 15 |
| Copper or copper ion | P22 gene13 | inducible | At least one λ phage endolysin and at least one P22 phage |
| | | | endolysin |
| Copper or copper ion | A λ phage holin and a P22 | inducible | At least one Cyanophage endolysin |
| | phage holin | | |
| Copper or copper ion | λ phage holin and a P22 | inducible | At least one λ phage endolysin |
| | phage holin | | |
| Copper or copper ion | λ phage holin and a P22 | inducible | P22 gene 19 |
| | phage holin | | |
| Copper or copper ion | A λ phage holin and a P22 | inducible | P22 gene 15 |
| | phage holin | | |
| Copper or copper ion | A λ phage holin and a P22 | inducible | P22 gene 19 and P22 gene 15 |
| | phage holin | | |
| Copper or copper ion | A λ phage holin and a P22 | inducible | At least one λ phage endolysin and at least one P22 phage |
| | phage holin | | endolysin |
| Gold or gold ion | λ phage holin | inducible | At least one Cyanophage endolysin |
| Gold or gold ion | λ phage holin | inducible | At least one λ phage endolysin |
| Gold or gold ion | λ phage holin | inducible | P22 gene 19 |
| Gold or gold ion | λ phage holin | inducible | P22 gene 15 |
| Gold or gold ion | λ phage holin | inducible | P22 gene 19 and P22 gene 15 |
| Gold or gold ion | λ phage holin | inducible | At least one λ phage endolysin and at least one P22 phage |
| | | | endolysin |
| Gold or gold ion | Cyanophage holin | inducible | At least one Cyanophage endolysin |
| Gold or gold ion | Cyanophage holin | inducible | At least one λ phage endolysin |
| Gold or gold ion | Cyanophage holin | inducible | P22 gene 19 |
| Gold or gold ion | Cyanophage holin | inducible | P22 gene 15 |
| Gold or gold ion | Cyanophage holin | inducible | P22 gene 19 and P22 gene 15 |
| Gold or gold ion | Cyanophage holin | inducible | At least one λ phage endolysin and at least one P22 phage |
| | | | endolysin |
| Gold or gold ion | P22 gene13 | inducible | At least one Cyanophage endolysin |
| Gold or gold ion | P22 gene13 | inducible | At least one λ phage endolysin |
| Gold or gold ion | P22 gene13 | inducible | P22 gene 19 |
| Gold or gold ion | P22 gene13 | inducible | P22 gene 15 |
| Gold or gold ion | P22 gene13 | inducible | P22 gene 19 and P22 gene 15 |
| Gold or gold ion | P22 gene13 | inducible | At least one λ phage endolysin and at least one P22 phage |
| | | | endolysin |
| Gold or gold ion | A λ phage holin and a P22 | inducible | At least one Cyanophage endolysin |
| | phage holin | | |
| Gold or gold ion | λ phage holin and a P22 | inducible | At least one λ phage endolysin |
| | phage holin | | |
| Gold or gold ion | λ phage holin and a P22 | inducible | P22 gene 19 |
| | phage holin | | |
| Gold or gold ion | A λ phage holin and a P22 | inducible | P22 gene 15 |
| | phage holin | | |
| Gold or gold ion | A λ phage holin and a P22 | inducible | P22 gene 19 and P22 gene 15 |
| | phage holin | | |
| Gold or gold ion | A λ phage holin and a P22 | inducible | At least one λ phage endolysin and at least one P22 phage |
| | phage holin | | endolysin |
| Silver or silver ion | λ phage holin | inducible | At least one Cyanophage endolysin |
| Silver or silver ion | λ phage holin | inducible | At least one λ phage endolysin |
| Silver or silver ion | λ phage holin | inducible | P22 gene 19 |
| Silver or silver ion | λ phage holin | inducible | P22 gene 15 |
| Silver or silver ion | λ phage holin | inducible | P22 gene 19 and P22 gene 15 |
| Silver or silver ion | λ phage holin | inducible | At least one λ phage endolysin and at least one P22 phage |
| | | | endolysin |
| Silver or silver ion | Cyanophage holin | inducible | At least one Cyanophage endolysin |
| Silver or silver ion | Cyanophage holin | inducible | At least one λ phage endolysin |
| Silver or silver ion | Cyanophage holin | inducible | P22 gene 19 |
| Silver or silver ion | Cyanophage holin | inducible | P22 gene 15 |
| Silver or silver ion | Cyanophage holin | inducible | P22 gene 19 and P22 gene 15 |
| Silver or silver ion | Cyanophage holin | inducible | At least one λ phage endolysin and at least one P22 phage |
| | | | endolysin |
| Silver or silver ion | P22 gene13 | inducible | At least one Cyanophage endolysin |
| Silver or silver ion | P22 gene13 | inducible | At least one λ phage endolysin |
| Silver or silver ion | P22 gene13 | inducible | P22 gene 19 |
| Silver or silver ion | P22 gene13 | inducible | P22 gene 15 |
| Silver or silver ion | P22 gene13 | inducible | P22 gene 19 and P22 gene 15 |
| Silver or silver ion | P22 gene13 | inducible | At least one λ phage endolysin and at least one P22 phage |
| | | | endolysin |
| Silver or silver ion | A λ phage holin and a P22 | inducible | At least one Cyanophage endolysin |
| | phage holin | | |
| Silver or silver ion | λ phage holin and a P22 | inducible | At least one λ phage endolysin |
| | phage holin | | |
| Silver or silver ion | λ phage holin and a P22 | inducible | P22 gene 19 |
| | phage holin | | |
| Silver or silver ion | A λ phage holin and a P22 | inducible | P22 gene 15 |
| | phage holin | | |
| Silver or silver ion | A λ phage holin and a P22 | inducible | P22 gene 19 and P22 gene 15 |
| | phage holin | | |
| Silver or silver ion | A λ phage holin and a P22 | inducible | At least one λ phage endolysin and at least one P22 phage |
| | phage holin | | endolysin |

### II. Bacteria

Another aspect of the invention encompasses a gram negative bacterium comprising an integrated nucleic acid construct of the invention. For instance, in one embodiment, the invention encompasses a gram negative bacterium comprising an inducible promoter operably-linked to a nucleic acid encoding a first protein capable of forming a lesion in the cytoplasmic membrane of the bacterium and at least one endolysin protein. In another embodiment, the invention encompasses a gram negative bacterium comprising a first nucleic acid, wherein the first nucleic acid comprises a first inducible promoter operably-linked to a nucleic acid encoding a first protein capable of forming a lesion in the cytoplasmic membrane of the bacterium; and a second nucleic acid, wherein the second nucleic acid comprises a second promoter operably-linked to a nucleic acid encoding at least one endolysin protein.

In certain instances, the invention encompasses a gram negative bacterium comprising more than one integrated nucleic acid construct of the invention. For instance, the invention may encompass a gram negative bacterium comprising a first inducible promoter operably-linked to a nucleic acid encoding a first protein capable of forming a lesion in the cytoplasmic membrane of the bacterium, a second inducible promoter operably-linked to a different nucleic acid encoding a first protein capable of forming a lesion in the cytoplasmic membrane of the bacterium, and at least two endolysin proteins. In a further embodiment, the nucleic acid sequences encoding the endolysin proteins may be operably linked to a constitutive promoter.

Methods of making bacteria of the invention are known in the art. Generally speaking, a gram-negative bacterium is transformed with a nucleic acid contstruct of the invention. Methods of transformation are well known in the art, and may include electroporation, natural transformation, and calcium choloride mediated transformation. For more details, see FIGS. 1 and 3 and the Examples. Methods of screening for and verifying chromosomal integration are also known in the art.

In one embodiment, a method of making a bacterium of the invention may comprise first transforming the bacterium with a vector comprising, in part, an antibiotic resistance marker and a negative selection marker. Chromosomal integration may be selected for by selecting for antiobiotic resistance. Next, the antibiotic strain is transformed with a similar vector comprising the target genes of interest. Chromosomal integration of the target genes may be selected for by selecting for the absence of the negative marker. For instance, if the negative marker is sacB, then one would select for sucrose resistance. For more details, see Kang et al., J Bacteriol. (2002) 184(1):307-12, hereby incorporated by reference in its entirety.

Non-limiting examples of suitable gram-negative bacteria may include the proteobacteria, including alpha, beta, gamma, delta, and epsilon proteobacteria. Exemplary examples include bacteria that are used in industrial microbiology for the production of byproducts. Non-limiting examples may include Acetobacter, Acinetobacter, Agrobacterium, Alcaligenes, Azotobacter, Cyanobacteria such as Synechocystis, Erwinia, Escherichia, Klebsiella, Methylocococcus, Methylophilus, Pseudomonas, Ralstonia, Salmonella, Sphingomonas, Spirulina, Thermus, Thiobacillus, Xanthomonas, Zoogloea, and Zymomonas. In one embodiment, the gram-negative bacterium is an E. coli strain. In another embodiment, the gram-negative bacterium is a Cyanobacteria. In yet another embodiment, the gram-negative bacterium is a Synechocystis strain. In still another embodiment, the gram-negative bacterium is Synechocystis PCC 6803.

In one embodiment, a bacterium of the invention comprises a nucleic acid from Table A above.

### III. Methods

Yet another aspect of the invention encompasses a method for degrading the peptidoglycan layer of a bacterial cell wall. In one embodiment, the invention encompasses a method for degrading the peptidoglycan layer of a cell wall of a gram-negative bacterium. Generally speaking, the method comprises inducing the first promoter in a bacterium of the invention, such that the first protein is expressed. Methods of inducing a promoter are well known in the art. For more details when the promoter is induced by a metal or metal ion, see the Examples. The first protein, by forming lesions in the cytoplasmic membrane, allows the endolysin to degrade the peptidoglycan layer of a bacterial cell wall. The endolysin may be operably-linked to the first promoter, or alternatively, the endolysin may be operably-linked to a second promoter, as detailed in section I(a) above.

The second promoter may be an inducible promoter, or a constitutive promoter. In some embodiments, the second promoter is a constitutive promoter. In these embodiments, the endolysin(s) are expressed and accumulate in the cell, but are inactive because they do not have access to the peptidoglycan layer of the cell wall. After the induced expression of the holin(s), the endolysin(s) has access to the peptidoglycan layer of the cell wall, and subsequently, may degrade the peptidoglycan layer of the cell wall.

In other embodiments, the second promoter is an inducible promoter. The inducible promoter may be induced by a different compound or condition than the first promoter. In these embodiments, expression of the endolysin(s) may be induced first, with the subsequent induction of the holin(s) via the first promoter.

In certain embodiments, the peptidoglycan layer of the cell wall is substantially degraded in less than 12 hours, less than 10 hours, less than 8 hours, less than 7 hours, less than 6 hours, less than 5 hours, or less than 4 hours. In one embodiment, the peptidoglycan layer of the cell wall is substantially degraded in less than 6 hours.

After the peptidoglycan layer of a cell wall is degraded, the remaining cytoplasmic membrane may be further disrupted to release the cytoplasmic contents of the cell into the media.

### DEFINITIONS

The term “cell wall”, as used herein, refers to the peptidoglycan layer of the cell wall. Stated another way, “cell wall” as used herein refers to the rigid layer of the cell wall.

The term “operably-linked”, as used herein, means that expression of a gene is under the control of a promoter with which it is spatially connected. A promoter may be positioned 5′ (upstream) of a gene under its control. The distance between the promoter and a gene may be approximately the same as the distance between that promoter and the gene it controls in the gene from which the promoter is derived. As is known in the art, variation in this distance may be accommodated without loss of promoter function.

The term “promoter”, as used herein, may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. In some embodiments, activators may bind to promoters 5′ of the −35 RNA polymerase recognition sequence, and repressors may bind 3′ to the −10 RNA polymerase binding sequence.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples illustrate various iterations of the invention.

### Introduction

With the development of bacterial genetics, many bacteria have been genetically designed as bioreactors to produce numerous products of value, such as proteins, chemicals, drugs, and fuels. Generally, most of the valuable products are produced and accumulated inside the bacterial cells. After fermentation, the bacterial cell wall needs to be disrupted in order to facilitate product recovery from the bacterial biomass. The traditional cell processing techniques include physical or chemical cell breakage methods such as sonication, homogenization, pressure decompression, addition of hydrolytic enzymes and by solvent disruption and extraction. However, most of these methods require high energy inputs or raise environmental issues that reduce the overall utility of the process. The present invention thus is designed to avoid these additional costs by simply having the producing bacteria lyse themselves at the appropriate time to release the intracellular valuable products for easy and inexpensive recovery.

One important potential application of our invention is to facilitate lipid recovery from cyanobacterial biomass to produce biodiesel. Petroleum, on which our modern society was built and is now dependent, is a diminishing resource with increasing environmental, political, and economic disadvantages. Renewable biofuels from photoautotrophic cyanobacteria are promising alternatives to address these disadvantages by improving sustainability, increasing energy security and decreasing greenhouse gas emissions. Cyanobacteria are excellent organisms for the production of biofuel. Unlike algae, their bacterial genomes are relatively easy to manipulate. They are efficient at converting solar energy into lipids, and unlike corn or other energy crops they can be grown on non-arable land. For a cost balanceable and environmentally friendly lipid recovery from cyanobacterial biomass, a cell wall disruption process was genetically programmed into the genome of cyanobacteria by introducing controllable lysis genes from bacteriophages and controlling the expression of these genes to break up the cells whenever desired to initiate lipid recovery. By programming the lipid extraction process into the cyanobacterial genome, we hope to reduce the cost of biomass harvesting and avoid lipid extraction with hazardous organic solvents. After the induced self lysis of the cyanobacterial biomass, the intracellular lipids would be released and float to the top of the aqueous phase forming a lipid layer for easy lipid recovery.

Besides cell wall interruption for biofuel recovery, the technique will also release the proteins and carbohydrates in the cell, which can be used as valuable nutrients or animal feeds. The invention also establishes a technique to control some lethal genes that cannot be constitutively expressed in bacteria.

### Materials and Methods

### A. Cyanobacterial Strains, Culture Media and Growth Conditions

Mutant strains were developed from Synechocystis sp. PCC 6803. Table 1 lists the Synechocystis strains used or developed for the Ni²⁺ inducing lysis system and the DNA vectors for construction of these strains. Table 2 lists the primer sequences used in construction of the vectors.

Synechocystis and mutant strains were grown at 30° C. in modified BG-11 medium with a supplement of 1.5 g/l NaNO₃ (Rippka, Derulles et al. 1979) and bubbled with a continuous stream of filtrated air under continuous illumination (50 μmol of photons per m² per s) and buffered with 10mM TEM-NaOH (pH 8.0). For growth on plates, 1.5% (wt/vol) agar and 0.3% (wt/vol) sodium thiosulfate were added to BG-11 agar. BG-11 medium was supplemented with 50 μg of kanamycin per ml for Km^{R} strains. The E. coli strain DH5α was grown at 37° C. on 1.5% (wt/vol) LB agar (Bertani 1951) for plasmid constructions. When using the E. coli cells to replicate the plasmids harboring the lysis genes, the cells were grown at 20° C. in LB broth and agitated by slow rotation (30 rpm) to avoid lysis.

TABLE 1
Plasmids and Synechocystis strains used or developed for the Ni²⁺ inducing lysis system

| Vectors | Vector Description ^{a} | Strains | Strain Description ^{a} |
|---|---|---|---|
| pΨ101 | For the construction of SD101, | SD101 | ΔnrsBA::13 15 19 Km^{R} |
| | ΔnrsBA::13 15 19 Km^{R} | | A preliminary strain to test the feasibility for controllable |
| | | | lysis. |
| pΨ102 | For the construction of SD102, | SD102 | ΔnrsBAC::13 Km^{R} sacB |
| | ΔnrsBAC::13 Km^{R} sacB | | An intermediate strain containing a Km^{R}-sacB cassette for |
| | | | further insertion. |
| pΨ103 | For the construction of SD103, | SD103 | ΔnrsBAC::13 |
| | ΔnrsBAC::13 | | A strain with only one holin gene 13 from P22. |
| pΨ121 | For the construction of SD121, | SD121 | ΔnrsBAC::13 19 15 |
| | ΔnrsBAC::13 19 15 | | Strategy 1, P22 lysis cassette was inserted. |
| pΨ122 | For the construction of SD121, | SD122 | ΔnrsBAC::S R Rz |
| | ΔnrsBAC::S R Rz | | Strategy 1, λ lysis cassette was inserted. |
| pΨ123 | For the construction of SD123, | SD123 | ΔnrsBAC::13 TP4 P_{psbAll}19 15 |
| | ΔnrsBAC::13 TP4 P_{psbAll} 19 15 | | Strategy 2, holin 13 was controlled by Ni²⁺, endolysin genes |
| | | | 19 and 15 were transcribed by a constitutive promoter |
| | | | (P_{psbAll}). A transcriptional terminator was inserted to |
| | | | eliminate interference. |
| pΨ124 | For the construction of SD124, | SD124 | ΔnrsBAC::13 TP4 P_{psbAll} 19 15 (—) |
| | ΔnrsBAC::13 TP4 P_{psbAll} 19 15 (—) | | Strategy 2, the P_{psbAll}19 15 was inserted in a different |
| | | | orientation of 13. |
| pΨ125 | For the construction of SD125, | SD125 | ΔnrsBAC::13 S TP4 P_{psbAll} 19 15 |
| | ΔnrsBAC::13 S TP4 P_{psbAll} 19 15 | | An intermediate strain for SD126 |
| pΨ126 | For the construction of SD126, | SD126 | ΔnrsBAC::13 S TP4 P_{psbAll} 19 15 slr1704::Km^{R} sacB |
| | slr1704::Km^{R} sacB | | An intermediate strain for SD127 |
| pΨ127 | A PCR fragment consisting of flanking | SD127 | ΔnrsBAC::13 S TP4 P_{psbAll} 19 15 slr1704::P_{psbAll}R Rz |
| | regions and P_{psbAll}R Rz for SD127, | | Strategy 3, a double mutant incorporating P22 and A lysis |
| | slr1704::P_{psbAll}R Rz | | genes. |

^{a} nrsRS, nickel sensing and responding genes; P_{nrsB}, the nickel inducible promoter; nrsBACD, nickel resistance genes; 13, 19 and 15, Salmonella phage P22 genes 13 (holin), 19 (endolysin) and 15; S, R and Rz, coliphage λ genes S (holin), R (endolysin) and Rz;
Km^{R}, kanamycin resistance cassette; sacB, sacB gene, which is lethal for cyanobacteria in the presence of sucrose; P_{psbAll}, promoter of Synechocystis gene psbAll; TP4, transcriptional terminator from cyanophage Pf-WMP4.

TABLE 2
Primers used in the construction

| | | |
|---|---|---|
| Primer Name | Sequences (5′ to 3′) | SEQ ID NO |

construction of pψ101

| | | |
|---|---|---|
| SynL-S-SacI | GCGAGCTCCAGACGACTACGGGCAAAG | SEQ ID NO: 13 |
| SynL-A-to-P22 | ATGTTTTTCTGGCATCACACCACCTCAAATTGGG | SEQ ID NO: 14 |
| P22-S-to-SynL | TTGAGGTGGTGTGATGCCAGAAAAACATGATCT | SEQ ID NO: 15 |
| P22-A-SacII | GACCGCGGTTATTTTAAGCACTGACTCC | SEQ ID NO: 16 |
| KR-S-SacII(-) | GGCCGCGGAAAGCCACGTTGTGTCTCA | SEQ ID NO: 17 |
| KR(-)-A-to-Syn | ACCCCCTGGGGCAGAAAGCCACGTTGTGTCTCA | SEQ ID NO: 18 |
| SynR-S-to-KR(-) | ACAACGTGGCTTTCTGCCCCAGGGGGTTTCTTGA | SEQ ID NO: 19 |
| SynR-A-BamHI | GGGATCCGTTGGTTAGCCAAGAGAATC | SEQ ID NO: 20 |

construction of pψ102

| | | |
|---|---|---|
| P2213-A-NdeI | GACATATGTTACTGCTGATTTGCATCATCGA | SEQ ID NO: 21 |
| SynR-A-SacII | GACCGCGGAACTAATGGCTTGGGCTAGGTATA | SEQ ID NO: 23 |

construction of pψ121

| | | |
|---|---|---|
| SynL-S-KpnI | GAGGTACCGCCAATTGCAGACGACTACG | SEQ ID NO: 24 |
| SynR-S-XbaI | GATCTAGACACATTGCTCCTTTTGTGCGTAA | SEQ ID NO: 25 |
| SynR-A-SacII | GACCGCGGAACTAATGGCTTGGGCTAGGTATA | SEQ ID NO: 26 |
| Syn-right-A-SphI | AGGCATGCGTTGGTTAGCCAAGAGA | SEQ ID NO: 27 |
| P22-A-to-F1 | GCACAAAAGGAGCAATGTGTTATTTTAAGCACTGACTCC | SEQ ID NO: 28 |
| F1-S-to-P22 | TCAGTGCTTAAAATAACACATTGCTCCTTTTGTGCG | SEQ ID NO: 29 |
| SynR-A-F2 | CAAACTAATGGCTTGGGCTAGGTATAGCT | SEQ ID NO: 30 |

construction of pψ122

| | | |
|---|---|---|
| F1-A-to-LMD | CATGTTTTTCTGGCATCACACCACCTCAAATTGGG | SEQ ID NO: 31 |
| LMD-S-to-F1 | AGGTGGTGTGATGCCAGAAAAACATGACCT | SEQ ID NO: 32 |
| LMD-A-to-F2 | ACAAAAGGAGCAATGTGCTATCTGCACTGCTCATTAATA | SEQ ID NO: 33 |
| F2-S-to-LMD | AGTGCAGATAGCACATTGCTCCTTTTGTGCGT | SEQ ID NO: 34 |
| SynR-A-SacII | GACCGCGGAACTAATGGCTTGGGCTAGGTATA | SEQ ID NO: 35 |

construction of pψ123 and pψ124

| | | |
|---|---|---|
| tP4-S | ATCATATGAAGACAAACGAAAGCCCCCACCTAGCGTCATGCC | SEQ ID NO: 36 |
| | GGGTGGGGGCTTTTTCATCTGCAGTA | |
| tP4-A | TACTGCAGATGAAAAAGCCCCCACCCGGCATGACGCTAGGTG | SEQ ID NO: 37 |
| | GGGGCTTTCGTTTGTCTTCATATGAT | |
| tP4-A-PstI | CTGCAGATGAAAAAGCCCCCACC | SEQ ID NO: 38 |
| pA2-S-BamHI | GAGGATCCTAATTGTATGCCCGACTATT | SEQ ID NO: 39 |
| pA2-A-to-P2219 | ACTGCTGATTTGCATCATTTGGTTATAATTCCTTATG | SEQ ID NO: 40 |
| P2219-S-to-pA2 | GAATTATAACCAAATGATGCAAATCAGCAGTAACGG | SEQ ID NO: 41 |
| P2215-A-BamHI | GAGGATCCTTATTTTAAGCACTGACTCCT | SEQ ID NO: 42 |
| lambdaS-S-NdeI | GACATATGCCAGAAAAACATGACCTGT | SEQ ID NO: 43 |

construction of pψv126

| | | |
|---|---|---|
| 52F1-S-HindIII | AGaagcTTTGTGGCCCAACAATTGGT | SEQ ID NO: 44 |
| 52F2-A-EcoRI | GTGAAtTCTGTAAGCAGTTAGAGTGGCCC | SEQ ID NO: 45 |
| S2-segS-400 | CGGTCTACTCCGGTTAAATCCCCTAACG | SEQ ID NO: 46 |
| S2-segA-400 | CCACAGCCCCAACAATAAGCAAGAT | SEQ ID NO: 47 |

construction of pψv127

| | | |
|---|---|---|
| lambdaS-S-NdeI | GACATATGCCAGAAAAACATGACCTGT | SEQ ID NO: 48 |
| lambdaS-S-NdeI-RBS | GACATATGAGGAGGTGTGATGCCAGAAAAACATGACC | SEQ ID NO: 49 |
| pA2-A-to-R | ACTGCTGATTTGCATCATTTGGTTATAATTCCTTATG | SEQ ID NO: 50 |
| R-S-to-pA2 | GAATTATAACCAAATGATGCAAATCAGCAGTAACGG | SEQ ID NO: 51 |

B. Strategies for Introducing Controllable Lysis Genes into the Syenchocvstis genome

For most phages, infection cycle terminates with strictly programmed lysis of the host by phage-encoded proteins: lysozyme (also called endolysin or lysin), and the holin, a small membrane protein that triggers the function of lysozyme (Young 1992). Lysozymes are a set of muralytic enzymes that attack at least one of three covalent linkages (e.g. glycosidic, amide and peptide) of the peptidoglycans that maintain the integrity of the cell wall (Loessner 2005). Holins are a group of small membrane proteins that produce non-specific lesions (holes) in the cytoplasmic membrane from within and allow the lysozyme to gain access to the cell wall and trigger the lysis process. Holins are non-specific and independent of host proteins (Young et al 2002). For example, the λ S gene holin S also functions efficiently in yeast.

Three strategies were devised to control the lysis genes introduced in Synechocystis PCC 6803 cells. Strategy 1 places the lytic operon including the holin and lysozyme genes together and under the control of an inducible element. Strategy 1 uses the lysozymes from P22 (in SD121) and λ (in SD122), respectively, to test the lysing abilities of lysozymes from different bacteriophages.

Strategy 2 is to overexpress the lysozyme genes under a strong constitutive Synechocystis PCC 6803 promoter P_{psbAll} (Shibato, Agrawal et al. 2002), while restricting the control of the expression of the holin gene (P22 13). This strategy is expected to cause severe and speedy damage to the cell wall. Before induction of the holin gene, however, the lysozymes accumulate in the cell, but cannot reach their cell wall substrate. Once the holin is expressed, the cells would produce non-specific lesions (holes) in the membrane from within, allowing the lysozyme to gain access to the cell wall and trigger the lysis process.

Strategy 3 is to incorporate the lysis genes from other phages with P22 lysis genes, such as coliphage λ lysis genes S R Rz, with the assumption that different lysozymes attacking different bonds in the cell envelope will result in a faster lysis rate.

### C. Molecular and Gene Procedures

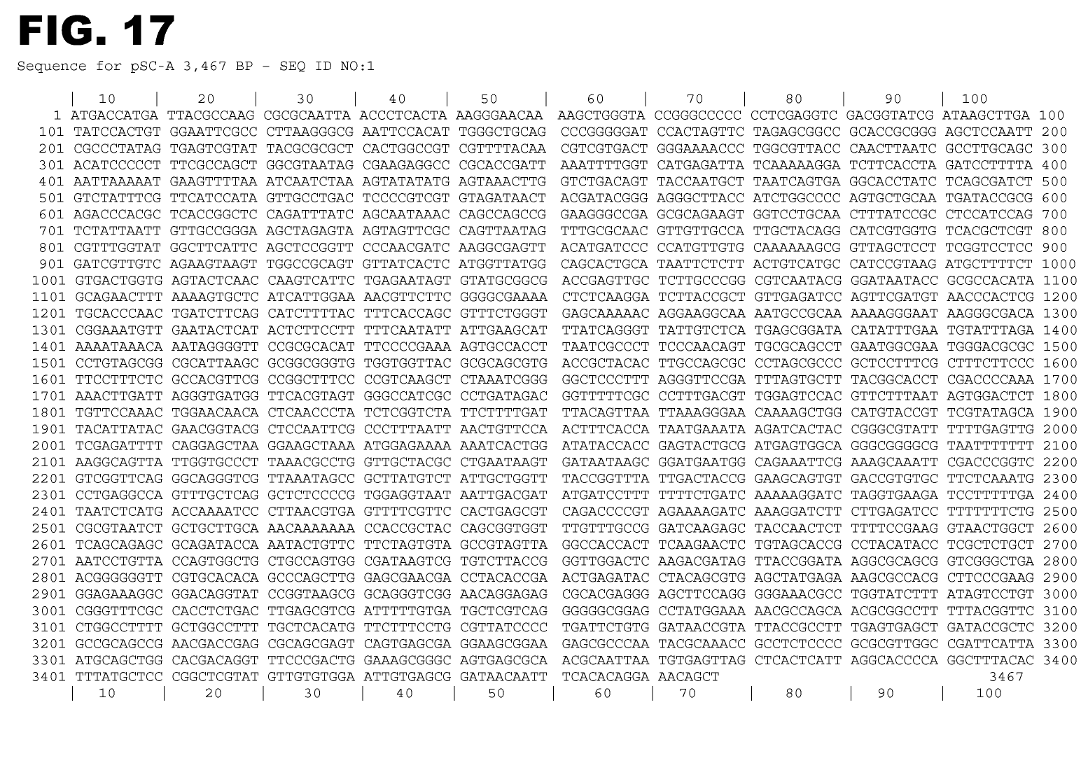

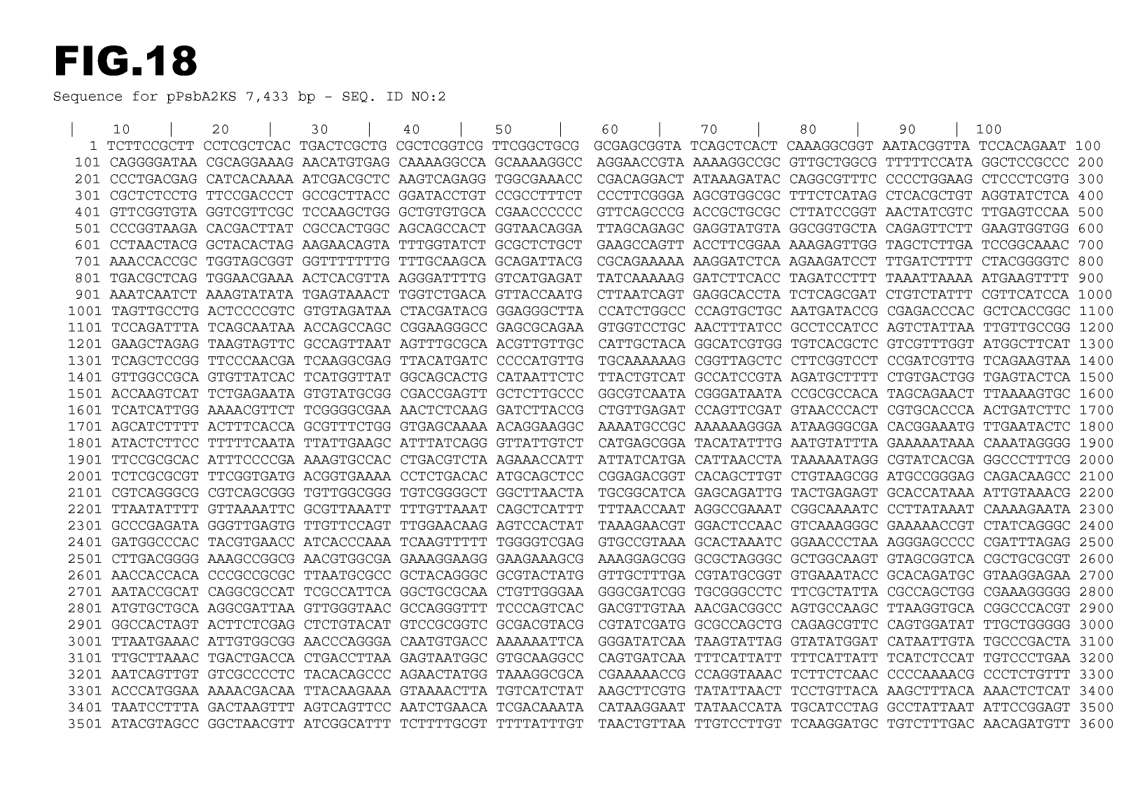

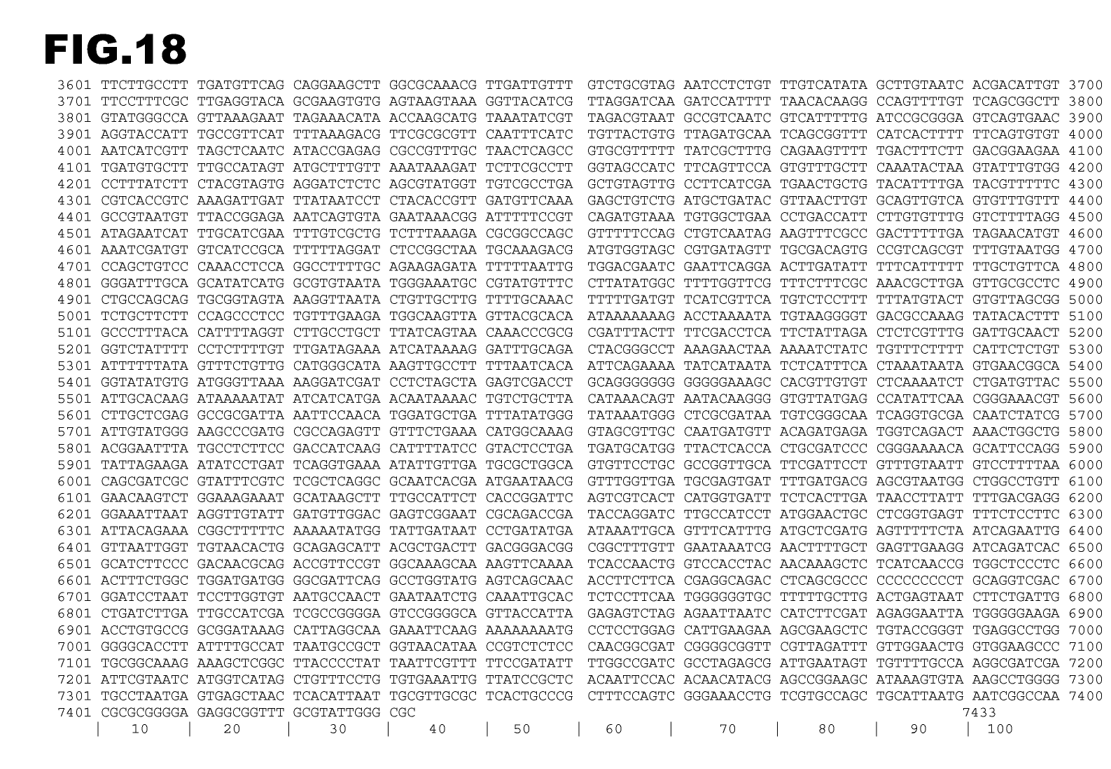

Unless indicated otherwise, standard DNA methods (Sambrook, Fritsch et al.) were used. In some plasmids, mutagenesis was created by the PCR overlap extension method (Warrens, Jones et al. 1997). Plasmids and constructions used in this study are listed in Table 1. The primers used in the constructions are listed in Table 2. The flanking sequences for double crossover recombination were cloned into pSC-A (FIG. 17). Using PCR, the lysis gene cassettes were amplified from a Salmonella phage P22 lysate and an E. coli phage λ lysate. The Km^{R} cassette was cloned from pUC4K (Oka, Sugisaki et al. 1981). The sacB cassette was cloned from pRL271 (Black, Cai et al. 1993). All the plasmid constructions were confirmed by DNA sequence analysis performed in the DNA Lab, School of Life Sciences at Arizona State University.

### D. Transformation of Synechocystis

The cyanobacterium Synechocystis sp. PCC 6803 is transformable at high efficiency and integrates DNA by homologous double recombination. General conditions for transformation of Synechocystis sp. PCC 6803 have been optimized. (Kufryk, Sachet et al. 2002) However, in this example transformation procedures were modified, because the suicide vectors containing lysis genes were found to be lethal when inserted into Synechocystis cells. This was the first evidence that Salmonella phage P22 and E. coli phage λ genes are expressed in Synechocystis PCC 6803.

i. Transformation of Suicide Vectors Containing Kanamycin Resistance-SacB Cassette

50 ml of exponential growth Synechocystis cultures (OD_{730 nm} of 0.2 ˜0.5) were gently harvested by a low force centrifugation (3000×g, 5 min), and concentrated to a density of OD_{730 nm} of 1.0 by resuspension in the modified BG-11 medium. A volume of 0.5 ml concentrated Synechocystis cells were mixed with 2 μg suicide vector DNA (e.g. pψ102), and incubated under the cyanobacterial culture conditions for 5 hours. Then the mixtures were plated onto a filter membrane (Whatman PC MB 90MM 0.4 μM) layered on a BG-11 agar plate. After segregation on the BG-11 plate for about 24 hours, the membrane carrying the cyanobacteria was transferred onto a BG-11 plate containing 50 μg/ml of kanamycin for transformation selection. Generally, the colonies appeared 5 days later. Then the colonies were transferred onto a kanamycin BG-11 plate for segregation.

ii. Segregation

In the cells of Synechocystis PCC 6803, there are multiple copies of chromosomal DNA. When a Synechocystis is transformed using double crossover recombination, only one chromosome is involved in the initial recombination event. Essentially, the selected colonies are genotypic mixtures of cells, so isolating colonies derived from single cells obtained after growth of the segregating clone is necessary for obtaining a genetically pure recombinant strain.

For colonization of recombinant cells. cells in the segregated culture are diluted in BG-11 medium, vortexed and spread onto BG-11 plate for growing from single cells. Finally, restreaking of suspended cells on selective plates yields colonies derived from single cells in which all chromosomes possess the identical desired genotype. This can be verified by using PCR.

iii. Transformation with Markerless Constructs

To remove the antibiotic-resistance selection marker for further genetic manipulation, recombinant strain SD102 was transformed using markerless suicide vectors. The Km^{R}-sacB cassette is replaced in the recombinants with the lysis genes. With the removal of sacB, recombinants are able to grow on BG-11 plates containing 4.5% sucrose, while the untransformed cells cannot. Cells are also unable to grow on BG-II plates containing kanamycin, to which the original recombinant was resistant. The following is the optimized protocol.

A cell culture is grown into exponential phase at an OD_{730 nm} of 0.6, about 10⁸ cells/ml.

The cell culture (50 μl) is mixed with 200 ng of transforming DNA (e. g. pψ112 or PCR product), resulting in a final DNA concentration of 4 μg/ml. A control without DNA addition is also necessary.

After 5 hours of incubation under normal growth conditions (20 μmol photons m⁻² sec⁻¹, 30° C.), the whole transformation mixture is inoculated into 3 ml BG-11 media, grown under normal conditions for 5 days for segregation.

Sucrose resistance selection. 200 μl of culture is spread onto a BG-11 plate containing 4.5% sucrose (w/v), and grown under normal conditions. The incubation might take 8 days or more, before green colonies grow big enough for segregation.

Segregation. The cells growing on the sucrose BG-11 plate are inoculated into 3 ml BG-11 media, and grown for one week under normal conditions for full segregation.

Colonization. The cells with the correct genetic replacement need to be isolated as a genetically pure strain. Cells in the segregated culture are diluted in BG-11 medium, and votexed for 3 min. A dilution containing about 300 cells is spread onto a 4.5% sucrose BG-11 plate, and cultured under normal growth conditions for 5 days. The colonies growing after colonization can be regarded as genetically pure strains.

Confirmation of Replacement. The colonies on the sucrose BG-11 plate should be identified by PCR to confirm the insertion/deletion and segregation status. Cells in a colony are resuspended in 2 μl water and transferred into a 200-μl PCR tube. The cell suspension in the PCR tube is frozen at −80° C. for 2 min, and then thawed in a 60° C. water bath. This freeze-thaw cycle needs to be performed two times. 1 μl frozen-thawed cell suspension is used as the PCR template for a 30 μl PCR system including the primers specific for the inserted DNA or the deleted region.

Stock. The cells of the positive colony are suspended from plates, transferred in glycerol-BG-11 solution (15% glycerol, v/v), distributed into at least four tubes and frozen at −80° C.

### E. PCR Identification of the Introduced Lysis Genes

The integration of introduced lysis genes in the genetically pure recombinant strains should be identified by PCR using specific primers. Recombinant cells that were freeze-thawed were used as PCR templates. Briefly, 200 μl cultures (with an OD₇₃₀ of 0.1˜0.5) of recombinant cells were harvested in 250 μl PCR microcentrifuge tubes. The cell pellets were frozen at −80° C. for 3 min, and then thawed in a 60° C. water bath. This freeze-thaw cycle was performed three times. 1 μl frozen-thawed cell pellets was used as PCR template for a 30 μl PCR system. PCR is used to demonstrate that the recombinant strain is totally absent of the parental strain DNA sequence and PCR positive for the inserted sequence.

The positive colonies should be suspended from plates, transferred in glycerol-BG11 solution (15% glycerol, v/v), distributed into at least four tubes and frozen at −80° C. for stocking.

### F. Genetic Stability Test

This method tests the stability of the lysis genes in the purified SD strains after 75 generations to make sure that these strains are genetically stable.

200 ml SD cultures at the initial OD_{730 nm} of 0.01 are grown in the bubbling flasks with aeration. When the culture OD_{730 nm} reached 1.2, the culture would be subcultured by a 1:1000 dilution in prewarmed medium. The segregation status and insertion sequences were verified using PCR for different subcultures.

### G. Resistance Mutation Frequency Test

This method is to test the mutation frequency to Ni²⁺ resistance caused by spontaneous mutation. Due to spontaneous mutation, some Ni²⁺ resistant individuals would appear in the population as the culture grew. During the 75-generation culture period, Ni²⁺ resistance frequencies were evaluated by the surviving rates of the culture samples on Ni²⁺ containing BG-11 plates. The following is the protocol for determining the mutation rates to Ni²⁺ resistance for each strain. Adjust the OD_{730 nm} of each subsample to 0.2, if necessary. Dilute the liquid BG-II culture by 1:10⁴ or 1:10⁵. Plate 100 μl undiluted subsample on the BG-11 plates containing 7 or 20 μM Ni²⁺, and 100 μl diluted cultures on BG-11 plates without Ni²⁺. After 5 days culture under normal conditions, count the surviving colonies on Ni plates (Nn) and colonies on BG-11 plates (Nb). The Ni²⁺ resistance mutation frequency for this culture (Rf) was calculated from Nn, Nb and the dilution rate. Generate a curve of Rf verses number of generations; the slope represents the mutation rate.

### H. Recombinant Growth Rate Measurements

The growth rates of the recombinant strains were measured in triplicate 300 ml liquid cultures with air bubbling aeration at a photon flux density of 50 mmol of photons·m⁻²·s⁻¹ at 30° C. At 24-hour time intervals, cultures were sampled and cell density was counted in a haemocytometer.

### I. Inducible Lysis Responses

The inducible cell lysis responses of recombinant strains were tested by addition of Ni²⁺ to the culture. The initial culture concentrations were adjusted to 10⁸ cells/ml (OD_{730 nm} ˜0.6). After NiSO₄ was added to the cultures with a final concentration of 7.0, 20, and 50 μM Ni²⁺, lysis responses were inspected by measuring decline in colony formation units (CFU). Briefly, after dilution (10⁻⁴ to 10⁻¹, according to culture density), 0.02 μl, 1 μl and 10 μl of dilutions were plated onto BG11 agar plates. After 5 days culture, colonies appearing on the plates were counted as viable cells and the titers were calculated.

### J. TEM Sample Preparation

The effects of lysozyme on cyanobacterial cell walls were illustrated by transmission electron microscope (TEM). A specific cell fixation procedure for Synechocystis sp. PCC 6803 and mutant strains is shown below. All steps were at room temperature unless noted. Initial steps may be done in Eppendorf tubes.

For primary fixation, cells in suspension were treated with 2% glutaraldehyde in 50 mM KH₂PO₄—K₂HPO₄ buffer, pH 6.8 for 2 h or overnight at 4° C. The fixed cells were sedimented by centrifugation, the fixative decanted, cells resuspended in approx 1 ml of the same buffer, followed by inversion of the tube for a few minutes. Cells were then washed three times by sedimentation and resuspension.

Solidify cells in agarose, pellet and decant wash buffer. Resuspend in approx 50-100 μl of KH₂PO₄—K₂HPO₄ buffer. Pipet cells from tube and put onto a small piece of parafilm. Add equal vol of 2% agarose (melt, then cool to near-solidification point). Pipet cell-agarose mixtures. Cut into 4-5 small chunks with lancet or shaver and transfer to a glass vial, wash with buffer, allow to sit for 15 min. Repeat wash two times.

Secondary fixation, for lipid fixation, is achieved in 1% osmium tetroxide in the same buffer for 2 hr. Remove 2nd fixation solution. Wash 3 times with buffer, then 3 times with de-ionized H₂O, 15 min per step.

Uranyl blocking stain is achieved by treatment with 2% aqueous uranyl acetate for 2 h at room temperature or overnight at 4° C. Wash 3 times with H₂O, 15 min each. Remove uranyl acetate. Dehydrate samples through the following ethanol series, 5-10 min each step: 20%, 50%, 75%, 95%, and 100% EtOH 3 times, then in 1:1 EtOH:acetone 2 times.

Lead blocking stain. Incubate cells 1h at room temperature in a saturated solution of lead acetate in 1:1 EtOH:acetone. Wash samples 2 times for 15 min in 1:1 EtOH:acetone, then 2 times for 15 min each in acetone.

Infiltrate with increasing epoxy resin (Spurr's resin, firm mixture) series, 25% increments, using 100% resin 3 times. Place vials on rotary wheel during all these steps. Specifically, 25% and 50% steps for a minimum of 4 h; 75% and 100% steps for 6 h.

Polymerization. After 3rd 100% resin step, embed cell-chunks in flat molds using fresh resin. Put in oven at 60° C. for 24-36 h. The polymerized molds need to be trimmed first and cut into sections in a microtome. Sections on grids can be post stained if necessary, and then can be checked under TEM.

### K. Sample Preparation for Fluorescence Microscopy

The lysing cells after 7 μM Ni²⁺ induction were stained with 5 μM SYTOX Green nucleic acid stain (Invitrogen Molecular Probes, Inc. OR, USA) (Roth, Poot et al. 1997) for 5 min and observed under an Axioskop40 fluorescence microscope (Zeiss, Germany). At least 400 cells were counted on the pictures taken for different samples and for time points before and after 7.0 μM Ni²⁺ addition.

### Example 1

Example 1 demonstrates a method to construct a test strain containing inducible phage P22 lysis genes and a selective kanamycin-resistance marker (Km^{R}), and evidence that the lysis genes from Salmonella and E. coli bacteriophages are able to lyse Synechocystis cells after induction.

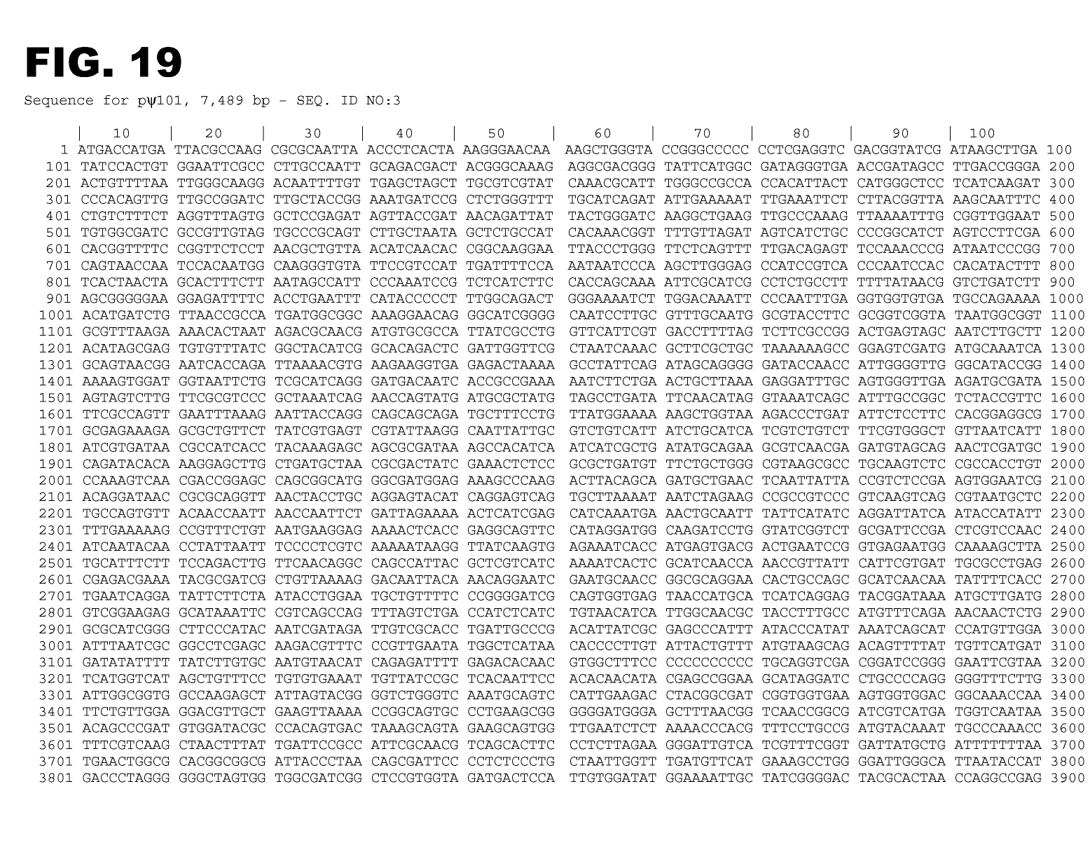

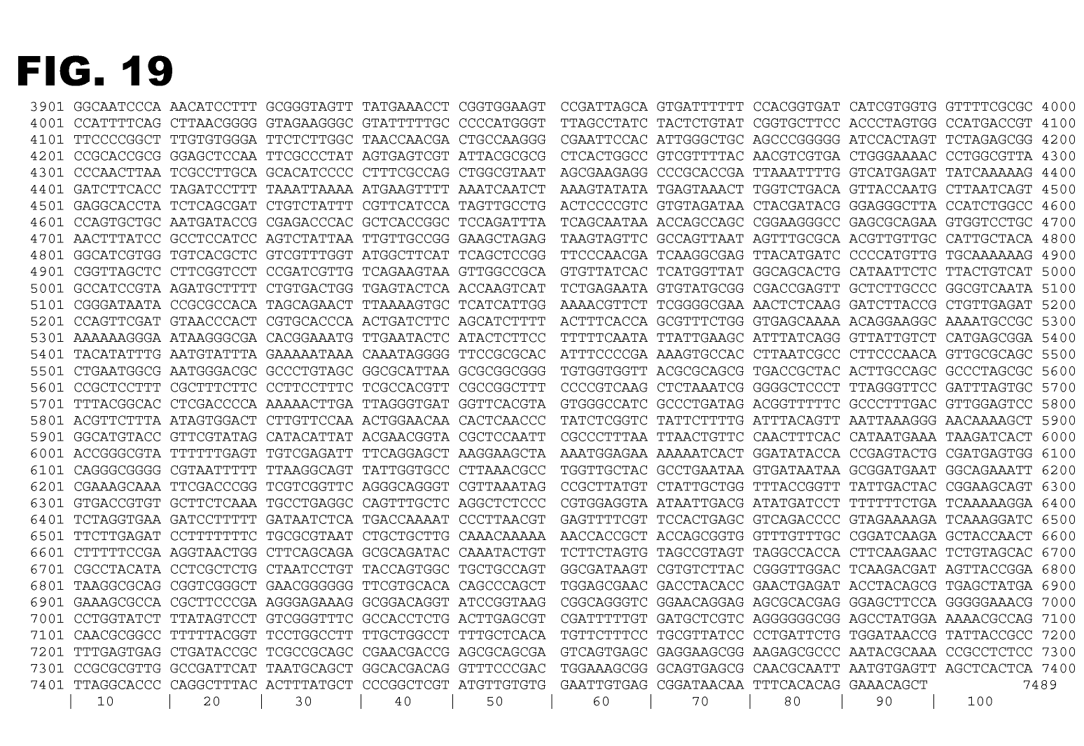

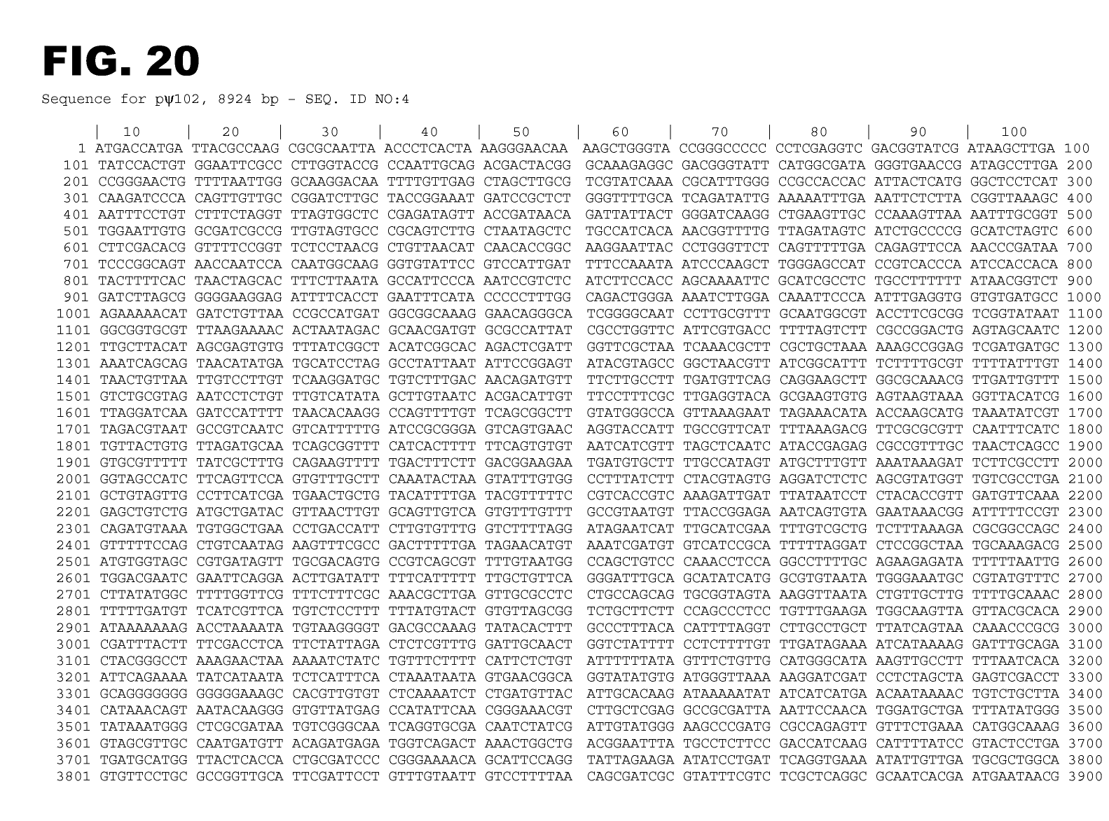

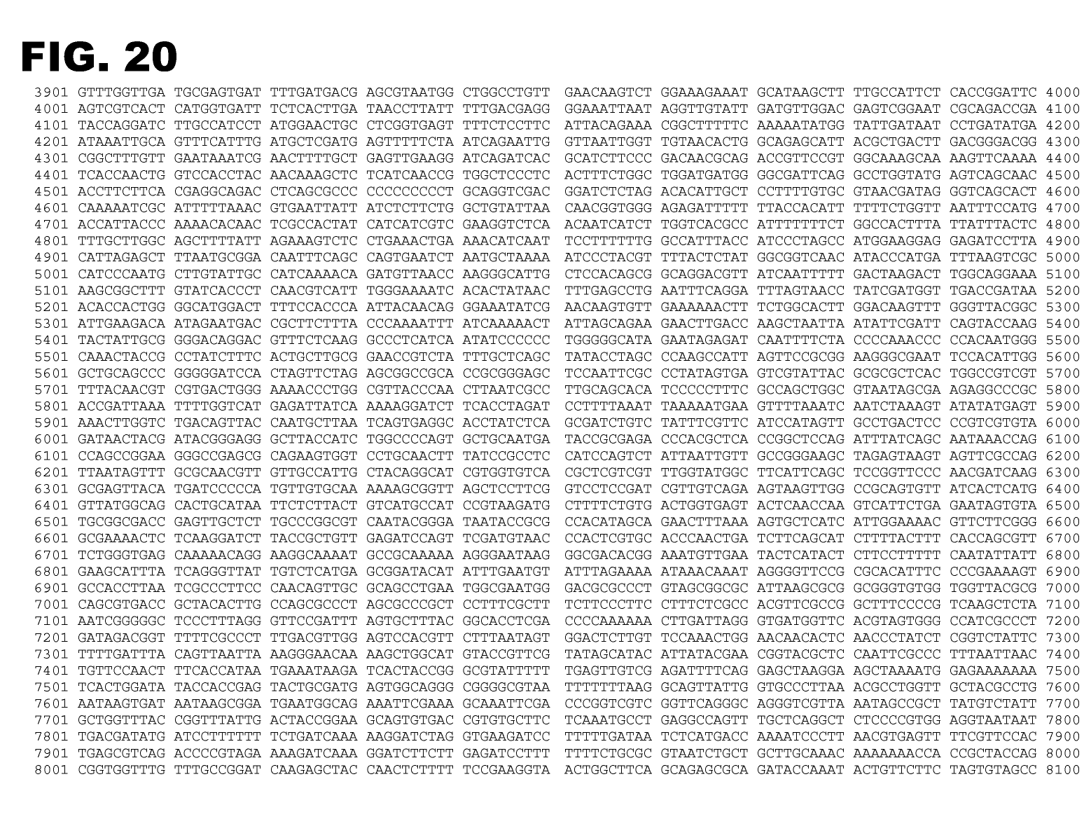

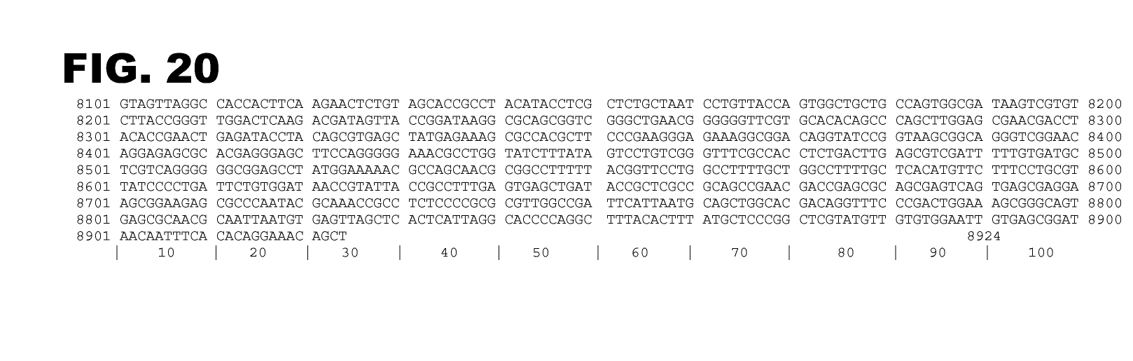

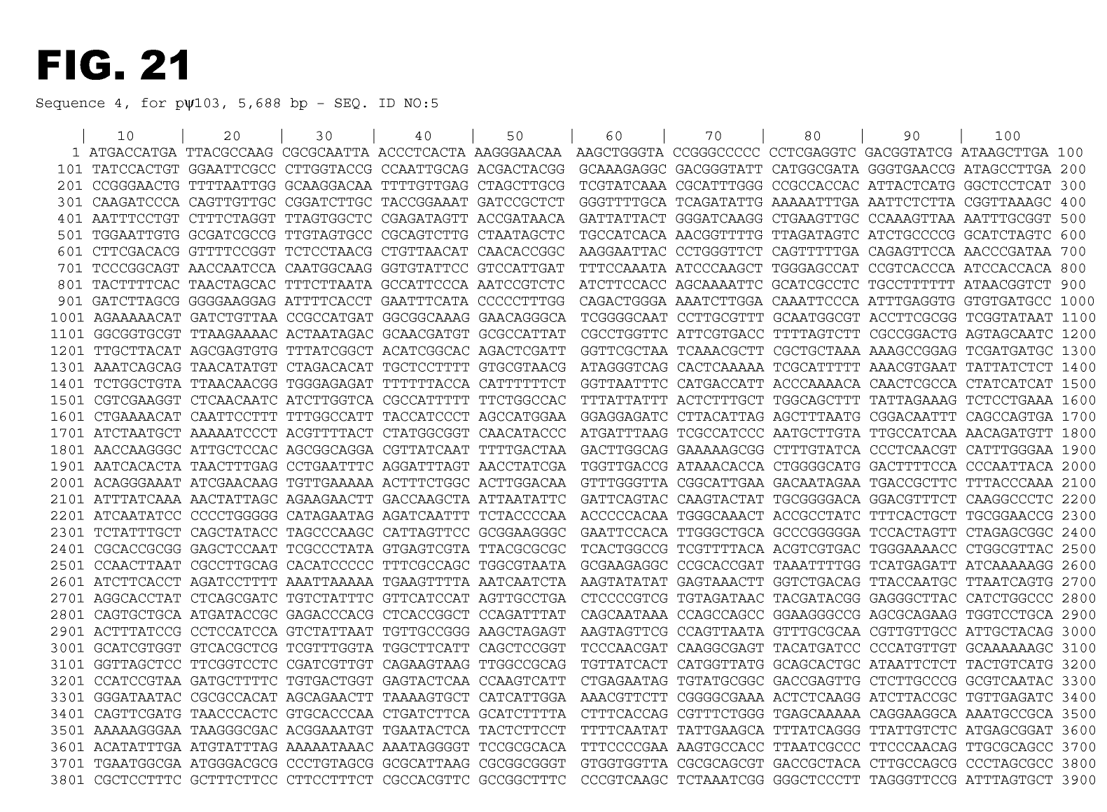

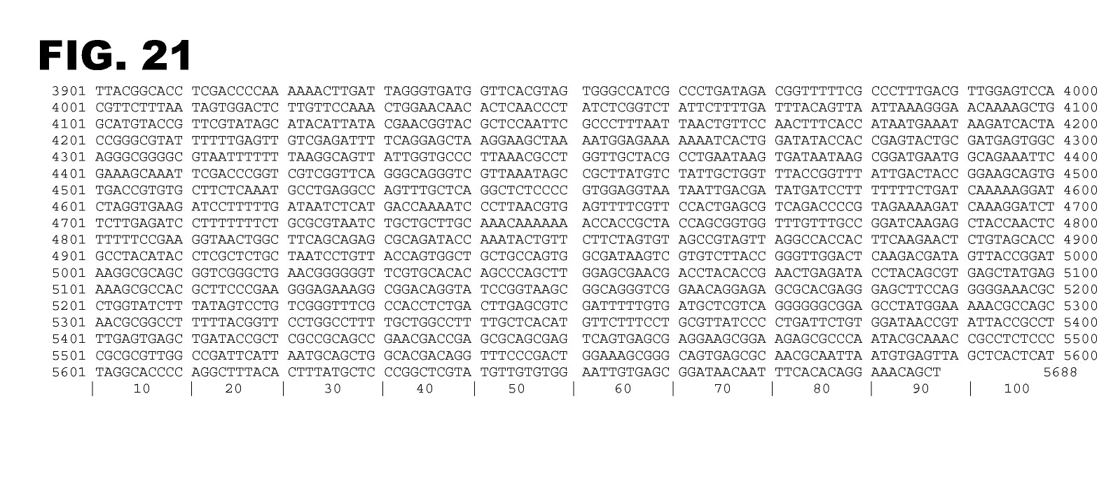

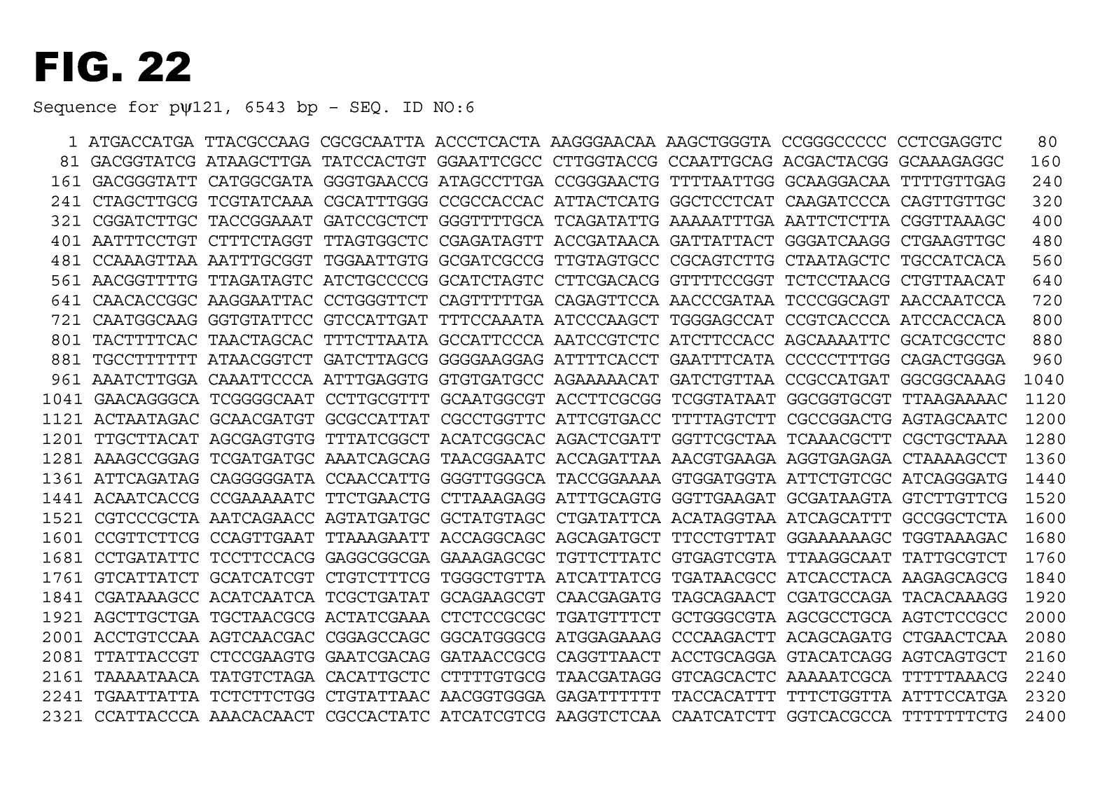

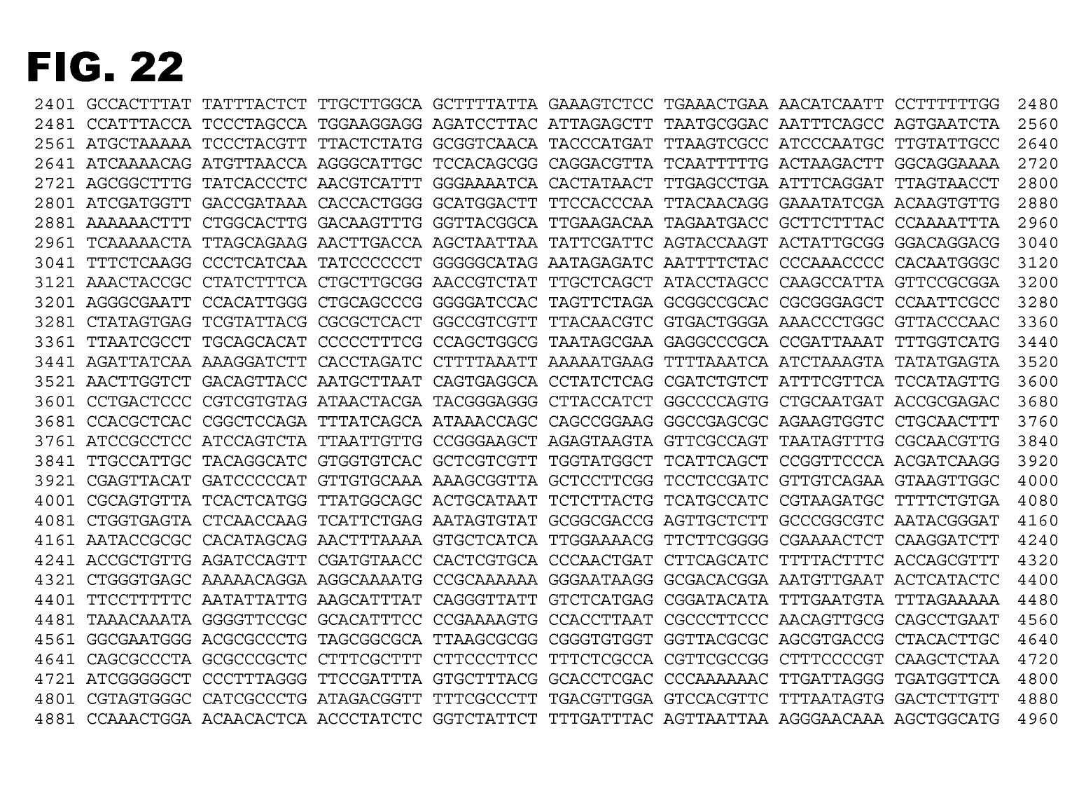

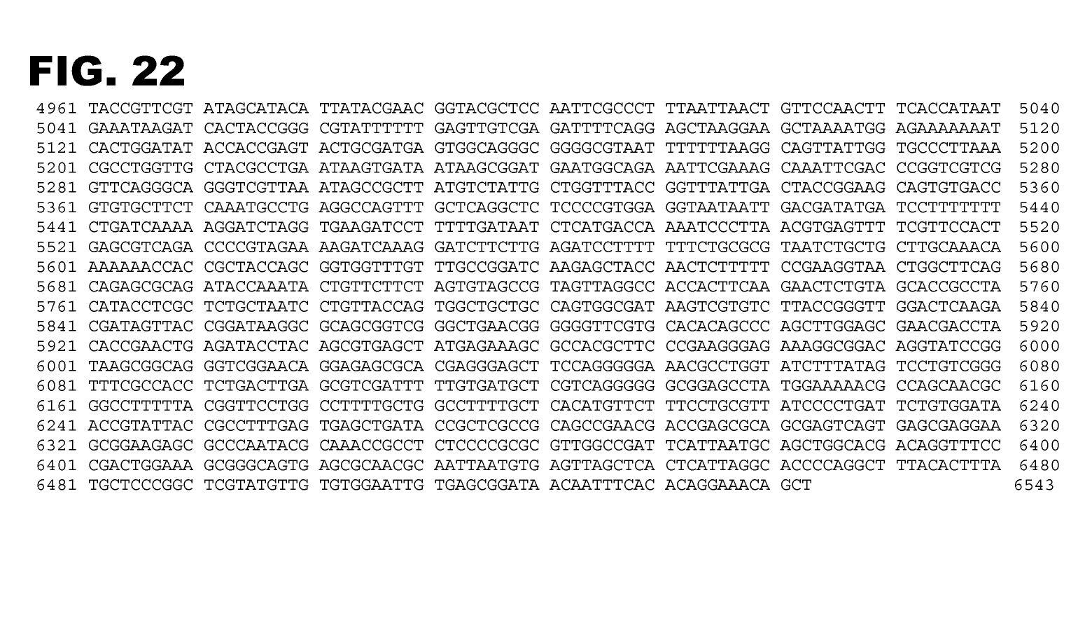

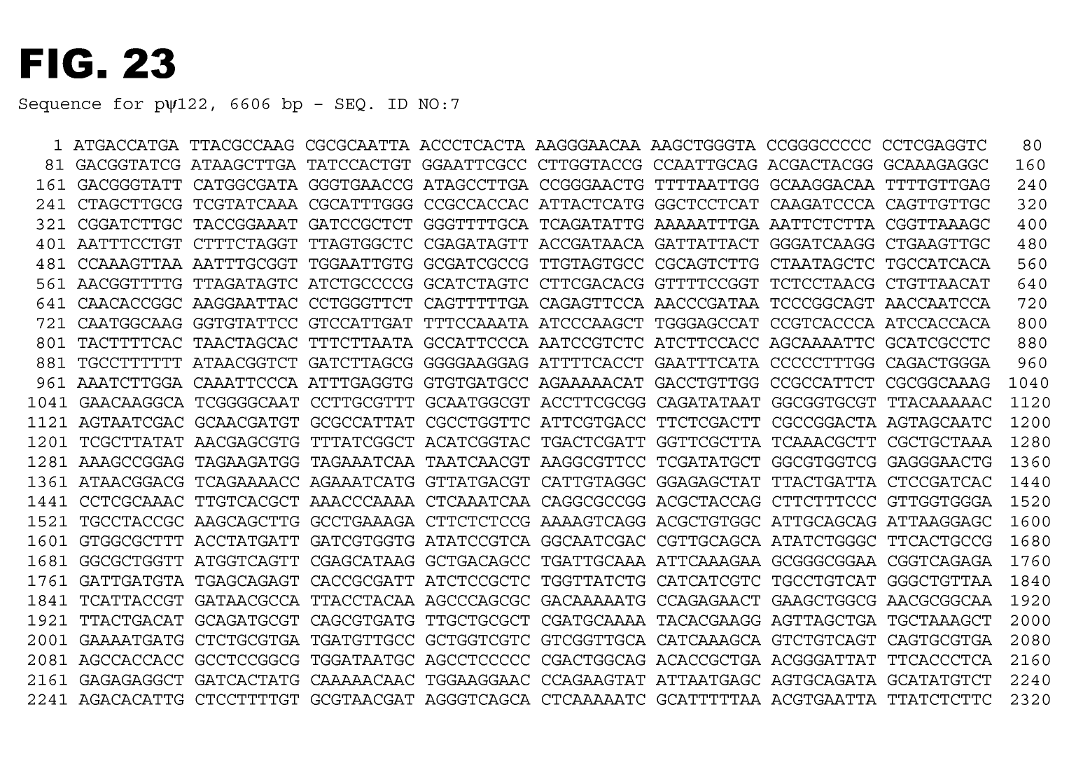

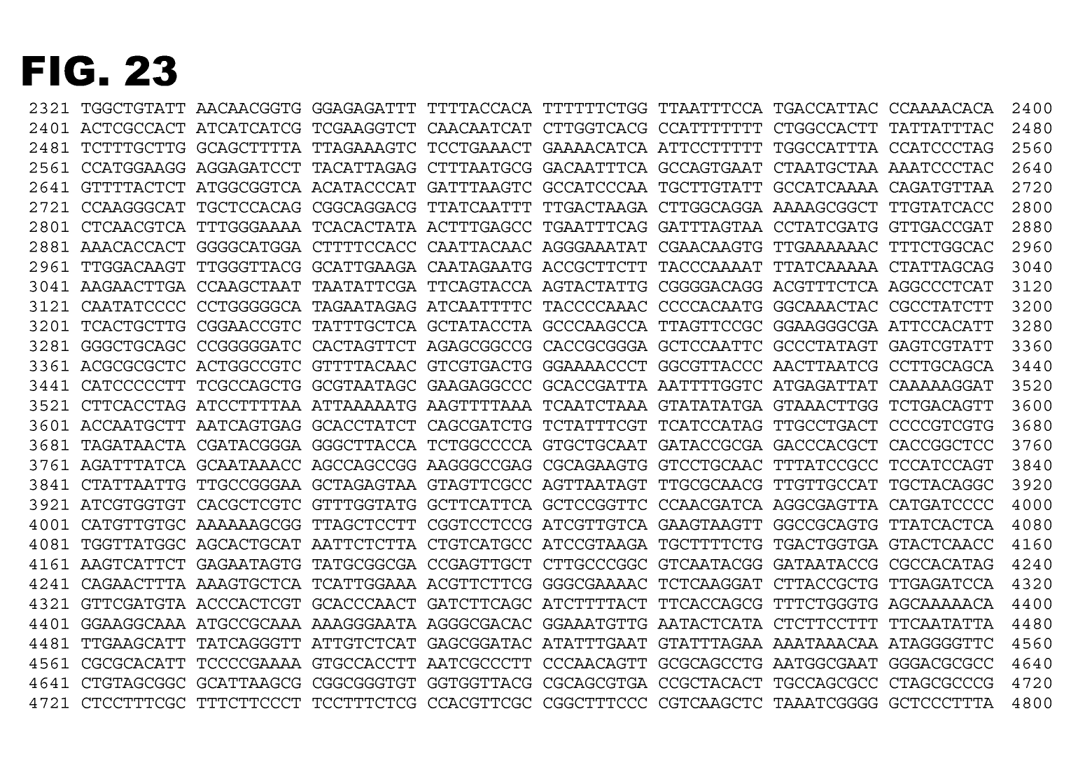

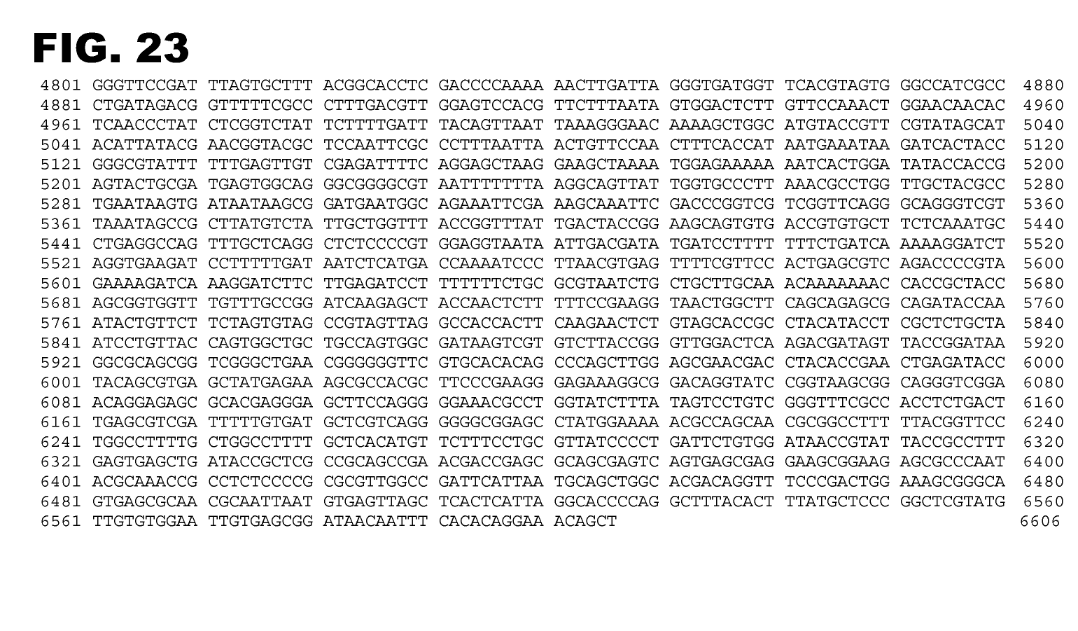

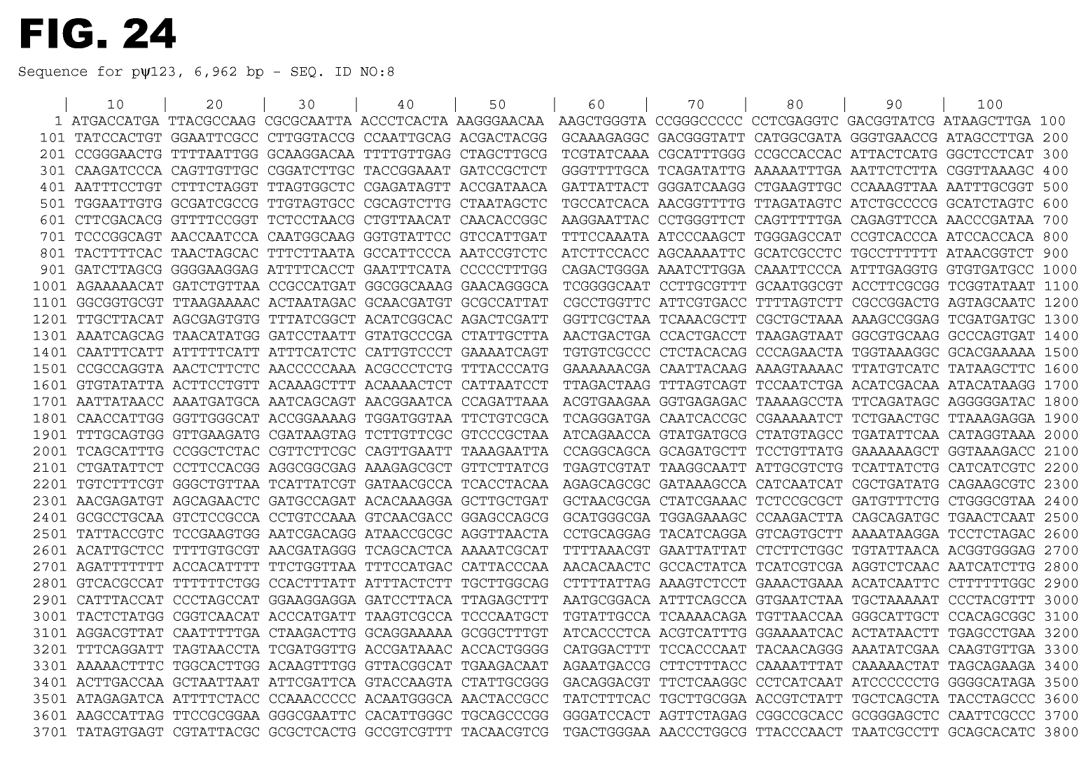

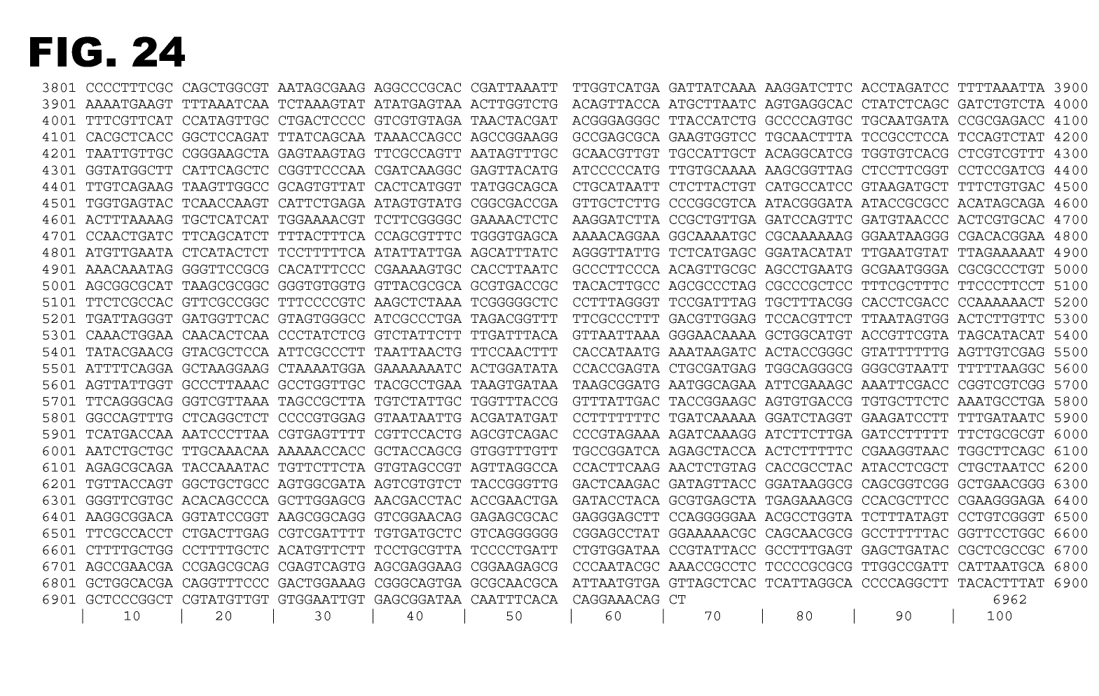

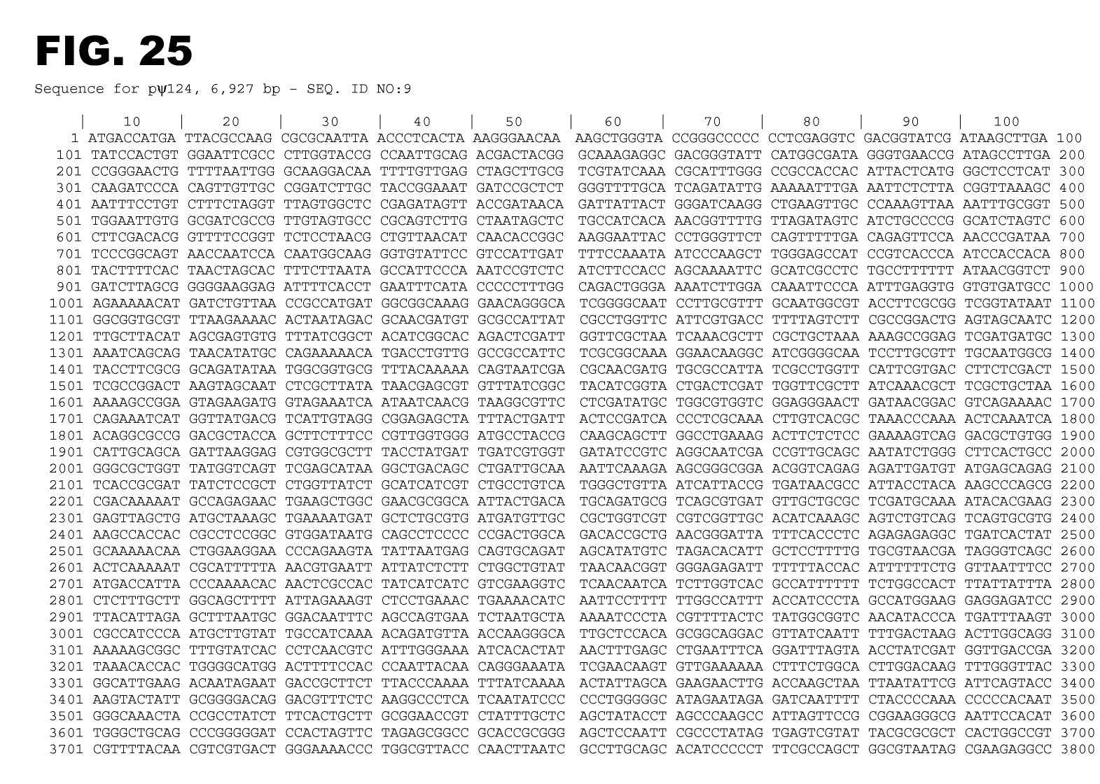

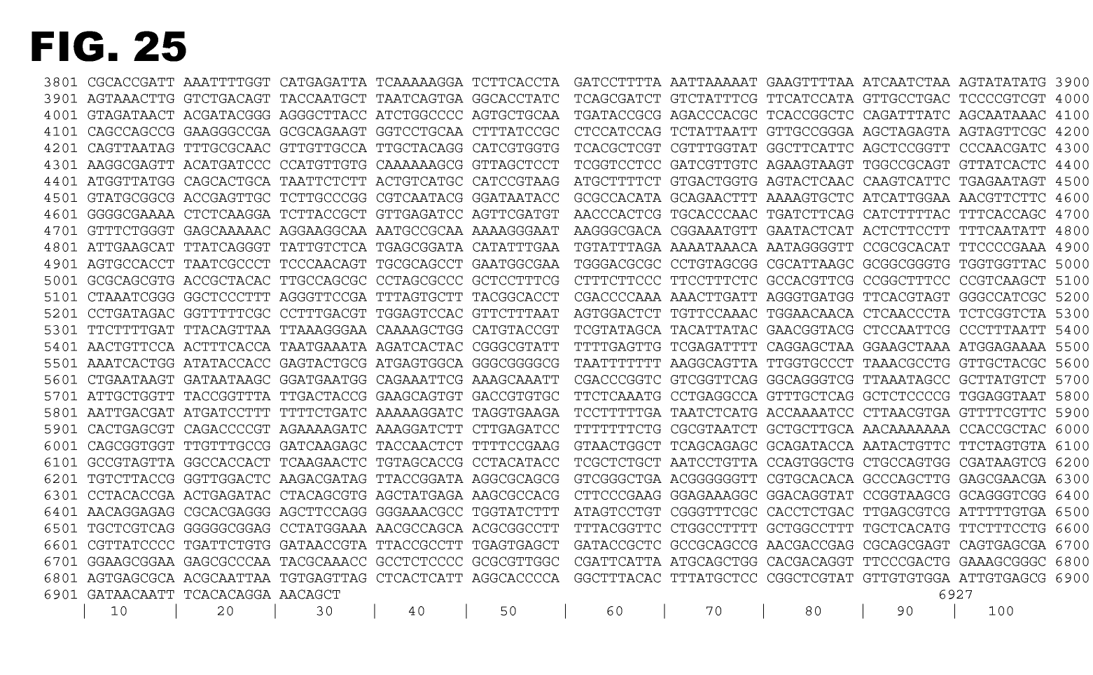

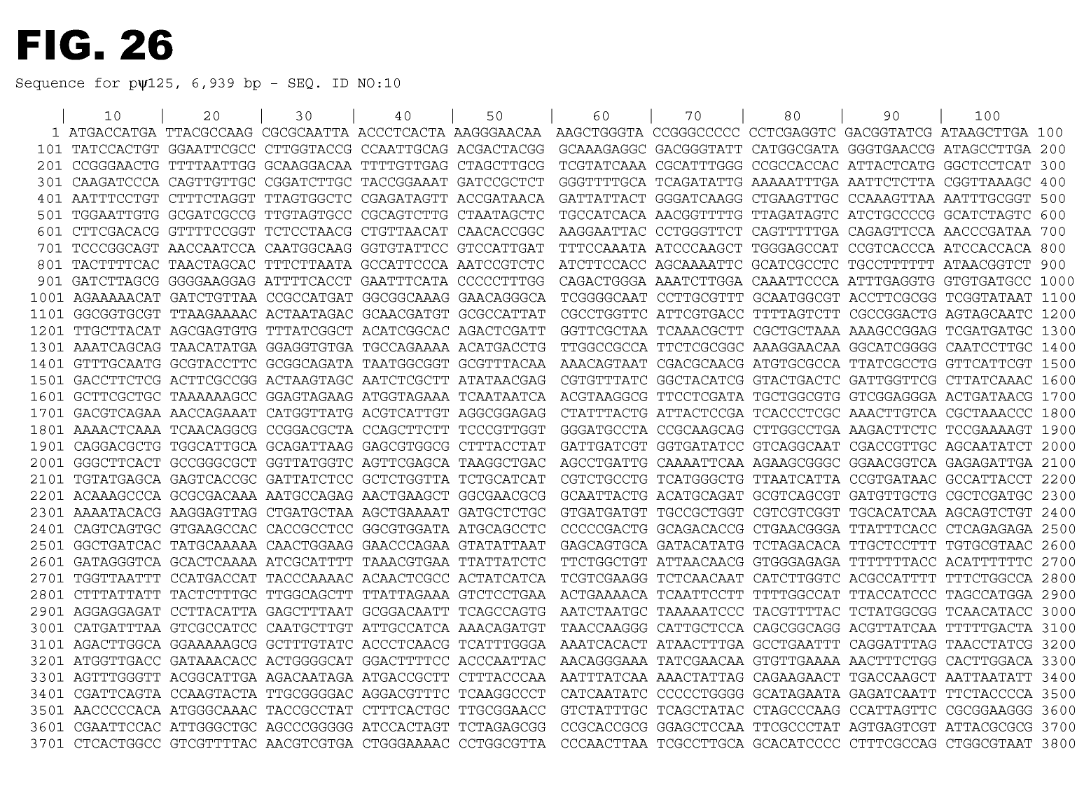

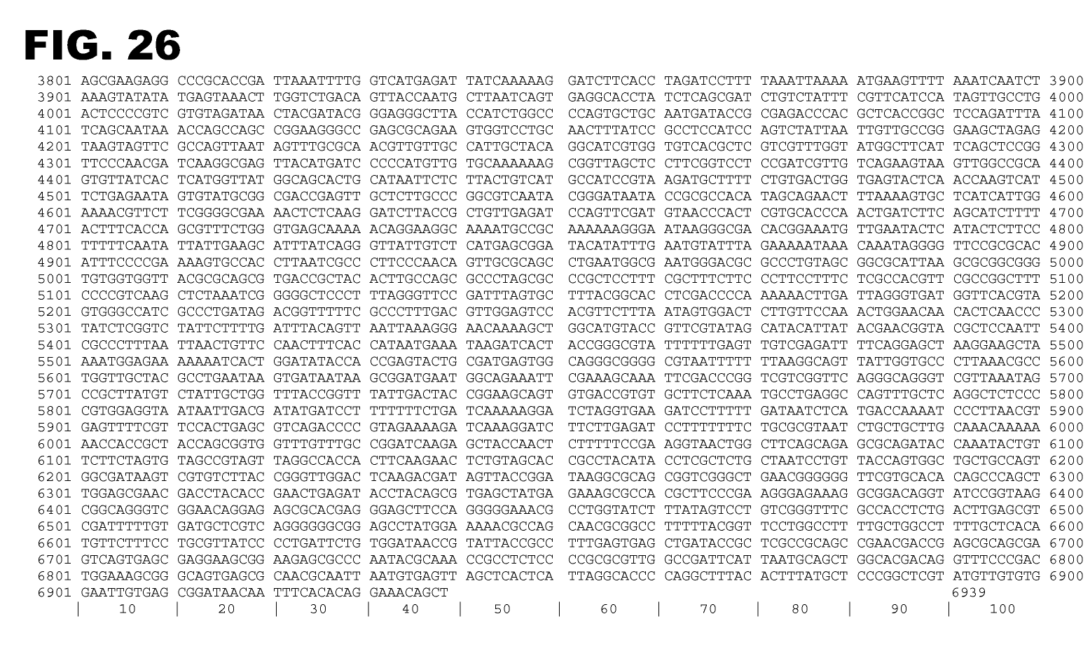

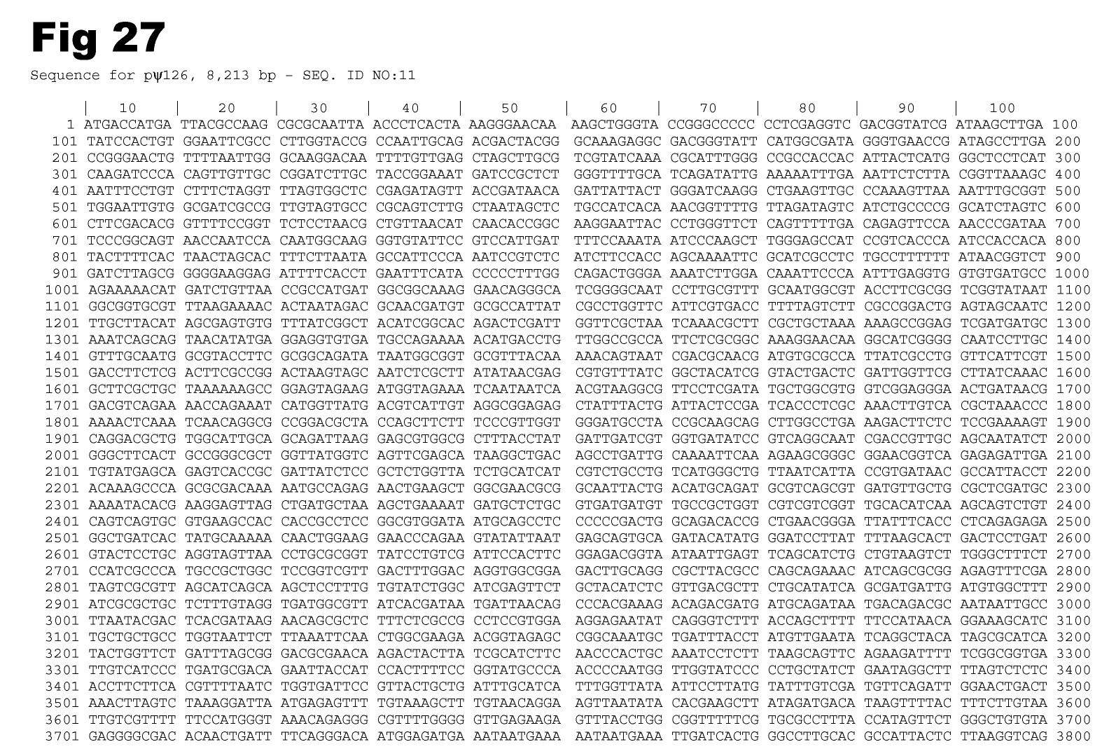

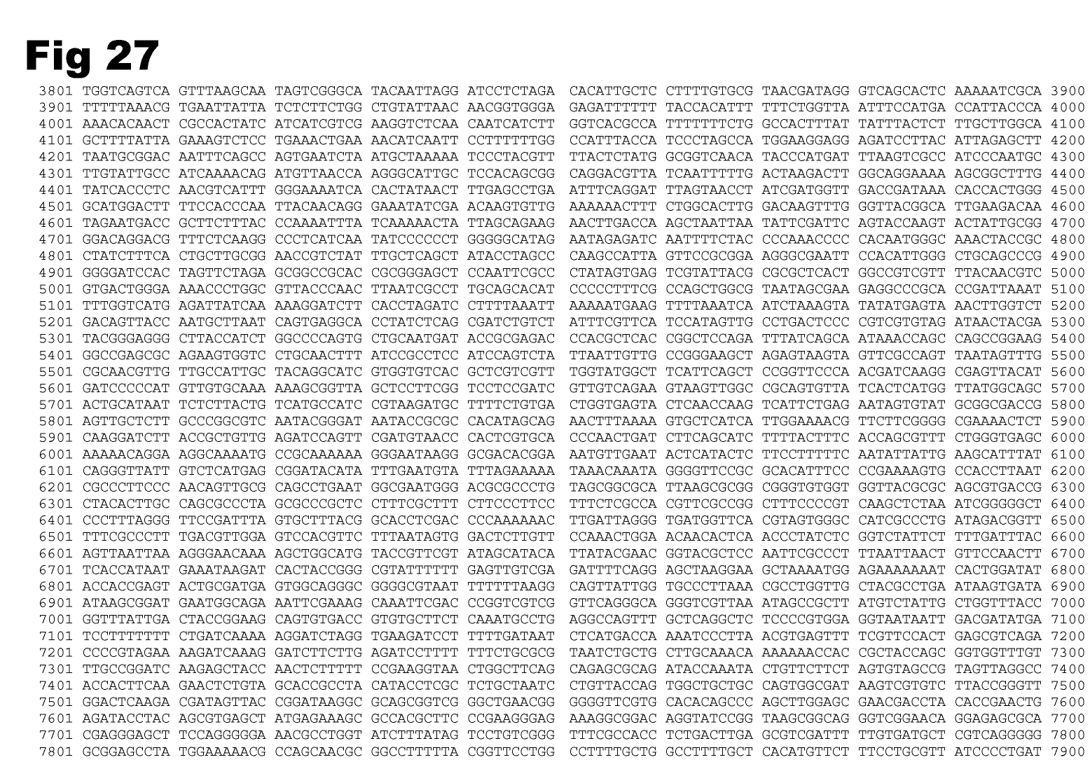

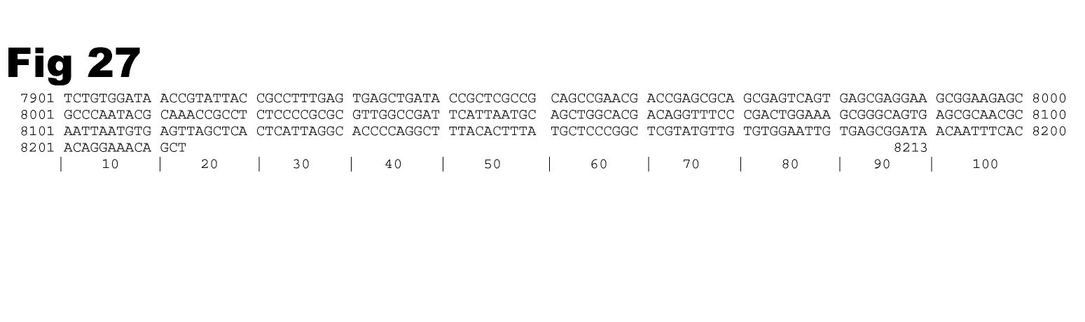

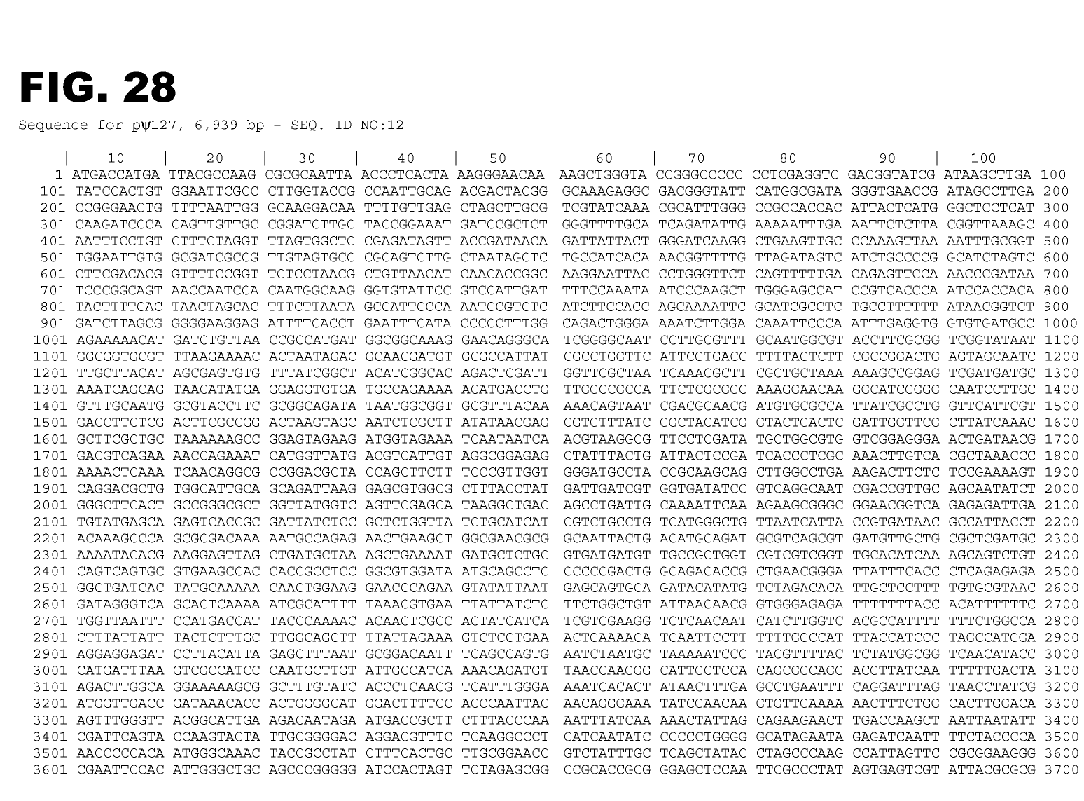

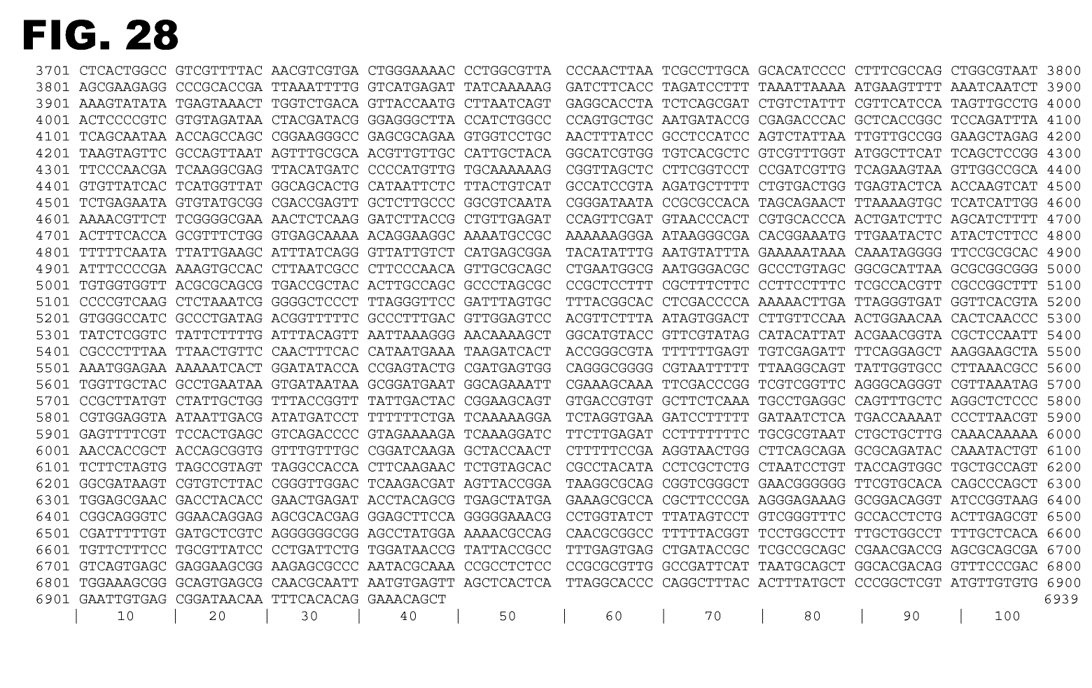

To ensure that the lysis genes from Salmonella and E. coli bacteriophages would work in Synechocystis, we made a temporary test strain SD101. Using overlapping PCR, three lysis genes from Salmonella phage P22 (genes 13, 19, 15) were amplified from a P22 lysate and fused downstream of a Ni²⁺ induction promoter (P_{nrsBACD}) to form a lysing cassette (FIG. 1) for generating pψ101 (Table 2, FIG. 19) that has the genes nsrBA deleted. The lysing cassette, accompanied by a kanamycin resistance marker, were set in the middle of two integration flanking DNA sequences possessing the inverted nsrRS genes (f1) and nsrCD genes (f2). This integration platform was transformed into Synechocystis by double crossover recombination (FIG. 1)

### Example 2

Example 2 gives the method for introducing the lysis genes into the Synechocystis genome without leaving residual drug markers. As shown in FIG. 3, a double selectable strain (SD102) is created, which cannot grow on BG-11 plates containing 4.5% sucrose (w/v) unless the Km^{R}-sacB cassette is replaced. After complete segregation of the double selectable strain, it was transformed with the markerless suicide vectors. The expected recombinants were then selected on BG-11 plates containing 4.5% sucrose.

Since rapidly growing cyanobacteria have multiple chromosomes and only one is involved in the initial recombination event, the level of resistance displayed will be initially lower than when after segregation has occurred and all chromosomes have the same genotype. After transformation, segregation without applying selection pressure is necessary for transformation efficiency. The phenotypic and segregation lags for sucrose survival (5 days) is longer than that for kanamycin resistance (1 day), because the phenotype of sucrose survival (recessive) occurs after all chromosomes have the sacB gene fully removed, while the phenotype of kanamycin resistance (dominant) occurs after enough chromosomes have the resistance gene expressed. Essentially, the selected colonies are genotypic mixtures of cells, so isolating test colonies derived from single cells obtained after growth of the segregating clone is necessary for obtaining a genetically pure recombinant strain.

### Example 3

Example 3 demonstrates three strategies to construct a series of markerless Synechocystis strains (Table 2) to achieve more effiecient inducible lysis response.

On the basis of the successful inducible lysis of SD101, three strategies (FIG. 4) are designed to optimize the system for faster lysis rates. Strategy 1 uses the lysozymes from P22 (in SD121) and λ (in SD122), respectively, to test the lysing abilities of lysozymes from different bacteriophages. It was observed that SD122 failed to lyse on Ni²⁺ containing plates, and its lysis rate in liquid culture after Ni²⁺ induction was significantly slower than that of SD121, suggesting that lysozymes from λ are less efficient than P22 lysozymes for Synechocystis lysis. These observations led us to utilize P22 lysozymes for further optimization.

Strategy 2 is designed to overexpress the endolysin genes (P22 19 15) under a strong Synechocystis constitutive promoter P_{psbAll} (Shibato, Agrawal et al. 2002), while restricting the control of the expression of the holin gene (P22 13). We presumed that before induced expression of the holin gene, the endolysins are accumulated in the cytosol. Once the holin gene is expressed, the holins synthesized would produce holes in the cytoplasmic membrane from within and allow the accumulated endolysins to gain access to the cell wall, resulting in destruction of the murein. The P_{psbAll} 19 15 cassette with a transcriptional terminator TP4 from cyanophage Pf-WMP4 (Liu, Shi et al. 2007) was inserted in different transcription orientation in SD123 and 124 (FIG. 4. Table. 1). The growth and lysis profiles of these two strains are not significantly different (data not shown).

Strategy 3 is to incorporate the lysis genes from λ with P22 lysis genes, with the assumption that different lysozymes attacking different bonds in the cell envelope will result in a faster lysis rate. As the constitutively expressing cassette P_{psbAll} R Rz is lethal for E. coli on cloning vectors, this cassette was transformed with an intermediate strain SD126 as an overlapping PCR fragment (Warrens, Jones et al. 1997) to result in SD127.

### Example 4

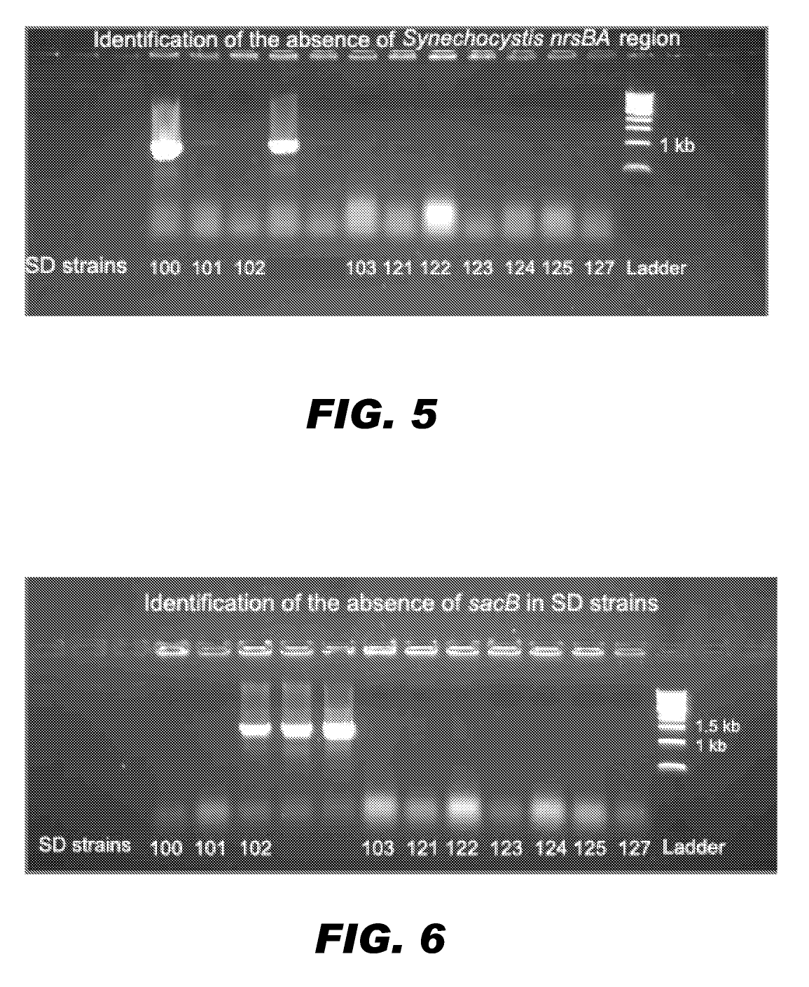

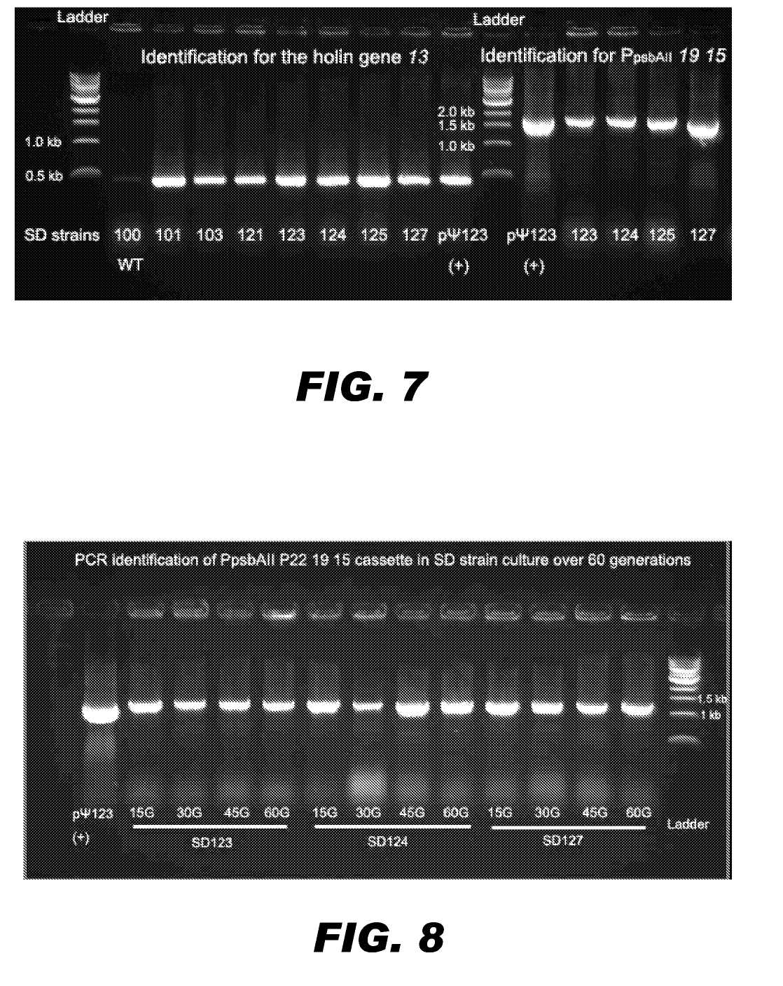

Example 4 shows the PCR identification of the lysis genes introduced into the SD strains. A long-term culture over a 75-generation period was performed to test whether strains segregated recombinant and non-recombinant clones and whether these lysis genes were stable in the host. The presence of insertions and absence of deletions were identified by PCR at a series of culture times (FIGS. 5-8). DNA sequencing data showed that all the sequences of the lytic insertions were correct as expected and also proved that the lysis genes were genetically stable in the Synechocystis genome over a period of 75 cell divisions.

### Example 5

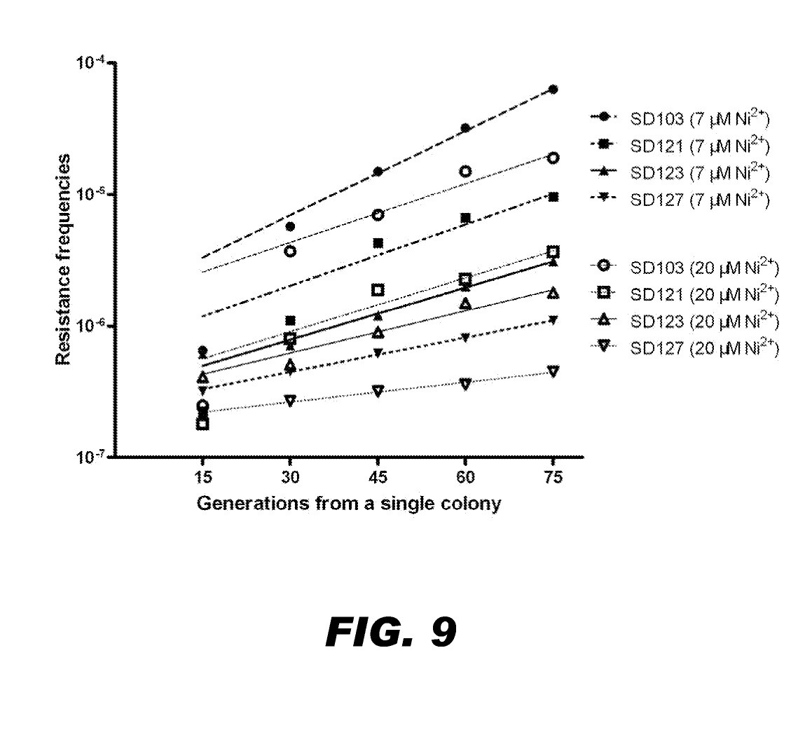

Example 5 provides the results of the Ni²⁺ resistance frequency test for the SD strains. Over a period of 75 cell divisions, Ni²⁺ resistance frequencies were evaluated by the survival ratio of the culture samples on Ni²⁺ containing BG-11 plates. This experiment was not applicable to SD122, because SD122 cells with the λ cassette can not be induced to lyse on Ni²⁺ containing BG-11 plates. As shown in FIG. 9, the resistance frequencies were low, at the level of 10⁻⁷. With the culture growing, the resistance frequencies caused by spontaneous mutation increased. According to the slopes of linear regression, the mutation rates to Ni²⁺ resistance for SD103, 121, 123 and 127 during the first 45 generations from a single colony were 48.2±5.7, 15.0±1.2, 3.1±0.02 and 1.3±0.01×10⁻⁹ per generation, respectively. However, the mutation rates determined by selection with 20 μM Ni²⁺ were more uniform with values of 17.8±2.4, 9.4±1.1, 2.5±0.05 and 0.8±0.01×10⁻⁹ per generation, respectively.

SD103 (with only one holin gene), SD121 (for Strategy 1), SD123 (for Strategy 2), and SD127 (for Strategy 3) cultures were grown from a single colony over 75 generations. The resistance frequencies were calculated as the ratio of the CFU/ml on Ni²⁺ containing BG-11 plates to the CFU/ml on the normal BG-11 plates. We predict that spontaneous mutations in the regulator genes nrsRS, in the promoter P_{nrsB}, in the binding site for the phosphorylated NrsR or in the coding region of the lysozyme genes could cause Ni²⁺ resistance. It was observed that the number of the resistant colonies on 20 μM Ni²⁺ BG-11 plates is fewer than that on 7 μM Ni²⁺ BG-11 plates (FIG. 9), suggesting that the resistant mutations are regressive, which means that the phenotype of Ni²⁺ resistance occurs after the resistant mutations are segregated and become present on all the chromosomes. It is possible that it will take the slower-growing stains (e.g., SD127) a longer time for the segregation of resistance mutations. On the other hand, a longer generation time might provide a better chance for the Synechocystis cells to repair the mutation, which would result in a lower mutation rate to Ni²⁺ resistance. In addition, strains with more lysozyme gene backups, such as the six lysozyme genes in SD127, will also result in a lower mutation rate to Ni²⁺ resistance.

### Example 6

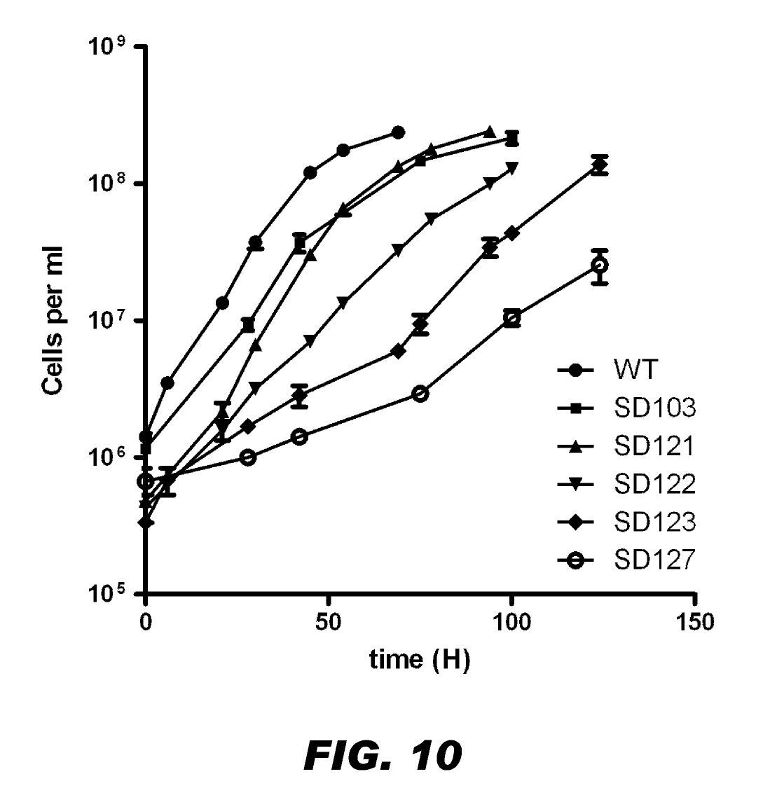

Example 6 shows the growth rates for recombinant strains. 300 ml liquid cultures were incubated in bubbling flasks with aeration of a continuous stream of filtrated air at optimal light and temperature conditions. The linear semi-log growth curves of the recombinant strains showed that the SD strains exhibited exponential growth at the cell density range of 10^{6˜10}⁸ cell/ml (FIG. 10).

Based on the data from the exponential growth period, Doubling Times (DT) for wild type, SD103, 121, 122, 123 and 127 were calculated as 8.13±0.71, 9.87±0.82, 11.07±1.18, 15.10±1.43, 14.13±0.84 and 17.68±0.72 hours respectively. The growth rates for SD103 and SD121 (Strategy 1) are not significantly different from wild type, while the growth of SD123 (Strategy 2) with constitutively expressed endolysins was significantly slower than that of wild type. The growth rate of SD127 (Strategy 3) with combination of lysis genes is the lowest of all the constructions. We observed the unhealthy growth of SD123 and 127 in the air-bubbled flasks, where the growing cells aggregated into clumps and attached to the vessel walls. These phenomena suggested cell walls were compromised before induction, which may be caused by leakage of the internal endolysins. We speculate that a cascade induction strategy would be able to lyse the cells without slowing the growth rate. Instead of constitutively expressing the endolysins, we can use another inducible promoter to induce expression of the endolysin genes a certain time before the induction of the genes for holins, so the endolysins would not accumulate in the cytosol during biomass growth.

### Example 7

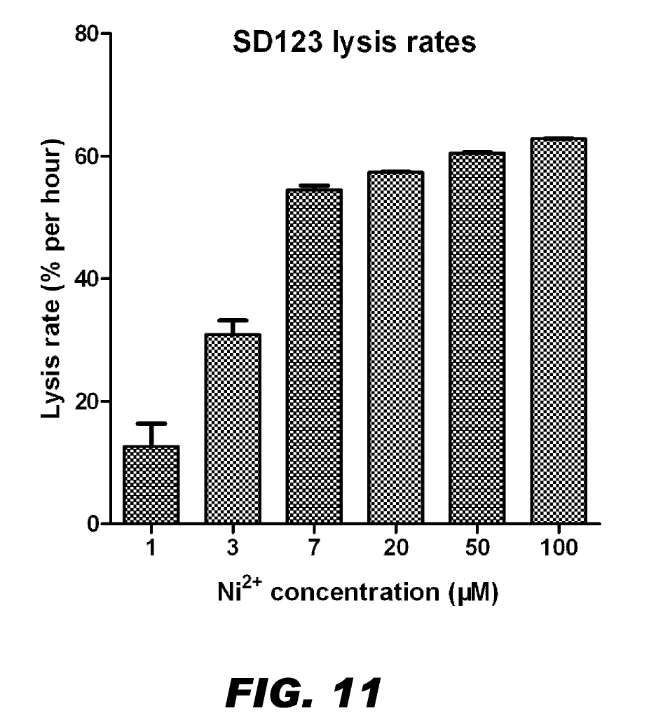

Example 7 shows the lysis responses of recombinant strains in liquid culture. The initial culture concentrations were adjusted to 0.5×10⁷ cells/ml (OD_{73O nm} ˜0.3). After addition of NiSO₄ to the cultures, a lysis response was induced in the recombinant cells, which was usually accompanied by foaming. Lysis responses were measured by determining the decrease of viable cell titers as colony formation units per ml (CFU/ml). Based on the slopes of the decline in CFU/ml, the lysis rate increased with the Ni²⁺ concentrations from 1 to 100 μM (FIG. 11).

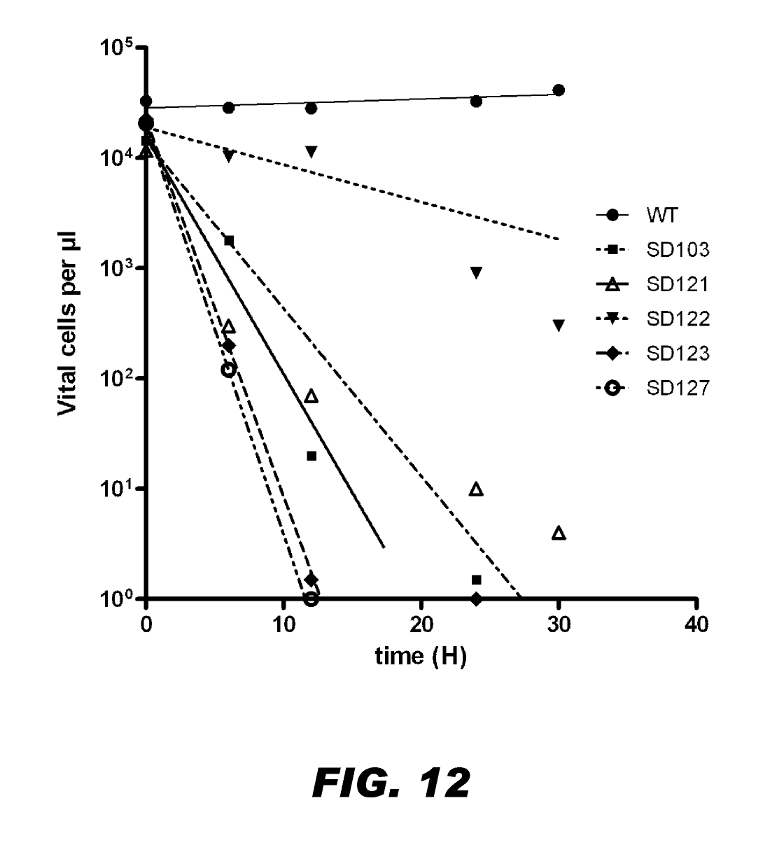

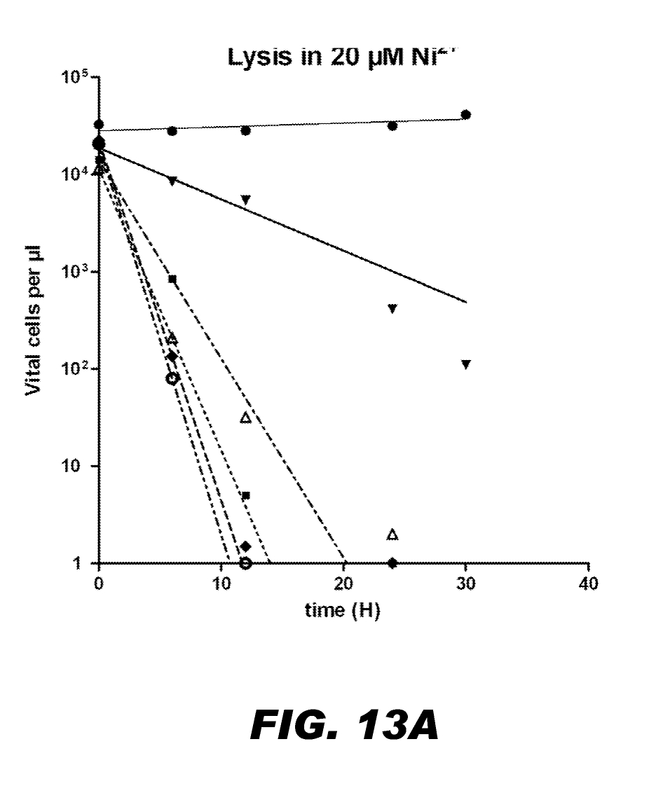

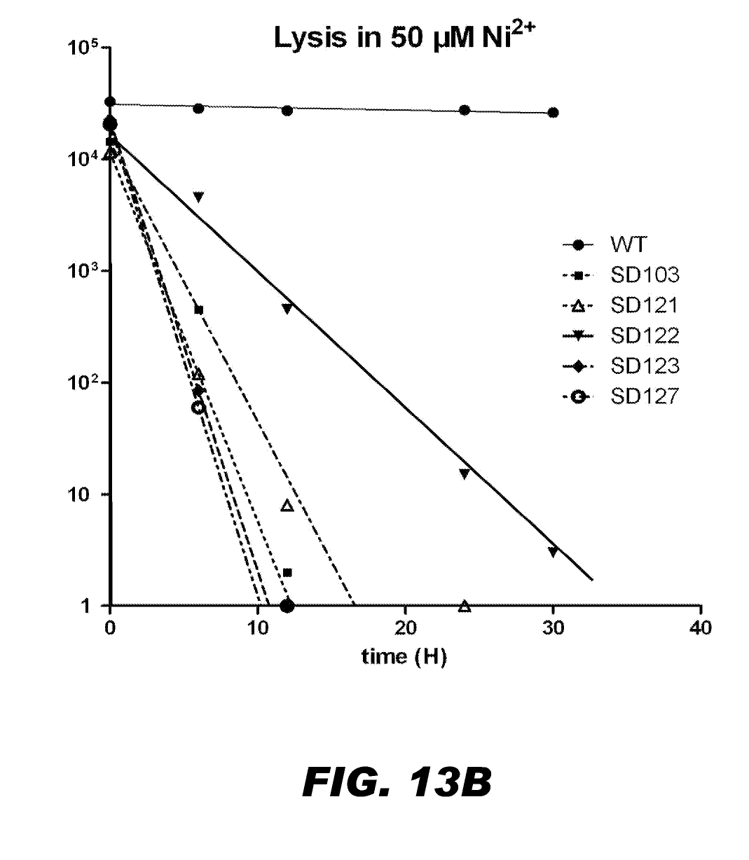

The lysis responses of SD strains in liquid culture with addition of 7.0, 20 and 50 μM Ni²⁺ (FIG. 12 and FIG. 13) shows that at the higher Ni²⁺ concentration the lysis rates of different strains became closer to each other and to a saturated level of about 60% per hour (Table 3). The data indicate that SD121 with P22 lysozymes lysed more rapidly than SD122 with λ lysozymes, and the lysis by Strategies 2 and 3 (SD123 and127) was faster than that by Strategy 1 (SD121).

TABLE 3
Comparison of different lysis strategies

| | | Doubling | Mutation Rate ^{a} | | | | |
|---|---|---|---|---|---|---|---|
| Strain | Lysis Strategies & | Time | (10⁻⁹/generation) | | Lysis Rate ^{a} (%/hour) | | |
| SD No. | Descriptions | ^{a} (hour) | 7 μM Ni²⁺ | 20 μM Ni²⁺ | 7 μM Ni²⁺ | 20 μM Ni²⁺ | 50 μM Ni²⁺ |
| SD100 | Wild type Synechocystis | 8.13 ± 0.71 | — | — | — | — | — |
| SD103 | Only control phage P22 | 9.87 ± 0.82 | 48.2 ± 5.7 | 17.8 ± 2.4 | 29.52 ± 2.42 | 37.59 ± 1.02 | 43.83 ± 0.46 |
| | holin gene 13 | | | | | | |
| SD121 | Strategy 1, using P22 | 11.07 ± 1.18 | 15.0 ± 1.2 | 9.4 ± 1.1 | 45.39 ± 1.84 | 48.71 ± 2.10 | 53.31 ± 0.81 |
| | lysis cassette (13 19 15) | | | | | | |
| SD122 | Strategy 1, using phage | 15.10 ± 1.43 | — | — | 7.5 ± 3.23 | 11.46 ± 3.17 | 14.10 ± 2.76 |
| | λ lysis cassette (S R | | | | | | |
| | Rz) | | | | | | |
| SD123 | Strategy 2, control P22 | 14.13 ± 0.84 | 3.1 ± 0.02 | 2.5 ± 0.05 | 54.49 ± 0.73 | 57.37 ± 0.11 | 60.54 ± 0.10 |
| | holin gene (13), while | | | | | | |
| | constitutively express | | | | | | |
| | endolysin genes (19 15) | | | | | | |
| SD127 | Strategy 3, combination | 17.86 ± 0.72 | 1.3 ± 0.01 | 0.8 ± 0.01 | 57.54 ± 0.03 | 60.32 ± 0.10 | 62.18 ± 0.16 |
| | of P22 and λ lysis genes | | | | | | |

^{a} The growth and experimental conditions for Doubling Time, Mutation Rate, and Lysis Rate are defined in the Materials and Methods section

### Example 8

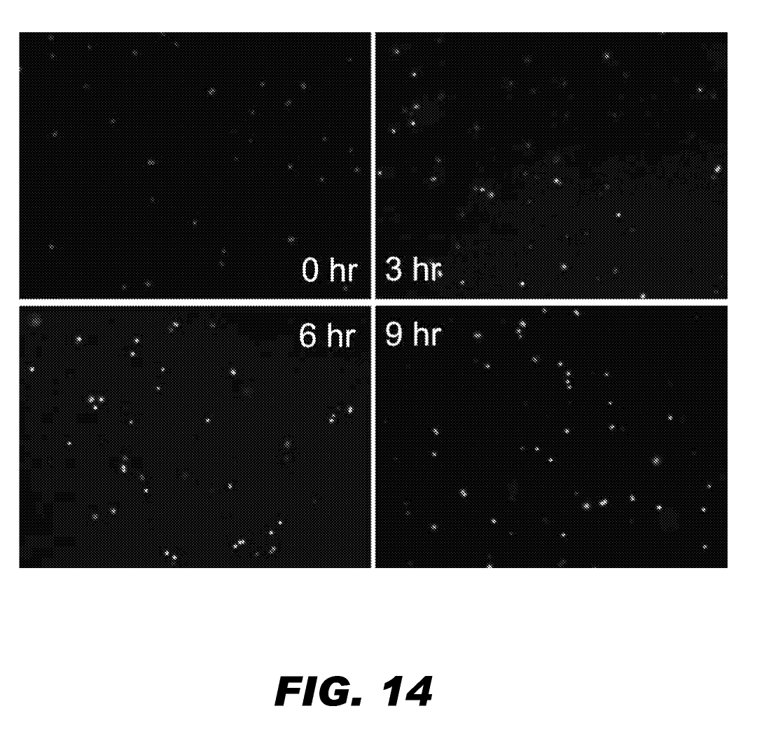

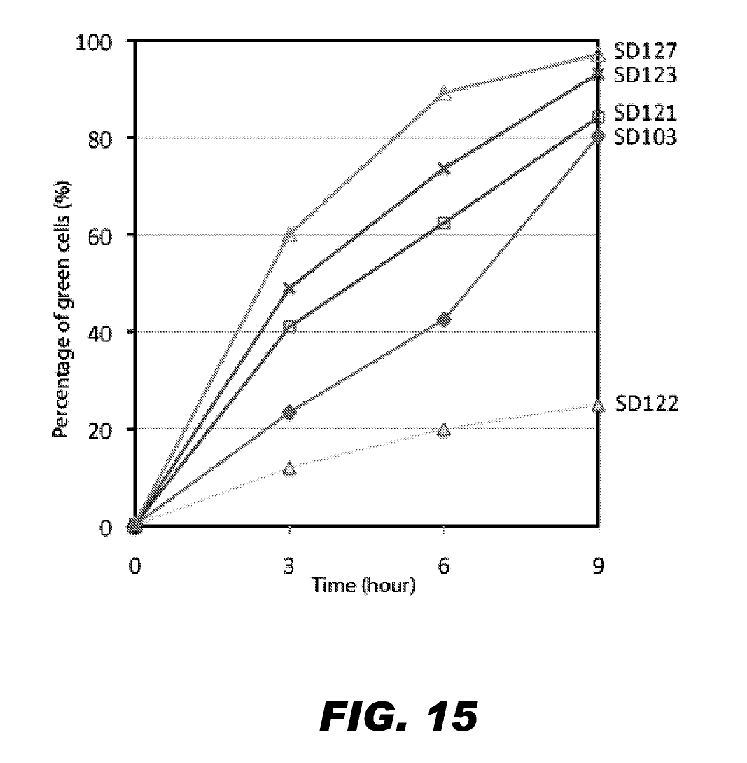

Example 8 shows the penetration of dye through the lysing cell envelope after Nickel addition to the culture The leaks created by holin-lysozymes on the cell envelope were indicated by penetration of SYTOX Green nucleic acid stain (Invitrogen Molecular Probes, Inc. OR, USA). The stain easily penetrates the compromised cell envelopes and yet will not cross the membranes of live cells (Roth, Poot et al. 1997). After brief incubation with SYTOX Green stain, the nucleic acids of lysing cells fluoresce bright green when excited with 450-490 nm spectral sources, while the intact cells emit red fluorescence of phycobilin (FIG. 14). The penetrable cell ratio in lysing cultures increased with time after Ni²⁺ addition (FIG. 15).

### Example 9

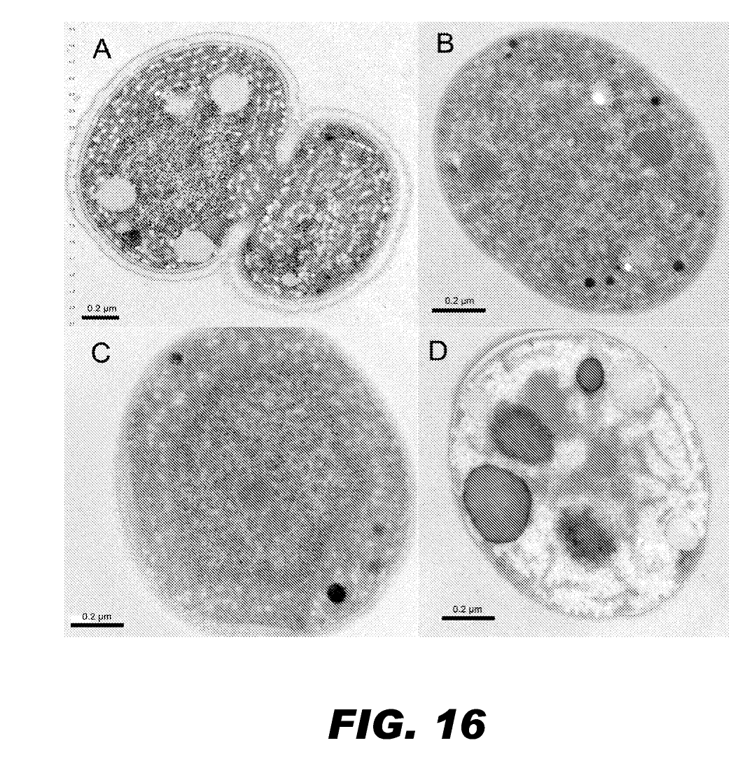

Example 9 displays the transmission electronmicroscopy (TEM) images of SD121 that show that the expression of lysis genes cause the cell wall (peptidoglycan layers) to decrease in thickness 6 and 12 hours after 7.0 μM Ni²⁺ induction and the cell structures to degrade 24 hours after Ni²⁺ induction (FIG. 16).

### REFERENCES

Bertani, G. (1951). “Studies on lysogenesis. I. The mode of phage liberation by lysogenic Escherichia coli.” J Bacteriol 62(3): 293-300.
Black, T. A., Y. Cai, et al. (1993). “Spatial expression and autoregulation of hetR, a gene involved in the control of heterocyst development in Anabaena.” Mol Microbiol 9(1): 77-84.
Kufryk, G. I., M. Sachet, et al. (2002). “Transformation of the cyanobacterium Synechocystis sp. PCC 6803 as a tool for genetic mapping: optimization of efficiency.” FEMS Microbiol Lett 206(2): 215-9.
Liu, X., M. Shi, et al. (2007). “Cyanophage Pf-WMP4, a T7-like phage infecting the freshwater cyanobacterium Phormidium foveolarum: complete genome sequence and DNA translocation.” Virology 366(1): 28-39.
Loessner, M. J. (2005). “Bacteriophage endolysins—current state of research and applications.” Curr Opin Microbiol 8(4): 480-7.
Oka, A., H. Sugisaki, et al. (1981). “Nucleotide sequence of the kanamycin resistance transposon Tn903.” J Mol Biol 147(2): 217-26.
Rippka, R., J. Derulles, et al. (1979). “Generic assignments, strain histories and properties of pure cultures cyanobacteria.” J Gen Microbiol 111: 1-61.
Roth, B. L., M. Poot, et al. (1997). “Bacterial viability and antibiotic susceptibility testing with SYTOX green nucleic acid stain.” Appl Environ Microbiol 63(6): 2421-31.
Sambrook, J., E. F. Fritsch, et al. “Molecular Cloning: A Laboratory Manual. 2nd ed. (Cold Spring Harbor Laboratory Press, Cold Spring Harbor, N.Y., 1989).”
Shibato, J., G. K. Agrawal, et al. (2002). “The 5′-upstream cis-acting sequences of a cyanobacterial psbA gene: analysis of their roles in basal, light-dependent and circadian transcription.” Mol Genet Genomics 267(5): 684-94.
Warrens, A. N., M. D. Jones, et al. (1997). “Splicing by overlap extension by PCR using asymmetric amplification: an improved technique for the generation of hybrid proteins of immunological interest.” Gene 186(1): 29-35.
Young, R. (1992). “Bacteriophage lysis: mechanism and regulation.” Microbiol Rev 56(3): 430-81.
Young, R. (2002). “Bacteriophage holins: deadly diversity.” J Mol Microbiol Biotechnol 4(1): 21-36.

1. A method for degrading the peptidoglycan layer of the cell wall of a gram-negative bacterium, the method comprising:
a. introducing into the bacterium a nucleic acid comprising an inducible promoter operably-linked to a nucleic acid, the nucleic acid encoding a first protein capable of forming a lesion in the cytoplasmic membrane of the bacterium and at least one endolysin protein; and
b. inducing the promoter to express both the first protein and the endolysin, wherein the first protein allows the endolysin to degrade the peptidoglycan layer of the cell wall.

2. The method of claim 1, wherein the gram-negative bacterium is a cyanobacterium.

3. The method of claim 1, wherein the inducible promoter is induced by a metal or metal ion.

4.-5. (canceled)

6. The method of claim 1, wherein the first protein is a holin.

7.-10. (canceled)

11. The method of claim 1, wherein the nucleic acid comprises at least two endolysin proteins.

12. The method of claim 1, wherein the endolysin is selected from the group consisting of a lysozyme, a transglycosylase, an amidase, and an endopeptidase.

13.-34. (canceled)

35. A gram-negative bacterium comprising an inducible promoter operably-linked to a nucleic acid encoding a first protein capable of forming a lesion in the cytoplasmic membrane of the bacterium and at least one endolysin protein.

36. The bacterium of claim 35, wherein the inducible promoter is induced by a metal or metal ion.

37.-38. (canceled)

39. The bacterium of claim 35, wherein the first protein is a holin.

40.-43. (canceled)

44. The bacterium of claim 35, wherein the nucleic acid comprises at least two endolysin proteins.

45. The bacterium of claim 35, wherein the endolysin is selected from the group consisting of a lysozyme, a transglycosylase, an amidase, and an endopeptidase.

46.-49. (canceled)

50. A gram-negative bacterium comprising:
a. a first nucleic acid, wherein the first nucleic acid comprises a first inducible promoter operably-linked to a nucleic acid encoding a first protein capable of forming a lesion in the cytoplasmic membrane of the bacterium; and
b. a second nucleic acid, wherein the second nucleic acid comprises a second promoter operably-linked to a nucleic acid encoding at least one endolysin protein.

51. (canceled)

52. The bacterium of claim 50, wherein the second nucleic acid does not substantially affect cell growth prior to inducing the first promoter.

53. The bacterium of claim 50, wherein the first inducible promoter is induced by a metal or metal ion.

54.-55. (canceled)

56. The bacterium of claim 50, wherein the second promoter is a constitutive promoter.

57. The bacterium of claim 50, wherein the second promoter is an inducible promoter.

58. The bacterium of claim 57, wherein the first inducible promoter is not induced by the same condition as the second promoter.

59. The bacterium of claim 50, wherein the first protein is a holin.

60.-63. (canceled)

64. The bacterium of claim 50, wherein the second nucleic acid comprises at least two endolysin proteins.

65. The bacterium of claim 50, wherein the endolysin is selected from the group consisting of a lysozyme, a transglycosylase, an amidase, and an endopeptidase.

66.-87. (canceled)
